SEQUENCE LISTING (24 sequences)

SEQ ID NO: 1 - DNA, 2000 bp, Manihot esculenta
  source 1..2000: mol_type = other DNA
ataagacggtatacataacttataaaactaaaatatttataattaggatatatcaatcacatataaataattacgaactaattaaatatttttttttcacgtatcgtacgtagaaagtctcacctcaatatgtcactatgtgacatgacatatttttgctgattatataatttgccttaatactttgaattggggcatataagagagtgatttagtttcgtattacataatgactttgattcaaaaattcacaaattattgataatatgaatatgaaaatcttatatagattaaattaataatatgttataggggatgatatttactaaatcaaaacttttaaaatattgttgaaattttgattcgaatgtaattaataattcaaaataaaataaatagtatcttattattagagttttatttacataaatttattattttatttttaaatattactattaataggttatttcatatatttttaaaaatttattaaaatatttttatttttttttattaacaaataatttttttatttaatttcgtttatttttgtcgttaaatatataataaaaaattaaatttttttaacttttaaaataaaattttaaatatataataaaaaattaaaattttttttatcgattaaaaagtacttttaaaaattaagttaatgaagaagaagaaagagaaattgagttatccaaaaagaagagaaaaatttgagagtcgcatgcattaaagtcacagcgtaagtaattaattaatataaatgactatttgagagaaaaaaaaaatagtggttaaaattatttttttattaataaattattataaaataaatttacataaattatattaaattttatatttttttattatttataagtatttatcgtaataaatatttataacaaaaactttgaatacgaatcattcatatataaagttggcattgaattaccatatttgtaaaggaggtaaaaatggtgaggcagttctaagttggatctcaattattgcgtgatagtttttttttctaattcaacagcacatgagagaggctcatctctgctacctttaacttccttatctcatagtttctaattaggacttttcttccctctttttccatttatagtaatttctgattaacattaatttctatttatcatatttcagaaatttgttttaaaaaatttataaccttgaaaagtttcctttcaaaaagtagatctgaagtgaagtaactttttttataaattaatcaatttttttattaatttactgtttactgaaattcaaataggagactttataatcttagggtaagaaatttttaactttattaactataaatgtaaaatttgaaatatgtcattcctttcacaaatattacaattctcacaattatttttaaaaataaatatttaattaataataacatctaaatcaagaatatcaggaaagtttttttttttttttttttttaattgtggtggagttggacttaggtatactagatcttttaattcatctcaacttttgaacaacctcagagaaatttacaaataatttcaaaatattataaattagaatatgaatttataatataaatatatagaaattttatataaattttattattattttacagtgtaaaatcacgaatctggctattattttacgctataaatctacatagaccgcctaatctttttcctcaaggtgcagcgcccattccctgtgagttactgttagcccactgttcatagacatcgctcgaattaccaaagcaacttgaaaacaaggaaatttaaacctcgctgttatttgtttccttctctatataatccattgccacttccctcttccactcatcgcattgacatttgtgaaccaacctctgaactttcagtttgattcttcttgaaactatttgtttcttgtctttgccttctcttttcttttgaaggttatttctttcaatcttgctgaaa

SEQ ID NO: 2 - DNA, 2000 bp, Manihot esculenta
  source 1..2000: mol_type = other DNA
tgcaattatttaagttggtttctctataataatgaatttaaagattttctaactaaatatttatttttctctcccttttgaaatattttaattcatattattttattattaaattattttttaataaaatatattatttgatatatagcaattaattttaaattttacggcaaatttaaatcgtatataatttttactatccacattcgatagagattcacgatagttgttcagaaagaaaatttcgagatatataatttttaaaagtgtgacgagaaccatcagcctaatcacaaaattcgatacaaaaattttatcgtttagaaatttaattttatattttaattattttttaaatattttaaatattttcatattttcatatattaaaattttattttctattataaatagcatctcttatttattaaaaactcactttgcagaaaaattttaataagagttttaattttttatcttattttttctcttatttcatcttaacataacaatattgattaaaattctgacggaatttaaatagttatttatcattattatttaatttttattttataaaaaaactgactaattaaattatttgatattttaaaaaatatactatttaattttatttattaaatattataaaaaattttaaaatattttaatatgaaagaattaattgataaatatcatttcaaaaatatatatattaatttatgattgatagaaaaaaaataattaagcatgtatttttgaaatttatacatatattattattattatttaaatcatttatatattttaattaacaatataagaatcatatagcaaatattctatttaaaaattttaaattattaagattttaattaaattggtttttctttaaaaatggtatcaaatcaatattctctttgtatcgaattggaatcaaatcgatttgaatttgagaaaaattatttttttttcttttagaaaaattaatcattatattgaatgaaaataaacatttgaaacaaaacactcaaaacagtccattttggggccacatttctttaaaaatttttaagagttaaataagttttgatttactaataaaatattatttttataaattaaaatattaaaaaatttagtattttaatcaatataaaatttttaaaaaatacacagaaatagtaagaaaaataaaattttattattaaaaaattatattaaattagaatttatttaaccttgcgcaaatgaggtataattgaaaaagaaaaattaaacttatttaattgttagcaaaaattacaaaagcttatttggaatttatttgcaggaaaaatggacggctggaaatacatgagaaaggggatggaaatatcctcccattcaacaattacatcaatcaatttgtgaaagttggccatgctgactaaataaaatactattcagaaatttaaaccgtttgaaactttcaaattcttcttgcacggctgtgtttagagattcagacattttaaaaatgtggaaatattttatgaagctcggttcaatgaacagaattttggaaaaaaaaaaaatttgcctttgatttctcataaaaatccaaataaatgaaatattattacttaaaataatattttgttaaattcatttcttattgacttattgtgatgcaggcaatttctaaattgtatcattgcttcacagactgagagtcatggcctcttcccatataaacaccctctcctccaacctctctccagtagcatacagcttcctttcatctccaatcccgacaacccctctcctgtcctcacttcttcacaattctggttttttctcctatatatatttcccctacattctcatattttcactggaaacaaacagagtttcaagaaattctctttcaagtattctttatctgtgtgcttcttttgataccagtaacgttaacacattcttttgtttgtagctgtaagcctgtactctactagcac

SEQ ID NO: 3 - DNA, 2079 bp, Manihot esculenta
  source 1..2079: mol_type = other DNA
catgtgaatggtgggtaaaataggcaaagtaaaaaaaccaaaaaaaagtcaactagcaaaggcgtcgtcatgactgattcagctgcgttttttttttttttttttttgggaatttgtagacttaggaggaaggaattcagcaaggacgaggaagcgagaatctatctatgctcgttccttccttttattttttttaattaatttttttaggcacaattctcacatgttgtaaatataatatttttaaattacttaattactttcttataatttttaattaccggaaagatcaaaataattcaacctcaattttcaatgtttaatatgttatactcaaagtttgacggctgaatatattataaaaattgatgtgcgaggagatttttaagggtttttaaattttttcatattatatacgaatttttttttattaaagcaaatgatatttatttataaatatacagaggcgccatgataacagtgagataagttagatttagcaagatattcaaataagaatatttagataaagatttaatttatcggattaattatcgttctgtaataagttcaatatttgactttaaaaaataaaatctatttaatatttgactttaaaaaataaaatttattaaataaaaaagacggttaagagatcactaaaaaattttatgtgaaaactctccgatactcaaattagaattgaaaacttcacgaaattaaggaaagtaaagcagagagagaaaataagagaatggtggttttgaattcttcatgagtgaccgttcgagagaaatgtgtcttttttcagagaatgagagatctcttttttagaattagtttgatacataaatactttttaataaaattgatgtccctagtgtcacatttccttctaatagaatagagatatttttgtgtagatcaggtattacattaatgatggagtataggataataaatgtgaaactgatcggttcataccttttatattaaatgggcgtttatgattttttggactaaattaaagaggcgataaatttatagaagattgttatttttaccctgacttcatactgactcatccgacctatatagaaataaacctttttcagttaatgaaatcagccacatcagcgttttaaactttgttgtagtaaagatttttaaggaggtcgatctgtttgtcatttctagtctttgagaaggtaaatctatatgttccggacttaaataaatttaagagtgttgctcgtatgatttttatactcaatagattgaatttcttatcgaaaagattttcaattcacatatacaaatataatatatatatatagtattttattttttatacattatcatgctgtaattaaattaaaaaaatatatattgtcacaatttgaattttaacacttgtttaaaattgtgccatctttaatttgaatttattttcgatcaaatcaatcgatatattcacatattcatctttaatttaaatttagttttatcattatgagattcaaattatattataattaaaaaaataaagaaaatatctaataaaccttattattttgattagtttttggatttattagttttattatataaaaaagtgattaatagattcaattaatttgtaataatttattgtatagtataaaattgataaattaattgaataaagcagaatggaatacgtaaatactgtcttatgctttattaaacataaattttataattttttttgagagaataaattttataattaatgatgtagaagattattgcaaatgtttaaatgtttaccactaaataaaataaattttttttataaataattagaaccaatggagtgactccacgcgtgcaaggcatggatgaaaggtgaaggatttgcaaccacccatcctgataacgtttgcaacatcatatccagatataaaaccacaaaacctggacacgtggtgaagtgagaatcactattcagtaatttaattattttatttatttatttatcagtgcaacattttacttgaaaagattgaaaggcaaagaagagagaagagaagaaaacaa

SEQ ID NO: 4 - DNA, 1999 bp, Manihot esculenta
  source 1..1999: mol_type = other DNA
gcaagttgtaatgacagagaggcaattgccctacaccagttagccaagctccatgctgagcttggacgttctgaagaggcagcactttactacaagaaggacttggagaggatggaagctgaagagagagaaggaccaaatatggttgaagctttgctgtttcttgcccaacactgcagagagcagaagagatttgaagaagcagaggtgtattgtacccgccttctcgattatactggtccagtgagtttctatgatctaatatctatgaattgaattaaaaaattgagatccattccataagttccaatttataaatgtaaattaggttcattctctttggtttatctgtttatggtttcaggagaaggaaacagcgaagagtatgcttcgaggaatgagaacatcagaatccagttttcctttaatggatattgagcattttcatccctgaattcaaattctatatagtatgtctcccttgtctcatttcatgctcatcaaaatcagctatgatgaagtttttgtaatttgtcaaattgtgatacctaaatatgtgtctgtagtttaatgtttcataggtgttgacttcttctgttgttactctccttttaagaaaaaagaatgctgttatgagagcagtaaatttgtctgaatgtggcatggtaatttctagaatacatggctatcattcatgagccaatgtcaatgttgtactcttaactttgcaatgtcaatgtcgccgccctcttgtaatgctgctgaagcttcatgggcaatggtcagcaagatatccataccaaaagagagaacttccctgttgtattagttttgagtgaaatgaagcattcacgtttctcaaatgatatgcaagagtcaagactttgattaatcttattttcttccccggaggggcaagtattgggtcttgaagtgcaatgggagtggcaacccagtcctttcgatgacaagaactagatagatctctgcaacactcttagaaatgtgttgtgcccagagtcaaggaagtggttagtgcgggtaattcagaagaacagtaaactaatagtagtctaacagctaagagagatctcattgcaattgtgattttttctagcttgagttcttttgcaaaatcttcaggccttaaaatattatggatggattgtctctacaaagcttctccagtttatagcaatttcaacccaatgataaaatttaaaaattcttattccgcatgatgattgatgaaatcataatggagggggccgtttgcaagttaagaagagattaactcggctttatgtgaaaggttctcgtggcagttgaaaatggatcctcctgccacacatcgactgtaatttgattctactctaacctcgaactgaaatttttcagatgtgcactattcaccttcagccaataaaaatgctccgcaggcactttcaccccaaatggaaactctcaccgggggtttatcaactttatttcgtgggtttcacacgcccatttgtagtattattctgtaaaaacaggatgcggtgcaagtttgtcgaatcctgcccaatccaacgtttaatgttcaaaggtgaaaaaataataataataaaggactacccatttgcctcattactgcaccccattgcaatatgcaatatataataatgaaatcttcacataaaatcaaaatataatagcgtcagggatgataacgatgatgaagtctcaattctttccaggtaaaacgagttttaaggtcattgtaaccaatcccacctttattttagttcatacacatgcagaagatccaaacgttacacaaactttatatcacaaacactcaaacagccacaataacatgtagacattggattcaagtttcaacattttttgtagaacttgaaatagagcgcccgttacttcactttggatctgtgaagaagctattgacggatggcataatttcaggggccctcttccggacaaacttccttagacc

SEQ ID NO: 5 - DNA, 2000 bp, Manihot esculenta
  source 1..2000: mol_type = other DNA
tggcattgcatcagttgttttactctttcaggatattcctctacgattcttgtttcaacagagttggatgaccaaccgtattatttcttgtatgtaccaatggattagatttgtgtgcattaataataacgagctggttggctggctaggcagcagaaaatgaattaaaatggctggctttgtcgatcaagcatctttatccgttcattcccatgattggacccacttctgcctgcaacttcatcactgtccctacacaacattacctaccaccaccagcaccaccggctctggcagttccccgtttccatttctttgaaagatctttgttttcatccttttttagcttgatttttaagttatttgtgcacagttcatttcacacaagtaactagttggagattaagtccgtcagaagagtctgtttcgcattgagtttaaaggttaaaaaagtatttttcagaaaaaaatatcatgtttaaaaaaaaaagcaatatgtgttcttgacaaaattaaatttaaatattttttaaataatattttaaaaagtgtttttaaaatatgagtgctgcttaactttcctctcgttgcttgtttgaaattatcattttctagtagaatatactagaaatcttttaaattaccataatacccttagcccgcaaatctctcttattaaattcttaatctttattaaagatattataagacaatgaaaaaattattaaaatatttactgcgtgcccagctatttgtttctttttttttttttaattttttttttattgcagggttgttgcaatgacttttttcaattttccccttgtttcttacacttttttctctataaattttcaccaattcttctctttcaatcaccccacttcattgcgtttcccttctttattctccttttttgcgtttggttagtctattttctcttttattttctcttttattttctgtttgcttcttatttttcccaagatttttggtttttgagtgttctgctttagttggcttgcatacaagcctacaaggctctggagatctgctaaggactgtagttgtttcccttgatcatggcgttttactcttgctgcttatataataacttgctcaggctggcctagagaaaatttgcatatcatggcactattagtgttcaaaatttgattatcatgtaataagcggcttgatctgtggtttaaaagtttgtactcaacttacaacgtcatgatgctttttcttggtgactatgttgttctcataaggccaactttactagttttctttctaatggccgagttccttttcaacttaaatggcggaatagttcagctctaaattttctttcttgttgtcaaaatttcttatctattttctgggtcaaaatatttaacttttccacgaagaagtttcaagcttccttcttttgatggttcctgcatatgtgtttcttggagaattattctctctactctgctttattcttcttgcagggaagtctttcctttggtcaatagaaaaaaacagttctttcttctttgatcaacccattttttaaatgggaattttttttttctgctgtatcccacagctcttttaaaaataaacccattattgatattggatctttattttgcacaaggttctgattctgttgggcactctttgctaaattgtttaattatgtgtgcagtgatctatagatctttaacatttatgtgctgcattatttaagacagtgttcttagtcaacagtacactcttaagtctctaaatcaaatataaaatcaaaatggtcggcgaggtggtaggtgtagattttttttttactcttctagaatcactggttttgttgggttttgactttagggtgattatccaaatgcttatatactgatattttcagttcgttttaatatatccgtttttgtgattcttacatttgtttttgtttcatctgaactcttgcagaggttttctgatcataagct

SEQ ID NO: 6 - DNA, 779 bp, Arabidopsis thaliana
  source 1..779: mol_type = other DNA
tttccggttgaaatgggtttggtgaatatccggttcagttatatggtggccgttggaattggcttattagttgtggccgttgttgaagccgttggtattggtaagggagaagcagacttgtggctatgagtctatgaccatgactcgtgattatggagctgtcttatgaccctgaccatcaccttgatctggtggattccaatgttttcttcttcttctaataaaatattatggtcaatacaggtgctaattaagatggtaataatttcttatgtttctgtggtaaagtttgattcaattccgtagttttagataatcttatttccatacataaattttatagttttatctactttgttcttatgttttatctctagccaagagttattattattatcagaagaagaaaaaaaaaagaagcatatatacaaaaggtttaataaaatgtattatacaaggcaattatccaaattttttttgttttggtttacattgatgctctcaggatttcataaggatagagagatctattcgtatacgtgtcacgtcatgagtgggtgtttcgccaatccatgaaacgcacctagatatctaaaacacatatcaattgcgaatctgcgaagtgcgagccattaaccacgtaagcaaacaaacaatctaaaccccaaaaaaaatctatgactagccaatagcaacctcagagattgatatttcaagataagacagtatttagatttctgtattatatatagcgaaaatcgcatcaataccaaaccacc

SEQ ID NO: 7 - DNA, 1040 bp, Commellina yellow mottle virus
  source 1..1040: mol_type = other DNA
aattcatttcttaggggcttctctcggatgtacaaaaatcaagcttcagccccacatcatttcaaaaatatgtgacttctcagatgaaaaactagcaacacctgaaggtatgagaagctggttgggtatcctctcatatgctagaaattatattcaggatatcggcaaattggtgcaaccactcagacaaaagatggcaccaacaggagacaagagaatgaatccagaaacatggaagatggtaagacagataaaagaaaaggtgaaaaatctccctgatcttcagttaccacctaaagattcattcatcataatagagacggatggttgtatgactggctggggagccgtctgcaaatggaaaatgtcaaagcatgatccaagaagcaccgaaagaatttgtgcctatgctagtggatcattcaatccaataaaatcaaccatcgatgcagagattcaggcggcaatccatggcctggataaattcaaaatttattatcttgataaaaaggagctcataattcgctcagactgtgaagcaattatcaaattttacaacaagacgaacgaaaataagccgtctagagttagatggttaacattttcagatttcttaacaggtcttggaatcacagttacattcgagcacatagatggaaagcataatggcttagcagatgctctatcaagaatgataaatttcattgtggagaaaaatgatgaatctccatacaggttcacttcatcagtagaggacgcactaaaggtctgcaatgatgatcacggaagaaatttgatatccgccgtcatcaatgacatcatcacagtactgaggagatgaatacttagccatgaagtagcgtgcgaatattacctatgcctttattcgcagcgttagtggcactgaaaggcataaagtttgttcgttcttatcaaaaacgaatcttatctttgtaacttggttacccggtatgccggttcccaagctttatttccttatttaagcacttgtgtagtagcttagaaaaccaacacaacag

SEQ ID NO: 8 - DNA, 1716 bp, Solanum tuberosum
  source 1..1716: mol_type = other DNA
agctaatgctgctcttgtcactcaaaatgatggtatccctctcgtcatccagtgtttgtcaagtcctgttaggaacacagtaaggatataaacaaacattttgtggtattcttggttattgagtgcttgctgttcacttgttaaaattgcacatatacgtagtgagaaactcaactgttgagtaccattgatccgtcaatcttgtcgataactttgataaggatatttcaggcatcagacatgtcacctctatagaacttggtctttttttttaaaaataaaaataaaaatgtttggcatcatacgaacttctgttactttaggctgtatccagaataaaatgttgtttcctcattctggaattagttgttttgcacacggacgactttcgaaatttactaattgtgttcgtccgtctcaaactggctcacactttggtggtcaattttacttctcaaggtaagcaattacagaatatgaatgtcgctctcctcatatttatccgaacaataaaaaatgatatctgtttgcatatgcatgtagatcacacagggggggggggggcggggatctagggagctaaaatctaatttatattagtagatgttgaattccgttgggcttcattatgtgtttttacatattcgtttctgaaccacccccaccccggtgaaaaacattgctctgccactggctcaatgtattgacacaaatgaacttcaaactgggcaggtgaattatgctctaggagcattgtattatctatgcaatgcatcaaacaaggaagagatcttaaagccagaagtaattgatgcaatcaaaagttatgcagctgcaggtggagttagtacaagcttcagtaatttggctcaggctttcttagatcaacatgttcctcagcttaattaaaatggaggaaaccaaagattatgttgtaaaatcattttctatcctagatggtctatcggaaacaatttatttattactcctatccaattcattatattttcaaaagttatgaagtccacgaaatatgtgacgtgggtaaagacgacccatgccaagccagtgggatatagaaacaaaacatgtaataaagagaacaaataatgagtttcgaaaagaacagaagttagcataaggacgagaatcacattatcttagtgccaaccactaatcctatgtatcattctcctctttccacgtgtcatcctacacttcctttgccatcagattagatagcccggttagtacctacactgtatatcaaaaaatacgtaacaatcatccaaacatatcatcgatcaaaggatatttatcttgatgtgctttcgccgtccattgtaacgagtttggatgaatttgatatacacccactcagatatcaatatattttataaaaagaaacaaaattgaatactagtaatatctatgtagatatttattttttcaacaatcctgtaagttataaggataactcacttatatgtgacgtggataatgaagagctaggcaggcagtgagagatagaaacaaattaagcagagacgaaaaacaaatcagttaacagaatgacgaattggatcacgctttatcttagtgccaaccactgatcccatgcatcactctgctctttccacgtggcatcctctgacgtcagatcagattcctcttctttcttttttttttctgtatatatatgagcattttagtagt

SEQ ID NO: 9 - DNA, 1925 bp, Dioscorea japonica
  source 1..1925: mol_type = other DNA
agacttcgagactccttgatgatatcaaactcctccacttcatgtaaaagttccagcttcttgagtttaatggagccaaaggaatgtttagcccaatgtcttaaggacccccggagactagcaagttttttcgcaagcgtgaacgccccacatcccaccggcgtcggggtaccccaccagagcgcaacgagatcccgaaacccttccgcagtgagctaagccaactcgaataggaagggcctaggacgagagaggaaagttcgaagctcgaggcaaatgggaacatgatcagatcccaagcggggtagacttttttggataagcctggggaaaaggtcaagccactcactattgacaaggaatctatccaacttaacccagatgggatttgcttggccattggtccacgtaaactttctacctaccgaaggtggctcccccaaccccaggtccgacacaaaagtggtggcattacggaggtcctccaggtttggggtaccggaacacttacaactggggtagaatatcgcattaaaatccccacaaacaatccacgggacttcaatgccagaaccacaatccctcagctgatcccagaaggcgcacttctgatcacgagcattaggtccatatacggtagtgcatcgccacaagagattgtctagtaggtcttatttttacctctttattattttaatttactccttaaaaatacttaagtgaaattaatattcctaaaaatatagatattgcataaattgctaaagttttagaaggatttatagcatatatactcacgtacacgtatatctggaagatttttttaaatttttttatttacttttttatttttttatttttttaaaagagctaccactttagctggtttatttttattgagtacaaatcctggacaacatggttacaagaaaacaagcaactaaacattttacaaatacaaattcaagctaagttgtatggaacgaatggtaaggtcttgtactgagagagatttctgtagtgttggaatgcgagctctgtctgttgtagggaataataccctctctaacctattggtgtgtgtgttttatctactgccttctgtacggctccccatgtatgatatatggcaaccttgctttcagaaaataagacaaaatattctggtttaaataccacaaaacaaacactaccgtagacatttagaatttctccgctctcaaaagttaaaagaaaaaaaaagtctattgattctcttctatcatatgtggctgagaggagaaaaacaaagccttctaaggcttcatgccttcctaaaatctttgtatcttatttttttattttgtggattgggatactaatattaatttgttaaattatgatgacaattgcggaaaaaaaacaatttttgaagattttaaagagttatataattttataaataaacctaaaattttcattcaaaaacaggaagtgatatatataaagaccaatatattgatatacattttgcaattaagttaatacaaattccttcctatttctattgtacaagtgaataacaagctactcatttccccaaacactgcacacaggaaaaaaaaattattactctcttttgttttcttgttctcaggcgaaaaaaaaaatgaacgaaagaaaatatataccgggtcatatctctctctctcatatatatatatcttatgaaaagtacttcaagggtgtctatgctattacttgtcttatcaacttctaaatcaacacctaccacgtctcctagcacttgttatacatggtagagcatctttaattacaaaaccacatccccacacaccattaatgaaatctataaattaaagagggcacaactctaaacccactccaatctaagcattcgcaaggaattaagaaacataacaatc

SEQ ID NO: 10 - DNA, 1000 bp, Arabidopsis thaliana
  source 1..1000: mol_type = other DNA
cctacacatccacttggccacctgttgtgcaatgatgacgatgagattgcagtgtctctcgactgatgatgtagacttcacaatctcttaaaaatcccagtacttaacctcagccttaagaaaacgcagggaactacgtcctcacaaaatctttctctttgagtgtaactttcagacgcatctttggctctaaattctaaaaaggaaaattttaataggttttcataatcatgggttcactggattagcatataagtctatggttgagaaacttgagacacagactaacgaaacctctttccggatccaaaggtcctcttgtgtagttgacgtggtaaactctctaccgtcaaatttagacattagctaatctgatcaataatctcggcagctcttaaaaattaaaaattagaaatgatacgaacctcataatttttctttctcttatcaaaacaccatctttgtatctttataagcctgttgccactatttttaattgaaaatgatgcgtttgtcttatgttttctgtcctggagttcaacattatgacaatatgtatagtaaattagtgatatacaagacgtttgcaattcaagaaaaaaacttataaactaattaatattatggtccacggtgctacatattaactcttgatggttttatacatcttttctacatgctaatatgcttttaatattgtagcctaacgttataatttgttttttcttaaagaaacagtatcttgaacgaatcttaactatttctgtacttattcgattttattcgatttttatccgtgtaaaggcaaacgattattatgtaacgacgggcataaaaagagtatcgatttcctattcggagaaaaaaaaaaagataaaaattggagtgtatgtatatttcttgaattgagagtaatacaagattacggtccaggtggcggaatattattggcaaggtcacaagaacctcaaataactgatctgaagagaaatataaatcc

SEQ ID NO: 11 - DNA, 3000 bp, Cucumis melo
  source 1..3000: mol_type = other DNA
tttaatttctaatctaatttgtactagaaaagaaaaaaaaagagtcggattaattctctaccattgagtggaggatacttggatgcagttcaagttctcatctctccaatttgtcacgtgacagcggatgattaagcatatgagtaggctgcaaaagattatagacgtagaagatgatacccaatacaaaggcgtaacttttcccggatgacttttatactctttacaaaattggaagtcctattctatctacatcttaatttccagttgttataatgaagaatagtctgaaaatgatatcaattttttctttctcaataccattcaattacgttaagattattaggagctgccattattattattattattgttgttgttattattattattatgcaaccaagtttgatttgaaattgtttgccaaattttactccaatttgatgttgtttaattactttagatggtataataagaatgaagttgaatttaaagaaaagaaacaaagcttgaaagaatggaatacttaggtgtagaagatgacaacgtatttataacgtcgtatagtgtaaataaaaatgcacacatttggatgccctttatgcttcttagaggtcagactttcccacaaaggctaaggtgattcaatcgtgtgggacatcttgttctcccatttgattctcgttttcattagaccaaaattaacaaaaaaatagtaataattctattctttttaaagtttgtgatattacggtttatcctttgttaaaaaagtttatctttgaatgtaagaatttgatagaatgttgaatgaaaattaagattttgaaaagttttgctgaatttcaaataatataactctctaactttggtttaggaaaattaagtgatgacaattatctctattagaattagtattataagtgatatttgagttatgcacttgacttggtcgtgttggtaaattctttggatacagaacaaaagaagttgcatgccaagaaagatttctaatagatatggtgagatatgtggccgttggctctattggattggtggtatgttccagagaagaggagtgcgtatggatacgacctaggtggataaatgattatatgaggagatggtaattttatgaaatgtgttagagctttgatgttaatatatattttttaagtgtgttttgtgatcgatggtattagatgagttccttattaaacatgttttcttggtttttctcgaggtggggttctcaacacttggtaacatgcatcatgtccacgagatgttcttcatcttatctcttgtaatattatatatgatatctcacacaatacaggttcgtctgaaaaatctttctttatttgaaattttttaggtatttattcttgaggatttttttattcttaagtaaagtgttcatgatttgaagttagaaatataggagttatttttaagagagagtctcacactcaaagggagtctaaatatcttttttactaatttaggttgtgtaataaccttgtatttatcgataagtatcacgatgtaatcatttaactatctattaacgaaaatcttttttaggacacgttgcctcctagatagatgcaagttgtattgcaaaacttgtactctgttttttagttttttacatgttttactttagaactaaacctaagttatgttatgtgtcaaataaacttctttaaaataatattaaaacttctcaaaataataggaaaaaaaagaaaaatttcaaatttaatatatatatatatatattgtaatattagctttcattatcattgaattaaaaattgcatatacaagaatcgaataatgtggagaaagtagttttcctttttcaactttgtgtagaggctaagtctctaaaatattggcttcgactttgtacttttggatccgccaccacaatcagacaaacttccatttgatcattacctttatcgaatcaaattctttcccttccaatctgtcacaattttgaacataccatccaccttctgattttttgattctaaataaaccttattagcagagatttttaaaattagtattaaattataccaaataccctaatgaactttttcaatagtttttctattttattttttttttcttttgtgtgtatgagttttttcaccaccattagaaaacacatttgaaatatacagaaccaaattgtttaatttgaattggttttccataccatttttacaaaatacatagtataaccaaaagaactatagttttaagtagtgtataatagtttaattttaaagacaaagaactaaacaataatcattatcaaaaacactaccttaaaacagaattgaaatcaaatccatttgtttaggaatatatatatatatatatatatatataatatagtatcataatatataaaaaaaatgtcaaaatctgagattctttgatcctccctaaattgtccatttttgtcttgcctacaaacttgcaaaaaagaaaaaaaaaaaggttcatagatagaaatgacccataattgaatcataaagcaataaggatatacaaaattattatatccaagagggatgagagataatcttaaaggtgcaaaagaatcttcttattgatggaagaagagaatacaaactcttccaacttttgatcaaaatgcccataatgccctccatctcaccttaaagataggatattccaagtcatattcatcccaccaataccaatatctaaaataataagtaacaaataattacaattacaaatataaagtgcatagaaattaaacttaggggtatctataaacttaaaacaatgttccccaaggctctataaatagcctccttcccatcccttcacaactcaagcttgaaggactaaaacaagaacttgtaagcttgcccttcttattaagtccttcttgcctcccttccttcggagagaaaaaacttttgttgtttcaaaagcaccaaagtcaat

SEQ ID NO: 12 - DNA, 1008 bp, Arabidopsis thaliana
  source 1..1008: mol_type = other DNA
cctgcaggcatcaggctagtgtcaatgctatagcttgggctcctcatagctcttcccatatctgctccgctggagatgattctcaggcgttgatttgggatatatcttccatgggacagcatgttgaaggtggtctggatccgattctagcttacacagccggcgctgaggttgagcagcttcagtggtcatcttctcagcctgattgggttgccattgccttctctaacaagctgcagattctccgggtctgattatgttttacagatttctgttggatacttggatgtaaaaactcgttgctattgttgattaatgatcttttgatctctgactctaagcctcattgagtttcctttcatagtatggtctgtgtaggaactatgtaccttcaaaattgtgaataactagctttagcattggatttgattagacgtatggaagttagttttgaacagagagcatgtgacataacggggaacttgaagtagattatgactaacaccagcatttatgtttctctgccctcgagctgatggagttgcaggattttgtctgcctcaccatcgtatggaatgtgtagctttctatggtagttgttataagttttgtctccattgtccatatcactttatgggtatgtagatttgtgaaattaggcatttgtaacatcgtacagatgatgtattcttcattacttcaatacaggcatatagactctctgtccaacttaaaaggcatacaaggtctgtggtttaccaaaatcagctttactaaatcctttggccaatattctctcagccagtcctaagaacctctgctctaaaaagtcttagctttgaaagtcagacaaatggagagcagccttatcctccttttataaccgtgaccacacacacaaatgaagaagcccaaatgaaaggcccaaaaatctaaaaaataggtgaaggagggactttgtgaatatgtgatcatagaaacatgaagagacaagagagtggtaaaacc

SEQ ID NO: 13 - DNA, 1061 bp, Solanum tuberosum
  source 1..1061: mol_type = other DNA
aagctttaacgagatagaaaattataatactccgttttgttcattacttaacaaatgcaacagtatcttgtaccaaatcctctctcttttcaaacttttctatttggctgttgacagagtaatcaggatacaaaccacaagtatttaattgactcatccaccagatattatgatttatgaatcctcgaaaagcctatccattaagtcctcatctatggatatacttgacagtttcttcctatttgggtatttttttcctgccaagtggaacggagacatgttatgttgtatacgggaagctcgttaaaaaaaaaatacaataggaagaaatgtaacaaacattgaatgttgtttttaaccatccttccttttagcagtgtaccaattttgtaatagaaccatgcatctcaatcttaatactaaaaaatgcaacaaaattctagtggagggaccagtaccagtacattagatattattttttattactataataataatttaactaacacgagacataggaatgtcaagtggtagcggtaggagggagttggtttagtttttagatactaggagacagaaccggaggggcccattgcaaggcccaagttgaagtccagccgtgaatcaacaaagagagggcccataatactgttgatgagcatttccctataatacagtgtccacagttgccttccgctaagggatagccacccgctattctcttgacacgtgtcactgaaacctgctacaaataaggcaggcacctcctcattctcacactcactcacacagctcaacaagtggtaacttttactcatctcctccaattatttctgatttcatgcatgtttccctacattctattatgaatcgtgttatggtgtataaacgttgtttcatatctcatctcatctattctgattttgattctcttgcctactgtaatcggtgataaatgtgaatgcttcctcttcttcttcttcttctcagaaatcaatttctgttttgtttttgttcatctgtagcttggtagattcccctttttgtagaccacacatcac

SEQ ID NO: 14 - DNA, 1163 bp, Manihot esculenta
  source 1..1163: mol_type = other DNA
gtggaatggagattagtagcatcatagcgtgaactctcggcttttagatttagggcaaatataagggtacacttcaaaacgacgtagtataggtgatttcggttcggttcggtttaatcgaaatttttgaatctaaaattgaaccgaaccaaaatcaccgatttttcataaaattaaattcaaatcgaatcgaattaaaaataaaaccgaaccaaatttttgattcggttcagttcgatcggttatttcggtttaaaccgaatagtgctcacccctagcgagtgtaactttaatctatttttaaaattaaaattaaaattatttattttattataataattataaatattttataataaatatatcattaatttaaaatagcagtaggtataaaataattttaaaaaaatttatttattataaattcttcttaattattactttaaaattacattatttactattgaaatcgtaaattatacatttaagaattagattatcattgtctttaatattaaataaataaattaattataattaaatatgcattaatattttcatttataaaattaatttatatttaaaaataataaaatattatatttaacttggttcaaaaaaatattatatttaatatattatttaaaatttaaaattttaattttaaaaattaattaaaattatatttaaatataaatattaaatgtttaaattaaaatgtgaaataattaaaatgtggacttttttttccttataaaaatcatatatgcacggaacaaacacaaccgtccattgttcaatcaatgaattgaaatgtaggaccctggcgaagtcccaccattactaatctcaaccacaacacaagtagaggtagaggtgttgagctagtaaacttatagcatattcccactgataccctttaacctatgctaatgcaaattgccacaatgggtcccacttcacgtgctctctcccttgggaggtgcccagtatgttgggagtattttctcaaccagtaccgaagcgacacgtgtacatcaccaacagctacaatactcaccttcctccctagcaaaatctttcagtggatcatcatattatagcatatcaaatctccactccaccacaaccaccagcggaacctattttgcgcctaagcttgt

SEQ ID NO: 15 - DNA, 1501 bp, Solanum tuberosum
  source 1..1501: mol_type = other DNA
gtgttcatctgttggacaagctaataatatctatttgttgtctgaatcacaattttcccatgataaattaaagccaaaattcggcttctcacaacctgatagaatctaattttatgacttttatcaaaaaaaagaagaagatagaatctaattttatgacatttaatggtagccattccttaaaaattcccgattcaactaatccttggagttcattttctcataggtggatttcatgctggagttactgtaaatagggttggatttctacattcttgcgagagtgatctgcattccagccccctctgaagcacaggagtcagctgcatttcaaatgattacccaattacagactatggcagaccgcacccattagatttaatgtggcaaagaaacatcaactccaatatttttgaatcctattaatggctatcataccatagtactgtattagtactgtccagaagcaaaaattgttgttttagaatattacttggttgcaaccgtatcagtgctttgaacatttatcaaggccaaatacataatggtgccttgaaatcaactctagtttattgaaatgatgtctattgagcacctatatgtgttgccagtattgcccatgcttaacttagcattctgttaacaagtgttatgctggatgttcgaatcgaacatggagttttcatgttgcttagaactatatgctttcagtgttagcagtatcggaaggtaaatacagtttaaacaccaaaagtagagagaaataaggaggaatttaatctttggatttgattcgtctttcttgtgggaatggtgaactcgttaaccagctaggtgaaatgaactcaatacttagttcaaaattgtttaagttccatttttggttaagagaattgagttgtgctctctagcttttgaagatgatttaggttcaaggactataattttacacgatattagagctagatccattgataggccgcgtttcttaatgtttttggccttgtaattctgttttcacattgattgactgaatgagttgtgctctttctatatgatctcggataattctttttctcatgagctaacttaagttaggatatgagctaactgctctccaacaggcaacaacccatagaggaaccaatcagttttttctttttgtcctcctcttccttgcacgagtggacttagtaaatagaaaggaaaataacaaaaatgtataagaaacaagtcaatctcttgggtcccttaataatcaaagaaaaccaatgaatgaactccaagtgcacaaaactaagataaggcaaaggaaaataatttgtagtaagaatgctatatattgataatgttggtaataatattatctagaagcaatccacagaaggggtgccacgtgtcaacttgtaaatcacaaattgcaagtcatcactaatattattatcaagggaccatgcaaagtgaaaataaataattcatactaagtagtgagagcaatact

SEQ ID NO: 16 - DNA, 3011 bp, Ipomoea batatas
  source 1..3011: mol_type = other DNA
ggctggtttctaagacattttttggtttaatccaaacctaattacaaatattcccaacaagatcgaatgatctatggctacaaaccctatcccaacaaaaaactacatttagtacatcaaattaagtggcatgattattttattttgttcgacaaagtagcatcaaataaactacaaaaaaaactacatcattacaaaaagactaattatcaggcatcaatgttagtatatgggaggtggtgggttcgagcctcagtggaggcgttgctgtctctttgttcttcagtaggttgagagagtaatttatgaacagatactacactgtaatagagtcagtagcaatcaaaaaaaaatttgttttaataatatcctaatattatatttttctaaccagtactatgctttcggctttccagaaggcagaagcctaaaaaattcaattaagtttataaactttaatccacttgtttgagtaattgagtatctttcagaacggttgtagatttaggtgggatgacaaatggtattccaaagttcaagatatttctttttagatttaggaatttgtagtcttttaaggttagaggttacttaaaaggatgaacaaattttttatcccattctatttctaggaagaatttataatccgtacgtgtgacggctgccattaattatagtgcccattcatttttattgggaaaaagtactcatccattatttcattggcacggcaacccagttttaaatattttataacaataataacatatggaaccaaattgtaacctttatatcccacagacccacacattacacatccaataaaacttgagccaaattatatattagcgttactgagtactgactaaaatatatttttaaaatatactaaaatattattaaaaaaatattaaaatagtaaaattatattaaatagaaaatttaatttaatcaaagaataccaactaaaacgtataaaatgagaaaatataggtatataatattgatgattccttttgattttttttttatgatccgaaaattctttggccataagaagagtaaatgaacaatttaaactaagaaaataagtaagttgctcctatgtgattaatatataaagtgagatttgagctgttgatctatattattgaattagatcaacgactcaaaatgaaggataatttttttaaaaaatcgcttcctgttaatattaatgctttaaaattaagcacattaaactttaaaataatgcacctttttttttaatactattgaccttgttacatgtagtatctgaagtccaacaaagtcaacattgtccccactgaggctcaaacccgtgacctcccactagggagaatcgcttcatgccgcttgaccacaagtcctttggtaaaaataatgcaccttaaagatgtaaacttacgcatcttcgatgaactgaccactttgagcttgcaacttatacttttttgaagataagcttgtaacttattataatggtctattaacattaaaaaaaaaaaaagtttcacaatcaaattataatatttgtagccaaatgaatttaccgcgggtgtgactattcaggaatttaaatacactaaagttggaggggtagtacactcaatacactattgctcatgacttttttcttcttttttttttagattagctaatatattaatcccaaatagaaacgtttacaccaaagttcgaaaaaatgttgtgtcatttcttacagttagacacaaaaataacatttttagctaagttacagtaaacttgattggcagactgtttcacaaattgggagcttggatccttgaaggaacttactgctttcttagagtcattaatggtttggccaaacatagaaaagattagttgagcagtcttgcacactacttgagtaatcatctccattcttctacttattgacaatattctcttatgaaaaaacacacttgatcttatatcagttagggatttgaccggtttattaaaggatagcctaccaactttgttgaacgacatatcatcatatcatgattcaaaagatgctcttttttattgtcatatttgtggcacaggatgagtacagtttcgcatacaccatgatcatttttatcaaatcatactctataaaaccctgtcaaagaaaagagaggaagaaacgagaagaagaaactcatccaagaaacaagaggaacattattgctcatgattagatcgacttgaacatgtactaatgccaatctcaaattacctacataggtgtgttagacaaatatttgttaattagctgattgacttaatggatttgactagttgttaacattaattgattgtaggaaattgtttggtaaattagttgttagttgatagttgattacatgaaaattactttctcaaaaagcttatcgaaaaaactattttgaacagctttttgaattttaacattttataacaataagttgttacaaaaagctaattaatcaaatactcatatccattgtttaaccatgtcaaacaactaataataattaaataatttgtttttaaaatataagttaaatttaattgataagctaactatattaccaaacataccgtaatattttcttaaccgcggtatgggctaagatatgattgtatactatttttgttgcgagcatgattaatacagtaataccatcatttaaaagtggaaaccacattcgcagctgtttccgaaagcaaacagctaacatttgctaggttcttacttatgcattaatctgggttataaaatccccatttccatgttggtgtgaacaaccacctaaacctagcgtgttcaacaattctaccctactatcatcccccaagacttccccgaccagtaaataacccgctttcctctttcagtgatttcttcatttgactttgctatatatatatataatctgatctgctttcatctttcagtgatttcttcatttggatttcttcagaggaggagaagg

SEQ ID NO: 17 - DNA, 960 bp, Solanum tuberosum
  source 1..960: mol_type = other DNA
tgcgtattagttttagcgacgaagcactaaatcgtctttgtatactttgagtgacacatgtttagtgacgactgattgacgaaatttttttcgtctcacaaaatttttagtgacgaaacatgatttatagatgacgaaattatttgtccctcataatctaatttgttgtagtgatcattactcctttgtttgttttatttgtcatgttagttcattaaaaaaaaaatctctcttcttatcaatcctgacgtgtttaatatcataagattaaaaaatattttaatatatctttaatttaaactcacaaagtttaattttcttcgttaacttaatttgtcaaatcaggctcaaagatcgtttttcatatcggaatgaggattttatttattcttttaaaaataaagaggtgttgagctaaacaatttcaaatctcatcacacatatggggtcagccacaaaaataaagaacggttggaacggatctattatataatactaataaagaatagaaaaaggaaagtgagtgaggtgcgagggagagaatctgtttaatatcagagtcgatcatgtgtcagttttatcgatatgactttgacttcaactgagtttaagcaattctgataaggcgaggaaaatcacagtgctgaatctagaaaaatcttatacaatgtgagataaatctcaacaaaaacgttgagtccatagagggggtgtatgtgacaccccaacctcagcaaaagaaaacctcccctcaagaaggacatttgcggtgctaaacaatttcaagtctcatcacacatatatattatataatactaataaagaatagaaaaaggaaaggtaaacatcactaatgacagttgcggtgcaaagtgagtgagataataaacatcagtaatagacatcactaacttttattggttatgtcaaactcaaaataaaatttctcaacttgtttac

SEQ ID NO: 18 - DNA, 830 bp, Arabidopsis thaliana
  source 1..830: mol_type = other DNA
acttgagaagttttttttggactttgtttagctttgttgggcgttttttttttttgatcaataactttgttgggcttatgatttgtaatattttcgtggactctttagtttatttagacgtgctaactttgttgggcttatgacttgttgtaacatattgtaacagatgacttgatgtgcgactaatctttacacattaaacatagttctgttttttgaaagttcttattttcatttttatttgaatgttatatatttttctatatttataattctagtaaaaggcaaattttgcttttaaatgaaaaaaatatatattccacagtttcacctaatcttatgcatttagcagtacaaattcaaaaatttcccatttttattcatgaatcataccattatatattaactaaatccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagcgtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacctcagtcacacaaagagtaaagaagaaca

SEQ ID NO: 19 - DNA, 739 bp, Solanum lycopersicum
  source 1..739: mol_type = other DNA
aattcatggggcattacaattaattagtaatactaattttaataacactaaatcatattagtctctataggtataatatataactctggagtcactatctgtatttgggatcaacagttacaaaggtacgagtaaagtctctgatccgacaaacaatccgtatatcctaccttcccgaaatttcatgtataaaattatactaaaatattatttttcttgtattatccgattgaaaattatgcgaattcaacataaaactctaaaaaaagaaaataatgaaaattacaacttaaaagagtgtctaagttggaatcatggtattacataaagttgaggacaatataagcaagcaaacaagtactctagctatcaatttactttggactactatatgataaatatttcaacccccctcctcccccaccaaaaaaaaattaaaataacaagttgaaggactcaaaaaaaaaaaaaaaaaaaactcaaaaccaacctcaatcatacattcatatcctcttcctacccccatcttggatgagataagattaacgaggtgcttacacgtgtcacctctattgtggtgacttaaaaaaaattccaacctttcatatgtagatattaagtaattgtataatgttatcaagaaccacataacatatcaaaaaccttatcatttcattatataaaaggatagtggacatcaaaaggttcatattgaaccaaaaaaagagagaagaagcaa

SEQ ID NO: 20 - DNA, 800 bp, Arabidopsis thaliana
  source 1..800: mol_type = other DNA
gttagaattcttttgataggtttacttccattagtctgaaatggaatcttatttataagtctgcctgaaaaatcttaacaattgatattaataaaacggaaagagtaatgaatatttttaagagcatctcgaatctatctctatttttatttatatactctatttttgtatttatttaactctaactcatctctattttcatttgtaaaatagaaatttctaattttttttctaaagcaaaaaatagagatttttattttttctcttatttattttttctaacgaatttttgaggaaccattgaagttaaaatataaatctctattatattttttcctctatattcagaaagaaaataatagaaacaaatattggagatggttaaagcttttttttttgtccggataaatatcatacaatatatttatgtaaatatcaaatttaaataattttgttataactattttttaatgcgaccttatttccaaagaaagtgttaagccaaattaaatcatacatggtaaataagaggtccaaaagaccaaaagcattaaatttaaggtaatgcgtctggtttcttttctcccaaacgaaaggagccaaaagcaaccgatcaagtgcagaccagtaaccgcacattcacttcatttcccccacaagagaaaagataagataatggagttttctgccacgtggccttatcctagtggtgcgtaccgataagggtgtcaacacctttccttaatcctgtggcagtaaacgacgttatcatgaatcatggaccctttgatcatgagggc

SEQ ID NO: 21 - DNA, 1023 bp, Solanum tuberosum
  source 1..1023: mol_type = other DNA
ggagggaagaaatcttctctgtctaaaaaattgacggttgatgttataagagttttttttttttttttttttccttttacaaattgaagtgacaaaagagtgaatcacagtggatcgtgataacaaagaatgttctgagagtattgctattgtgaattttattttatatgacgtaaatggtcaaatttgcttaattttgtgaaatagaacaaatttatcctctagttggagacaaatatactcttgaactatacaaaatagaataaattttttgttatttttaaatttatcgcccaatgatattaaagatagttttttttttttttaaatgaattgaaactttcgcatagttcaatttttttttcttgcacaatatataggaaaatttaatccttctttctagttttgaattttagaaatcttttcttcctcctttctctctctaccacccattacccctttttttctccttcttccttgaacaacgccatacaattttttatttcttttttcccctcattttttttttaattccaatcaaattgtataatccaagtatagttaattttgataacatataaattataaagtaatgtgtaaataatagtactatagtataataagttaaataaatcaaactcaaataaatttagaccgctgctgtcactctctaggctctccatagccaaattttgtcgcaaccttttttgctcatttccaagaaaaaaaagaagcctactgtaatttaaaaaattccacgtgtcaatatctaaattaccaccgtcatatcgaaccaaacaatttcaaccgtcagatttacaggtacaacgtaagcaaaatagagagcattacacagaaacaacacacttcaggctgaaatttgtaggactcctccaatttgttatcgcagttcgtagaatctgaagaaagagtattattcttgattttttaatagatttttaaaaccccattaaagcaaatacgtatataattgcagcacagatacagagagggagagatact

SEQ ID NO: 22 - DNA, 2081 bp, Solanum tuberosum
  source 1..2081: mol_type = other DNA
tcaagagtcaatgatcccaatatttttaacggatactattagctttacttttattatttttgactttcctgttaagtataagatttgaaatgaatctcaatcatagtttattttattatccatatgacgttgatttaagagtcattgaacattacatgaggaaatagtacttgactgggatcttctttcatctaaactgacactaaactcttttttcttcccttctccaatatccaacatgcaattagacgatgaacgaaatgtgatgaaaaatttgataaatgagagttcaaattttaacaaaattaaataaaaaacataatcaattttttaaattttagaaatagagttattgtttaaatgatacattgaaattgcagtatatatcttatgaaataatggagataacttaaattgaccaaacattattattatttacacaaaagggggaaatagcaatttttggaccaaatattatactaaggaataggatgaaattataaaatgatttgctcgtttttttttcttctcaaaaacgaaagaaccgcacaagttgcggatctcatgagatcattacccaatgcattaggtagagtaagatccacatcactaaccttttctccgtcaatttttatttggcccatatattaaaaaaatatttatttaaaaaattagaagctaatatattattatgaagtttaatttattgttattattaactatagtaattatttcaagtatattttttaaaatattaaatttattatattcgaaagaagatgtaataaatgtatcaatctttctgtttcaatttatataattcatgttattttagtttgcctaaaaagaatgatacatttgcagtggtgacacgatttgtaaaaatttatgcgtactcattgtctatatgtatgtatcgcagcggcaagcgagatgaaagagatgcaagaagatttgttatctatttcaaaatatatatgaatcttacttagacacaatgtatatagaacaaattatatgtaatagttgaccctatatatgtggtaaaatacttgactattaggggttgtttggtagagtgtattaagaaatataatgcatatattaggtgtgtgtattagtagtaccttgtttggcacactttttcatgccatgtataactaatgcatgtgtattactaataccaaggaattctaggtattagtaataaatagcattttaacacttgcattagatcaaataattacaaaactacccttaaagcattttcattttctttgttgtcataagtttttatttttatttttatttgcttttcggtatcttttaatttgttggtgtcttaatagactttatggccttttaagtatctttttaaaaaaaatctaatttcaatataatttaaatttttttttactattgtgacaataaatttgataaaaaaaattatttgccaactttcacaaaaatattttgacgcaatagtataactatttaatactatttttttattttttatttataaaaaagatgaagagttaatgatgttttaacaaagattttttttttgatgttttagcaaaaaactttcttgcaaaggaagtgtacaaataaataaagtgtgaagggtatttttgtaaacatatattatttaatagtaattatgcaagatttattatttttaatacatcaaaccaaacaatgtataagaaataatacttgcataactaatgcacgcactactaatgcaagcattactaatgcaccatattttgtatttgttcttatacactctaccaaacgaccccttagagtgtgggtaagtaattaagttagggatttgtgggaaatggacaaatataagagagtgcaggggagtagtgcaggagattttcgtgcttttattgataaataaaaaaagggtgacatttaatttccacaagaggacgcaacacaacacacttaattcctgtgtgtgaatcaataattgacttctccaatcttcatcaataaaataattcacaatcctcactctcttatcactctcattcgaaaagctagatttgcatagagagcacaaa

SEQ ID NO: 23 - DNA, 1529 bp, Solanum tuberosum
  source 1..1529: mol_type = other DNA
aaatcattgttttattttctctttctttttacaggtataaaaggtgaaaattgaagcaagattgattgcaagctatgtgtcaccacgttattgatactttggaagaaatttttacttatatgtctttgtttaggagtaatatttgatatgttttagttagattttcttgtcatttatgctttagtataattttagttatttttattatatgatcatgggtgaattttgatacaaatatttttgtcattaaataaattaatttatcacaacttgattactttcagtgacaaaaaatgtattgtcgtagtacccttttttgttgaatatgaataattttttttattttgtgacaattgtaattgtcactacttatgataatatttagtgacatatatgtcgtcggtaaaagcaaacactttcagtgacaaaataatagatttaatcacaaaattattaaccttttttataataataaatttatccctaatttatacatttaaggacaaagtattttttttatatataaaaaatagtctttagtgacgatcgtagtgttgagtctagaaatcataatgttgaatctagaaaaatctcatgcagtgtaaaataaacctcaaaaaggacgttcagtccatagagggggtgtatgtgacaccccaacctcagcaaaagaaaacctcccttcaacaaggacatttgcggtgctaaacaatttcaagtctcatcacacatatatttattatataatactaataaagaatagaaaaggaaaggtaaacatcattaaatcgtctttgtatatttttagtgacaactgattgacgaaatctttttcgtcacacaaaatttttagtgacgaaacatgatttatagatgatgaaattatttgtccctcataatctaatttgttgtagtgatcattactcctttgtttgttttatttgtcatgttagtccattaaaaaaaaatatctctcttcttatgtacgtgaatggttggaacggatctattatataatactaataaagaatagaaaaaggaaagtgagtgaggttcgagggagagaatctgtttaatatcagagtcgatcatgtgtcaattttatcgatatgaccctaacttcaactgagtttaaccaattccgataaggcgagaaatatcatagtattgagtctagaaaaatctcatgtagtgtggggtaaacctcagcaaggacgttgagtccatagaggggggtgtatgtgacaccccaacctcagcaaaagaaaacctcccctcaagaaggacatttgcggtgctaaacaatttcaagtctcatcacacatatatatatattatataatactaataaataatagaaaaaggaaaggtaaacatcactaacgacagttgcggtgcaaactgagtgaggtaataaacatcactaacttttattggttatgtcaaactcaaagtaaaatttctcaacttgtttacgtgcctatatataccatgcttgttatatgctcaaagcaccaacaaaactttcg

SEQ ID NO: 24 - DNA, 760 bp, Arabidopsis thaliana
  source 1..760: mol_type = other DNA
gcatgcaaaatagcacaccatttatgtttatattttcaaattatttaatacatttcaatatttcataagtgtgattttttttttttttgtcaatttcataagtgtgatttgtcatttgtattaaacaattgtatcgcgcagtacaaataaacagtgggagaggtgaaaatgcagttataaaactgtccaataatttactaacacatttaaatatctaaaaagagtgtttcaaaaaaaattcttttgaaataagaaaagtgatagatatttttacgctttcgtctgaaaataaaacaataatagtttattagaaaaatgttatcaccgaaaattattctagtgccactcgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactattatataactaaaagatggaaataaaataaagtgtctttgagaatcgaacgtccatatcagtaagatagtttgtgtgaaggtaaaatctaaaagatttaagttccaaaaacagaaaataatatattacgctaaaaaagaagaaaataattaaatacaaaacagaaaaaaataatatacgacagacacgtgtcacgaagataccctacgctatagacacagctctgt

(19) United States
(12) Patent Application Publication
(10) Pub. No.: US 2024/0093220 A1
(43) Pub. Date: Mar. 21, 2024

(54) PLANT REGULATORY ELEMENTS AND USES THEREOF

(71) Applicants: Friedrich Alexander Universität Erlangen-Nürnberg, Erlangen (DE); Eidgenössische Technische Hochschule Zürich, Zurich (CH); National Chung Hsing University, Taichung (TW)

(72) Inventors: Wolfgang Zierer, Erlangen (DE); Uwe Sonnewald, Erlangen (DE); Christian Lamm, Erlangen (DE); Wilhelm Gruissem, Zurich (CH); Ravi Bodampalli Anjanappa, Zurich (CH); Shu-Heng Chang, Taichung City (TW)

(21) Appl. No.: 18/462,847

(22) Filed: Sep. 7, 2023

Related U.S. Application Data
(60) Provisional application No. 63/375,114, filed on Sep. 9, 2022.

(51) Int. Cl. C12N 15/82

(52) U.S. Cl. CPC C12N 15/8273; C12N 15/8201; C12N 15/8227; C12N 15/8245

(57) ABSTRACT

The present invention provides novel DNA molecules and constructs, including their nucleotide sequences, useful for modulating gene expression in plants and plant cells. The invention also provides transgenic plants, plant cells, plant parts, seeds, and commodity products comprising the DNA molecules operably linked to heterologous transcribable polynucleotides, along with methods of their use.

### REFERENCE TO RELATED APPLICATION

This application claims the benefit of U.S. Provisional Application No. 63/375,114, filed Sep. 9, 2022, which is hereby incorporated by reference in its entirety.

### INCORPORATION OF SEQUENCE LISTING

The sequence listing that is contained in the file named “AGOE009US_ST26.xml”, which is 56.3 KB (as measured in Microsoft Windows®) and was created on Aug. 29, 2023, is filed herewith by electronic submission and is incorporated by reference herein.

### FIELD OF THE INVENTION

The invention relates to the field of plant molecular biology and plant genetic engineering, and DNA molecules useful for modulating gene expression in plants.

### BACKGROUND

Regulatory elements are genetic elements that regulate gene activity by modulating the transcription of an operably linked transcribable polynucleotide molecule. Such elements include promoters, leaders, introns, and 3′ untranslated regions and are useful in the field of plant molecular biology and plant genetic engineering.

### SUMMARY OF THE INVENTION

The present invention provides novel gene regulatory elements for use in plants. The present invention also provides DNA constructs comprising the regulatory elements. The present invention also provides transgenic plant cells, plants, and seeds comprising the regulatory elements. The sequences may be provided operably linked to a transcribable polynucleotide molecule. In one embodiment, the transcribable polynucleotide molecule may be heterologous with respect to a regulatory sequence provided herein. A regulatory element sequence provided by the invention thus may, in particular embodiments, be defined as operably linked to a heterologous transcribable polynucleotide molecule. The present invention also provides methods of making and using the regulatory elements, the DNA constructs comprising the regulatory elements, and the transgenic plant cells, plants, and seeds comprising the regulatory elements operably linked to a transcribable polynucleotide molecule.

Thus, in one aspect, the present invention provides a DNA molecule comprising a DNA sequence selected from the group consisting of: a) a sequence with at least about 85 percent sequence identity to any of SEQ ID NOs: 1-24; b) a sequence comprising any of SEQ ID NOs: 1-24; c) a fragment of a sequence having at least 85 percent sequence identity to any of SEQ ID NOs: 1-24, wherein the fragment has gene-regulatory activity; d) a fragment of any of SEQ ID NOs: 1-24, wherein the fragment has gene-regulatory activity; and e) combinations thereof; wherein the sequence is operably linked to a heterologous transcribable polynucleotide molecule. In some embodiments, the DNA molecule is active as a promoter. In further embodiments, the DNA molecule further comprises a heterologous regulatory element. In specific embodiments, the DNA molecule comprises at least about 90 percent, at least about 95 percent, at least about 98 percent, or at least about 99 percent sequence identity to the DNA sequence of any of SEQ ID NOs: 1-24. In certain embodiments of the DNA molecule, the DNA sequence comprises a regulatory element. In some embodiments, the regulatory element comprises a promoter. In particular embodiments, the heterologous transcribable polynucleotide molecule comprises a gene of agronomic interest, such as a gene capable of providing increased yield in plants, a gene capable of providing increased root growth in plants, a gene capable of providing increased drought resistance in plants, or a gene capable of providing increased starch content in plants.

In another aspect, the invention provides a construct comprising at least one copy of a DNA molecule of provided herein, and an operably linked transcribable gene of agronomic interest. In some embodiments, the construct comprises in the 5′-3′ direction: (a) the at least one copy of said DNA molecule; (b) the operably linked transcribable gene of agronomic interest; and (c) a gene termination sequence. In further embodiments, the transcribable gene of agronomic interest comprises an open reading frame encoding a polypeptide.

The invention also provides a transgenic plant cell comprising a heterologous DNA construct provided by the invention, including a sequence of any of SEQ ID NOs: 1-24, or a fragment or variant thereof, wherein said sequence is operably linked to a heterologous transcribable polynucleotide molecule. For example, in further embodiments, the transgenic plant cell may comprise a sequence selected from the group consisting of: a) a sequence with at least 85 percent sequence identity to any of SEQ ID NOs: 1-24; b) a sequence comprising any of SEQ ID NOs: 1-24; c) a fragment of a sequence having at least 85 percent sequence identity to any of SEQ ID NOs: 1-24, wherein the fragment has gene-regulatory activity; d) a fragment of any of SEQ ID NOs: 1-24, wherein the fragment has gene-regulatory activity; and e) combinations thereof; wherein said sequence is operably linked to a heterologous transcribable polynucleotide molecule. In certain embodiments, the transgenic plant cell is a monocotyledonous plant cell. In other embodiments, the transgenic plant cell is a dicotyledonous plant cell. In particular embodiments, the transgenic plant cell is a cassava plant cell.

Further provided by the invention is a transgenic plant, or part thereof, comprising a DNA molecule as provided herein, including a DNA sequence selected from the group consisting of: a) a sequence with at least 85 percent sequence identity to any of SEQ ID NOs: 1-24; b) a sequence comprising any of SEQ ID NOs: 1-24; c) a fragment of a sequence having at least 85 percent sequence identity to any of SEQ ID NOs: 1-24, wherein the fragment has gene-regulatory activity; d) a fragment of any of SEQ ID NOs: 1-24, wherein the fragment has gene-regulatory activity; and e) combinations thereof; wherein said sequence is operably linked to a second heterologous transcribable polynucleotide molecule. In specific embodiments, the transgenic plant may be a progeny plant of any generation that comprises the DNA molecule, relative to a starting transgenic plant comprising the DNA molecule. Still further provided is a transgenic seed comprising a DNA molecule according to the invention.

In yet another aspect, the invention provides a method of producing a commodity product comprising obtaining a transgenic plant or part thereof according to the invention and producing the commodity product therefrom. In one embodiment, a commodity product of the invention is protein concentrate, protein isolate, grain, starch, seeds, meal, flour, biomass, or seed oil. In another aspect, the invention provides a commodity produced using the above method. For instance, in one embodiment the invention provides a commodity product comprising a DNA molecule as provided herein, including a DNA sequence selected from the group consisting of: a) a sequence with at least 85 percent sequence identity to any of SEQ ID NOs: 1-24; b) a sequence comprising any of SEQ ID NOs: 1-24; c) a fragment of a sequence having at least 85 percent sequence identity to any of SEQ ID NOs: 1-24, wherein the fragment has gene-regulatory activity; d) a fragment of any of SEQ ID NOs: 1-24, wherein the fragment has gene-regulatory activity; and e) combinations thereof; wherein the sequence is operably linked to a heterologous transcribable polynucleotide molecule.

In still yet another aspect, the invention provides a method of expressing a transcribable polynucleotide molecule that comprises obtaining a transgenic plant according to the invention, such as a plant comprising a DNA molecule as described herein, and cultivating a plant, wherein a transcribable polynucleotide in the DNA molecule is expressed.

Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE FIGURES

FIG. 1 shows a summary of the approximate promoter activity of ten promoters in source leaves, stem, and storage root. The relative gene expression (normalized to MeGAPDH) of different transcripts was determined and the data was used to infer the approximate activity of the promoter element controlling its expression. Field-grown cassava plants were used to sample fully exposed source leaves (in the afternoon), stem pieces at the lower end of the first branching point, and storage root material from the two thickest storage roots per plant.

FIG. 2A₁-FIG. 2I₁ show representative GUS staining pattern of at least three events from pAtCAB1::GUS, pStLS1::GUS, pAtRBCS3B::GUS, pMeGBSS1, pStSSS3, and pStSTP1 promoter-reporter plants. For pAtCAB1::GUS, FIG. 2A₁) Source leaf (Inlay=Close-up); FIG. 2B₁) Sink leaf; FIG. 2C₁) Emerging leaves; FIG. 2D₁) Petiole cross-section; FIG. 2E₁) Upper stem cross-section; FIG. 2F₁) Lower stem cross-section; FIG. 2G₁) Storage root cross-section; FIG. 2H₁) Fibrous roots; FIG. 2I₁) GUS expression levels of four pAtCAB1::GUS lines relative to three pCaMV35S::GUS lines in %. For pStLS1::GUS, FIG. 2A₂) Source leaf; FIG. 2B₂) Sink leaf; FIG. 2C₂) Petiole cross-section; FIG. 2D₂) Upper stem cross-section; FIG. 2E₂) Storage root cross-section; FIG. 2F₂) Fibrous roots. For pAtRBCS3B::GUS, FIG. 2A₃) Source leaf; FIG. 2B₃) Sink leaf; FIG. 2C₃) Petiole cross-section; FIG. 2D₃) Upper stem cross-section; FIG. 2E₃) Storage root cross-section; FIG. 2F₃) Fibrous roots. For pMeGBSS1::GUS, FIG. 2A₄) Source leaf; FIG. 2B₄) Sink leaf; FIG. 2C₄) Petiole cross-section; FIG. 2D₄) Upper stem cross-section; FIG. 2E₄) Storage root cross-section; FIG. 2F₄) Fibrous roots. For pStSSS3::GUS, FIG. 2A₅) Source leaf; FIG. 2B₅) Sink leaf; FIG. 2C₅) Petiole cross-section; FIG. 2D₅) Upper stem cross-section; FIG. 2E₅) Storage root cross-section; FIG. 2F₅) Fibrous roots. For pStSTP1::GUS, FIG. 2A₆) Source leaf; FIG. 2B₆) Sink leaf; FIG. 2C₆) Petiole cross-section; FIG. 2D₆) Upper stem cross-section; FIG. 2E₆) Storage root cross-section; FIG. 2F₆) Fibrous roots. Plants were either grown on the field at NCHU experimental station Taichung, Taiwan or in a greenhouse in Erlangen, Germany. Tissues from approximately 3-month old cassava plants were used.

FIG. 3A-FIG. 3I show representative GUS staining pattern of three pMePsbr::GUS promoter-reporter lines. FIG. 3A) Source leaf; FIG. 3B) Sink leaf; FIG. 3C) Emerging leaves; FIG. 3D) Petiole cross-section; FIG. 3E) Upper stem cross-section; FIG. 3F) Lower stem cross-section; FIG. 3G) Storage root cross-section; FIG. 3H) Fibrous roots; FIG. 3I) GUS expression levels of three pPsbR::GUS lines relative to three pCaMV35S::GUS lines in %. Bars represent mean values with standard deviation (n=4).

FIG. 4A-FIG. 4H show representative GUS staining pattern of four pAtSUC2::GUS promoter-reporter lines. FIG. 4A) Source leaf (Inlay=Close-up); FIG. 4B) Sink leaf (Inlay=Close-up); FIG. 4C) Emerging leaves; FIG. 4D) Petiole cross-section; FIG. 4E) Upper stem cross-section; FIG. 4F) Lower stem cross-section; FIG. 4G) Storage root cross-section; FIG. 4H) Fibrous roots (Inlay=Root tip).

FIG. 5A-FIG. 5H show representative GUS staining pattern of four pCmGolS1 promoter-reporter lines. FIG. 5A) Source leaf (Inlay=Close-up); FIG. 5B) Sink leaf (Inlay=Close-up); FIG. 5C) Emerging leaves; FIG. 5D) Petiole cross-section; FIG. 5E) Upper stem cross-section; FIG. 5F) Lower stem cross-section; FIG. 5G) Storage root cross-section (Inlay=Close-up); FIG. 5H) Fibrous roots (Inlay=Root tip).

FIG. 6A-FIG. 6H show representative GUS staining pattern of four pCoYMV promoter-reporter lines. FIG. 6A) Source leaf (Inlay=Close-up); FIG. 6B) Sink leaf (Inlay=Close-up); FIG. 6C) Emerging leaves; FIG. 6D) Petiole cross-section (Inlay=Close-up); FIG. 6E) Upper stem cross-section (Inlay=Close-up); FIG. 6F) Lower stem cross-section (Inlay=Close-up); FIG. 6G) Storage root cross-section (Inlay=Close-up); FIG. 6H) Fibrous roots (Inlay=Root tip).

FIG. 7A-FIG. 7H show representative GUS staining pattern of at least four pMeSWEET1-like promoter-reporter lines. FIG. 7A) Source leaf; FIG. 7B) Sink leaf; FIG. 7C) Emerging leaves; FIG. 7D) Petiole cross-section; FIG. 7E) Upper stem cross-section (Inlay=Close-up); FIG. 7F) Lower stem cross-section (Inlay=Close-up); FIG. 7G) Storage root cross-section (Inlay=Close-up); FIG. 7H) Fibrous roots (Inlay=Developing side root).

FIG. 8A-FIG. 8I show representative GUS staining pattern of at least four pMeSUS1 promoter-reporter lines. FIG. 8A) Source leaf (Inlay=Close-up); FIG. 8B) Sink leaf (Inlay=Close-up); FIG. 8C) Emerging leaves; FIG. 8D) Petiole cross-section; FIG. 8E) Upper stem cross-section; FIG. 8F) Lower stem cross-section; FIG. 8G) Storage root cross-section; FIG. 8H) Fibrous roots; FIG. 8I) GUS expression levels of three pMeSUS1::GUS lines relative to three pCaMV35S::GUS lines in %. Bars represent mean values with standard deviation (n=4).

FIG. 9A-FIG. 9I show representative GUS staining pattern of four pStPatatin Class I promoter-reporter lines. FIG. 9A) Source leaf; FIG. 9B) Sink leaf; FIG. 9C) Emerging leaves; FIG. 9D) Petiole cross-section; FIG. 9E) Upper stem cross-section; FIG. 9F) Lower stem cross-section; FIG. 9G) Storage root cross-section; FIG. 9H) Fibrous roots; FIG. 9I) GUS expression levels of three pStPatatin:GUS lines relative to three pCaMV35S::GUS lines in %. Bars represent mean values with standard deviation (n=4).

FIG. 10A-FIG. 10I show representative GUS staining pattern of at least four pStB33 promoter-reporter lines. FIG. 10A) Source leaf; FIG. 10B) Sink leaf; FIG. 10C) Emerging leaves; FIG. 10D) Petiole cross-section; FIG. 10E) Upper stem cross-section; FIG. 10F) Lower stem cross-section; FIG. 10G) Storage root cross-section; FIG. 10H) Fibrous roots (Inlay=Root tip); FIG. 10I) GUS expression levels of three pStB33::GUS lines relative to three pCaMV35S::GUS lines in %. Bars represent mean values with standard deviation (n=4).

FIG. 11A-FIG. 11I show representative GUS staining pattern of four pStGBSS1 promoter-reporter lines. FIG. 11A) Source leaf; FIG. 11B) Sink leaf; FIG. 11C) Emerging leaves; FIG. 11D) Petiole cross-section; FIG. 11E) Upper stem cross-section; FIG. 11F) Lower stem cross-section; FIG. 11G) Storage root cross-section; FIG. 11H) Fibrous roots (Inlay=Root tip); FIG. 11I) GUS expression levels of three pStGBSS1:GUS lines relative to three pCaMV35S::GUS lines in %. Bars represent mean values with standard deviation (n=4).

FIG. 12A-FIG. 12I show representative GUS staining pattern of four pMeGPT promoter-reporter lines. FIG. 12A) Source leaf; FIG. 12B) Sink leaf (Inlay=Close-up); FIG. 12C) Emerging leaves; FIG. 12D) Petiole cross-section; FIG. 12E) Upper stem cross-section; FIG. 12F) Lower stem cross-section; FIG. 12G) Storage root cross-section; FIG. 12H) Fibrous roots; FIG. 12I) GUS expression levels of three pMeGPT::GUS lines relative to three pCaMV35S::GUS lines in %. Bars represent mean values with standard deviation (n=4).

FIG. 13A-FIG. 13H show representative GUS staining pattern of pManes.14g071100 promoter-reporter lines. FIG. 13A) Source leaf; FIG. 13B) Sink leaf; FIG. 13C) Shoot apex; FIG. 13D) Petiole cross-section; FIG. 13E) Upper stem cross-section; FIG. 13F) Lower stem cross-section; FIG. 13G) Storage root cross-section; FIG. 13H) Fibrous roots.

FIG. 14A-FIG. 14H show representative GUS staining pattern of at least four pIbSRD1 promoter-reporter lines. FIG. 14A) Source leaf; FIG. 14B) Sink leaf; FIG. 14C) Emerging leaves; FIG. 14D) Petiole cross-section; FIG. 14E) Upper stem cross-section; FIG. 14F) Lower stem cross-section; FIG. 14G) Storage root cross-section (Inlay=Close-up); FIG. 14H) Fibrous roots.

FIG. 15A-FIG. 15G show cassava plant sampling positions and tissue descriptions. Different organs and organ sections were sampled from three-month-old cassava 60444 plants grown in the greenhouse and counterstained with 10% toluidine blue solution. Numbered circles, in the left figure panel, indicate the approximate sampling position of the respective samples used for GUS staining. The samples were taken from cassava plants transformed with promoter::uidA constructs and analyzed for their tissue-specific staining patterns, indicating their respective promoter activity. Note the brownish color of developing (sink) leaves in circle A, easily distinguishable from the green, fully expanded source leaf in circle B. FIG. 15A) Newly emerging leaf/sink leaf, FIG. 15B) Source leaf, FIG. 15C) Petiole cross-section, FIG. 15D) Upper stem cross-section, FIG. 15E) Lower stem cross-section, FIG. 15F) Storage root cross-section, FIG. 15G) Fibrous roots. Abbreviations: Collenchyma (Col), Cork cambium (CC), Major vein (MaV), Mesophyll (Mes), Minor vein (MiV), Parenchyma (Par), Periderm (Per), Phelloderm (Phe), Phloem Parenchyma (PPar), Phloem (Phi), Pith Parenchyma (PiPar), Pith (Pi), Protoxylem (PX), Sclerenchyma (Scl), Vascular cambium (VC), Vascular rays (VR), Xylem fiber (XF), Xylem parenchyma (XPar), Xylem vessel (XV).

FIG. 16A-FIG. 16H show representative GUS staining pattern of three pCaMV35S promoter-reporter lines. FIG. 16A) Source leaf, FIG. 16B) Sink leaf, FIG. 16C) Petiole cross-section, FIG. 16D) Upper stem cross-section, FIG. 16E) Storage root cross-section, FIG. 16H) Fibrous roots.

FIG. 17A-FIG. 17F show representative GUS staining pattern of four pStFBPase_{cyt} promoter-reporter lines. FIG. 17A) Source leaf; FIG. 17B) Sink leaf; FIG. 17C) Petiole cross-section; FIG. 17D) Upper stem cross-section; FIG. 17E) Storage root cross-section; FIG. 17F) Fibrous roots.

FIG. 18A-FIG. 18F show representative GUS staining pattern of four pDjDIO3 promoter-reporter lines. FIG. 18A) Source leaf, FIG. 18B) Sink leaf, FIG. 18C) Petiole cross-section, FIG. 18D) Upper stem cross-section, FIG. 18E) Storage root cross-section, FIG. 18F) Fibrous roots

### BRIEF DESCRIPTION OF THE SEQUENCES

SEQ ID NO: 1 is a promoter sequence of the Manihot esculenta bidirectional sugar transporter SWEET1 gene (MeSWEET1).

SEQ ID NO: 2 is a promoter sequence of the Manihot esculenta Glucose-6-Phosphate/Phosphate Translocator gene (MeGPT).

SEQ ID NO: 3 is a promoter sequence of the Manihot esculenta Photosystem II Subunit R gene (MePsbR).

SEQ ID NO: 4 is a promoter sequence of the Manihot esculenta NADH-ubiquinone reductase complex 1 MLRQ subunit (B12D) gene (Manes.14g071100).

SEQ ID NO: 5 is a promoter sequence of the Manihot esculenta Sucrose Synthase 1 gene (MeSUS1).

SEQ ID NO: 6 is a promoter sequence of the Arabidopsis thaliana Chlorophyll A/B-Binding Protein gene (AtCAB1), At1g29930.

SEQ ID NO: 7 is a promoter sequence controlling the transcription of a single viral mRNA encoding the entire Commelina yellow mottle virus (CoYMV) genome.

SEQ ID NO: 8 is a promoter sequence of the Solanum tuberosum Cytosolic Fructose-1,6-Bisphosphatase gene (StFBPase_{cyt}).

SEQ ID NO: 9 is a promoter sequence of the Dioscorea japonica Dioscorin 3 Small Subunit gene (DjDio3).

SEQ ID NO: 10 is a promoter sequence of the Arabidopsis thaliana Fructose-Bisphosphate Aldolase 2 gene (AtFBA2), AT4G38970.

SEQ ID NO: 11 is a promoter sequence of the Cucumis melo Galactinol Synthase1 gene (CmGolS1).

SEQ ID NO: 12 is a promoter sequence of the Arabidopsis thaliana Glyceraldehyde 3-Phosphate Dehydrogenase Subunit A gene (AtGAPA), AT3G26650.

SEQ ID NO: 13 is a promoter sequence of the Solanum tuberosum Granule-Bound Starch Synthase1 gene (StGBSS1).

SEQ ID NO: 14 is a promoter sequence of the Manihot esculenta Granule-Bound Starch Synthase1 gene (MeGBSS1).

SEQ ID NO: 15 is a promoter sequence of the Solanum tuberosum Leaf-Specific 1 gene (StLS1), X04753.1.

SEQ ID NO: 16 is a promoter sequence of the Ipomoea batatas Mads-Box Protein SRD1 gene (IbSRD1).

SEQ ID NO: 17 is a promoter sequence of the Solanum tuberosum Patatin Class 1 gene (StPat).

SEQ ID NO: 18 is a promoter sequence of the Arabidopsis thaliana Ribulose Bisphosphate Carboxylase Small Subunit 1A gene (AtRBCS1A), AT1G67090.

SEQ ID NO: 19 is a promoter sequence of the Solanum lycopersicum Ribulose Bisphosphate Carboxylase Small Subunit 2 gene (SlRBCS2), X66069.1.

SEQ ID NO: 20 is a promoter sequence of the Arabidopsis thaliana Ribulose Bisphosphate Carboxylase Small Subunit 3 gene (AtRBCS3B), At5g38410.

SEQ ID NO: 21 is a promoter sequence of the Solanum tuberosum Soluble Starch Synthase 3 gene (StSSS3).

SEQ ID NO: 22 is a promoter sequence of the Solanum tuberosum Starch Phosphorylase 1 gene (StSTP1), X73684.1.

SEQ ID NO: 23 is a promoter sequence of the Solanum tuberosum B33 gene (StB33).

SEQ ID NO: 24 is a promoter sequence of the Arabidopsis thaliana Sucrose-Proton Symporter 2 gene (AtSUC2).

### DETAILED DESCRIPTION OF THE INVENTION

There is an urgent need to increase agricultural output in Sub-Saharan Africa to combat hunger and malnutrition. According to the latest Food and Agricultural Organization of the United Nations (FAO) report, 239 million people in Sub-Saharan Africa suffer from chronic hunger; and 399 million people are at least moderately food insecure. Furthermore, the food insecurity situation in Sub-Saharan Africa has grown worse in recent years, mainly due to changing climate, conflicts, and economic slowdowns.

The starchy crop cassava (Manihot esculenta) is a key staple food in Sub-Saharan Africa, providing millions of people with food. Due to the limited amount of arable land available for farming in this region, improving cassava yield and/or nutritional content through plant genetic engineering represents an important approach to support food security in Sub-Saharan Africa, especially for smallholder farmers with limited resources for agricultural inputs, like industrial fertilizer. Successful implementation of many plant genetic engineering concepts depends on the availability of the right spatiotemporal expression tools; however, well-characterized cassava promoters are scarce in the public domain. Additionally, yield as well as other beneficial traits may be polygenic, depending on the interaction of many genes. Flux through biochemical pathways is often coordinated with that of competing pathways, therefore, effective metabolic engineering will only be achieved by controlling multiple genes of the same, or interconnected, pathways. Thus, a larger number of novel, well-characterized promoters are needed to support cassava biotechnology approaches, especially for applications requiring expression of multiple genes in different tissues or subcellular compartments. In particular, promoter sequences specific for autotrophic tissues, promoters specific for heterotrophic tissues (e.g. phloem or storage parenchyma), or promoters with very cell-specific expression patterns, will be valuable.

The present disclosure, therefore, provides polynucleotide molecules having beneficial gene regulatory activity from plant species. The design, construction, and use of these polynucleotide molecules are provided by the invention. The nucleotide sequences of these polynucleotide molecules are provided herein, e.g. SEQ ID NOs: 1-24. These polynucleotide molecules are, for instance, capable of affecting the expression of an operably linked transcribable polynucleotide molecule in plant tissues, and therefore selectively regulating gene expression, or activity of an encoded gene product, in transgenic plants. The present invention also provides methods of modifying, producing, and using the same. The invention also provides compositions, transformed host cells, transgenic plants, and seeds containing the promoters and/or other disclosed nucleotide sequences, and methods for preparing and using the same.

The following definitions and methods are provided to better define the present invention and to guide those of ordinary skill in the art in the practice of the present invention. Unless otherwise noted, terms are to be understood according to conventional usage by those of ordinary skill in the relevant art.

### DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

As used herein, a “recombinant DNA molecule” is a DNA molecule comprising a combination of DNA molecules that would not naturally occur together without human intervention. For instance, a recombinant DNA molecule may be a DNA molecule that is comprised of at least two DNA molecules heterologous with respect to each other, a DNA molecule that comprises a DNA sequence that deviates from DNA sequences that exist in nature, or a DNA molecule that has been incorporated into a host cell's DNA by genetic transformation or gene editing.

The polynucleotides of the invention may be synthetic nucleotide sequences. A “synthetic nucleotide sequence” is a nucleotide sequence that is not known to occur in nature or that is not naturally occurring. In some embodiments, the polynucleotide shares little or no extended homology to natural sequences. Extended homology in this context generally refers to 100% sequence identity extending beyond about 25 nucleotides of contiguous sequence.

Any number of methods well known to those skilled in the art can be used to isolate and manipulate a DNA molecule, or fragment thereof, disclosed in the present invention. For example, PCR (polymerase chain reaction) technology can be used to amplify a particular starting DNA molecule and/or to produce variants of the original molecule. DNA molecules, or fragments thereof, can also be obtained by other techniques such as by directly synthesizing the fragment by chemical means, as is commonly practiced by using an automated oligonucleotide synthesizer.

As used herein, the term “sequence identity” refers to the extent to which two optimally aligned polynucleotide sequences or two optimally aligned polypeptide sequences are identical. An optimal sequence alignment is created by manually aligning two sequences, e.g. a reference sequence and another sequence, to maximize the number of nucleotide matches in the sequence alignment with appropriate internal nucleotide insertions, deletions, or gaps. As used herein, the term “reference sequence” refers to a sequence provided as the polynucleotide sequences of SEQ ID NOs: 1-24.

As used herein, the term “percent sequence identity” or “percent identity” or “% identity” is the identity fraction times 100. The “identity fraction” for a sequence optimally aligned with a reference sequence is the number of nucleotide matches in the optimal alignment, divided by the total number of nucleotides in the reference sequence, e.g. the total number of nucleotides in the full length of the entire reference sequence. Thus, one embodiment of the invention is a DNA molecule comprising a sequence that when optimally aligned to a reference sequence, provided herein as SEQ ID NOs: 1-24, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments, such sequences may be defined as having gene-regulatory activity or having the activity of the reference sequence.

### Regulatory Elements

A regulatory element is a DNA molecule having gene regulatory activity, i.e. one that has the ability to affect the transcription and/or translation of an operably linked transcribable polynucleotide molecule. The term “gene regulatory activity” thus refers to the ability to affect the expression pattern of an operably linked transcribable polynucleotide molecule by affecting the transcription and/or translation of that operably linked transcribable polynucleotide molecule. As used herein, a transcriptional regulatory sequence may be comprised of operably linked expression elements, such as enhancers, promoters, leaders, such as 5′-untranslated regions or part thereof, introns, 3′-untranslated regions or part thereof, terminators, transcription termination regions (or 3′ UTRs), or chromatin control elements that function in plants can therefore be useful for modifying plant phenotypes through genetic engineering. Thus, a transcriptional regulatory sequence may be comprised, for instance, of a promoter operably linked 5′ to a leader sequence, which is in turn operably linked 5′ to an intron sequence. Leaders and introns may positively affect transcription of an operably linked transcribable polynucleotide molecule as well as translation of the resulting transcribed RNA. The pre-processed RNA molecule comprises leaders and introns, which may affect the post-transcriptional processing of the transcribed RNA and/or the export of the transcribed RNA molecule from the cell nucleus into the cytoplasm. Following post-transcriptional processing of the transcribed RNA molecule, the leader sequence may be retained as part of the final messenger RNA and may positively affect the translation of the messenger RNA molecule.

Regulatory elements such as promoters, enhancers, leaders, such as 5′-untranslated regions or part thereof, introns, 3′-untranslated regions or part thereof, transcription termination regions (or 3′ UTRs), or chromatin control elements are DNA molecules that have gene regulatory activity and play an integral part in the overall expression of genes in living cells. The term “regulatory element” refers to a DNA molecule having gene regulatory activity, i.e. one that has the ability to affect the transcription and/or translation of an operably linked transcribable polynucleotide molecule. Isolated regulatory elements, such as promoters and leaders that function in plants are therefore useful for modifying plant phenotypes through the methods of genetic engineering.

It is recognized that polynucleotides of the present invention can comprise a plurality of regulatory elements such as, for example, a promoter and an enhancer. It is further recognized that some genetic regulatory elements act in concert with other genetic regulatory elements to control the regulation of an operably linked gene of interest. Moreover, it is recognized that some genetic regulatory elements such as, for example, a promoter or enhancer, can be separated from the transcribed region of a gene of interest by 1, 2, 3, or more kilobases of DNA.

The present invention also provides methods for controlling gene expression. “Controlling gene expression” refers to controlling the expression of an RNA transcript, and can further encompass translation of the transcript, or even an activity or function of the encoded protein. Controlling gene expression can include affecting one or more of RNA transcription, processing, turnover, and/or translation.

The genetic regulatory elements as disclosed herein can be implemented as regulatory sequences to control gene expression in a “desired manner.” The desired manner of gene expression can be temporally, spatially, or any combination thereof in a target organism including, but not limited to, constitutive expression, tissue-preferred expression, and organ-preferred expression. The desired manner of gene expression can also be expression in response to biotic stress (e.g., fungal, bacterial, and viral pathogens, insects, herbivores, and the like) and/or abiotic stress (e.g., wounding, drought, cold, heat, high nutrient levels, low nutrient levels, metals, light, herbicides and other synthetic chemicals, and the like).

Regulatory elements may be characterized by their expression pattern effects (qualitatively and/or quantitatively), e.g. positive or negative effects and/or constitutive or other effects such as by their temporal, spatial, developmental, tissue, environmental, physiological, pathological, cell cycle, and/or chemically responsive expression pattern, and any combination thereof, as well as by quantitative or qualitative indications. A promoter is useful as a regulatory element for modulating the expression of an operably linked transcribable polynucleotide molecule.

As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as a dsRNA, a tRNA, an rRNA, a miRNA, and the like.

As used herein, the term “protein expression” is any pattern of translation of a transcribed RNA molecule into a protein molecule. Protein expression may be characterized by its temporal, spatial, developmental, or morphological qualities as well as by quantitative or qualitative indications.

### Promoters

A regulatory element, such as a promoter of the invention, may be operably linked to a transcribable DNA molecule that is heterologous with respect to the regulatory element. As used herein, the term “heterologous” refers to the combination of two or more DNA molecules when such a combination is not normally found in nature. For example, the two DNA molecules may be derived from different species, and/or the two DNA molecules may be derived from different genes, e.g., different genes from the same species or the same genes from different species. A regulatory element is thus heterologous with respect to an operably linked transcribable DNA molecule if such a combination is not normally found in nature, i.e., the transcribable DNA molecule does not naturally occur operably linked to the regulatory element.

The transcribable DNA molecule may generally be any DNA molecule for which expression of a transcript is desired. Such expression of a transcript may result in translation of the resulting mRNA molecule, and thus protein expression. Alternatively, for example, a transcribable DNA molecule may be designed to ultimately cause decreased expression of a specific gene or protein. In one embodiment, this may be accomplished by using a transcribable DNA molecule that is oriented in the antisense direction. One of ordinary skill in the art is familiar with using such antisense technology. Any gene may be negatively regulated in this manner, and, in one embodiment, a transcribable DNA molecule may be designed for suppression of a specific gene through expression of a dsRNA, siRNA, or miRNA molecule.

In some embodiments, the present disclosure provides polynucleotides containing promoters and/or enhancers. “Promoter” refers to a nucleotide sequence that is capable of controlling the expression of an operably linked coding sequence or other sequence encoding an RNA that is not necessarily translated into a protein. Thus, the polynucleotide may comprise proximal promoter elements as well as more distal upstream elements, the latter elements often referred to as enhancers. An “enhancer” refers to a DNA sequence that can stimulate promoter activity and may be an innate element of the promoter or a heterologous element inserted to enhance the level or tissue-specificity of a promoter. It is understood by those skilled in the art that different promoters may direct the expression of a gene in different tissues or cell types, at different stages of development, or in response to different environmental conditions. It is further recognized that because in most cases the exact boundaries of regulatory sequences have not been completely defined, nucleic acid fragments of some variation may have identical or similar promoter activity.

Promoters that cause a gene to be expressed in most cell types of an organism and at most times are commonly referred to as “constitutive promoters”. Expression of a gene in most cell types of an organism and at most times is referred to herein as “constitutive gene expression” or “constitutive expression”.

In some embodiments, the regulatory element is an expression-enhancing intron. An “expression-enhancing intron” or “enhancing intron” is an intron that is capable of causing an increase in the expression of a gene to which it is operably linked. While the present invention is not known to depend on a particular biological mechanism, it is believed that the expression-enhancing introns of the present invention enhance expression through intron mediated enhancement (IME). It is recognized that naturally occurring introns that enhance expression through IME are typically found within 1 Kb of the transcription start site of their native genes (see, Rose et al. (2008) Plant Cell 20:543-551). Such introns are usually the first intron, whether the first intron is in the 5′ UTR or the coding sequence, and need to be in a transcribed region. Introns that enhance expression solely through IME do not enhance gene expression when they are inserted into a non-transcribed region of a gene, such as, for example, a promoter. That is, they do not function as transcriptional enhancers. Unless stated otherwise or apparent from the context, the expression-enhancing introns of the present invention are capable of enhancing gene expression when they are found in a transcribed region of a gene but not when they occur in a non-transcribed region such as, for example, a promoter.

In some embodiments, the promoter is a plant promoter. A “plant promoter” is a promoter capable of initiating transcription in plant cells whether or not its origin is a plant cell, e.g., it is well known that Agrobacterium promoters are functional in plant cells. Thus, plant promoters include promoter DNA obtained from plants, plant viruses and bacteria such as Agrobacterium and Bradyrhizobium bacteria, and synthetic promoters capable of initiating transcription in plant cells. A plant promoter can be a constitutive promoter, a non-constitutive promoter, an inducible promoter, a repressible promoter, a tissue specific promoter (e.g., a root specific promoter, a stem specific promoter, a leaf specific promoter), a tissue preferred promoter (e.g., a root preferred promoter, a stem preferred promoter, a leaf preferred promoter), a cell type specific or preferred promoter (e.g., a meristem cell specific/preferred promoter), or many other types. In some embodiments, the variant polynucleotides or fragments described herein include additional known cis-acting sequences to drive expression of a transcribed gene in a desired manner.

In some embodiments, the promoter is a constitutive promoter. A “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or scoreable markers, allowing easy detection and quantification; high level of production of a transcription factor that is part of a regulatory transcription system; production of compounds that requires ubiquitous activity in the plant; and production of compounds that are required during all stages of plant development. For illustration, constitutive promoters can include CaMV 19S promoter, CaMV 35S promoter (U.S. Pat. Nos. 5,352,605; 5,530,196 and 5,858,742), opine promoters, ubiquitin promoter, actin promoter, alcohol dehydrogenase promoter, etc. In some embodiments, the synthetic promoter prepared as described herein, is used to drive expression of a heterologous sequence, while CaMV 35S promoter is used to drive expression of a second sequence.

In some embodiments, the promoter is a non-constitutive promoter. A “non-constitutive promoter” is a promoter which is active under certain conditions, in certain types of cells, and/or during certain development stages. For example, tissue specific, tissue preferred, cell type specific, cell type preferred, inducible promoters, and promoters under developmental control are non-constitutive promoters. Examples of promoters under developmental control include promoters that preferentially initiate transcription in certain tissues, such as stems, leaves, roots, or seeds.

In some embodiments, the promoter is an inducible or a repressible promoter. An “inducible” or “repressible” promoter is a promoter which is under chemical or environmental factor control. Examples of environmental conditions that may affect transcription by inducible promoters include cold, heat, drought, certain chemicals, or the presence of light.

In some embodiments, the promoter is a tissue-specific promoter. A “tissue-specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large number of tissue-specific promoters isolated from particular plants and tissues found in both scientific and patent literature. Non-limiting examples of known tissue-specific promoters can include beta-amylase gene or barley hordein gene promoters (for seed gene expression), tomato pz7 and pz130 gene promoters (for ovary gene expression), tobacco RD2 gene promoter (for root gene expression), banana TRX promoter and melon actin promoter (for fruit gene expression), and embryo specific promoters, e.g., a promoter associated with an amino acid permease gene (AAP1), an oleate 12-hydroxylase:desaturase gene from Lesquerella fendleri (LFAH12), an 2S2 albumin gene (2S2), a fatty acid elongase gene (FAE1), or a leafy cotyledon gene (LEC2).

In some embodiments, the promoter is a tissue-preferred promoter. A “tissue-preferred” promoter is a promoter that initiates transcription mostly, but not necessarily entirely or solely in certain tissues.

In some embodiments, the promoter is a cell-type-specific promoter. A “cell-type-specific” promoter is a promoter that primarily drives expression in certain cell types in one or more organs, for example, vascular cells in roots, leaves, stalk cells, and stem cells.

In some embodiments, the promoter is a cell-type-preferred promoter. A “cell-type preferred” promoter is a promoter that primarily drives expression mostly, but not necessarily entirely or solely in certain cell types in one or more organs, for example, vascular cells in roots, leaves, stalk cells, and stem cells.

In some embodiments, the promoter is a root-specific promoter. A “root-specific” promoter is a promoter that initiates transcription only in root tissues.

In some embodiments, the promoter is a root-preferred promoter. A “root-preferred” promoter is a promoter that initiates transcription mostly, but not necessarily entirely or solely in root tissues.

In some embodiments, the present invention provides for methods to obtain inbred plants comprising the polynucleotide sequences. As used herein, the term “inbred” or “inbred plant” is used in the context of the present invention. This also includes any single gene conversions of that inbred. The phrase “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

As used herein, the term “promoter” or a molecule having “promoter activity” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, that is a promoter produced through the fusion of two or more heterologous DNA molecules. Promoters useful in practicing the present invention include SEQ ID NO: 1-24, or fragments, variants, functional fragments or variants thereof, or combinations thereof. In specific embodiments of the invention, such molecules and any variants or derivatives thereof as described herein, are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” which provides a basal level of transcription and is comprised of a TATA box or equivalent sequence for recognition and binding of the RNA polymerase II complex for initiation of transcription.

In one embodiment, fragments are provided of a promoter sequence disclosed herein. Promoter fragments may comprise promoter activity, as described above, and may be useful alone or in combination with other promoters and promoter fragments, such as in constructing chimeric promoters. In specific embodiments, fragments of a promoter are provided comprising at least about 50, 95, 150, 250, 500, 750, 1000, 1250, 1500, 1750, or at least about 2000 contiguous nucleotides, or longer, of any of SEQ ID NOs: 1-24 or a polynucleotide molecule having promoter activity disclosed herein. Fragments of SEQ ID NOs: 1-24 may have the activity of the reference promoter sequence.

Compositions derived from any of the promoters presented as SEQ ID NO: 1-24, such as internal or 5′ deletions, for example, can be produced using methods known in the art to improve or alter expression, including by removing elements that have either positive or negative effects on expression; duplicating elements that have positive or negative effects on expression; and/or duplicating or removing elements that have tissue or cell specific effects on expression. Compositions derived from any of the promoters presented as SEQ ID NO: 1-24 comprised of 3′ deletions in which the TATA box element or equivalent sequence thereof and downstream sequence is removed can be used, for example, to make enhancer elements. Further deletions can be made to remove any elements that have positive or negative; tissue specific; cell specific; or timing specific (such as, but not limited to, circadian rhythms) effects on expression. Any of the promoters presented as SEQ ID NO: 1-24 and fragments or enhancers derived therefrom can be used to make chimeric transcriptional regulatory element compositions comprised of any of the promoters presented as SEQ ID NO: 1-24 and the fragments or enhancers derived therefrom operably linked to other enhancers and promoters. The efficacy of the modifications, duplications, or deletions described herein on the desired expression aspects of a particular transgene may be tested empirically in stable and transient plant assays, such as those described in the working examples herein, so as to validate the results, which may vary depending upon the changes made and the goal of the change in the starting molecule.

As used herein, the term “leader” refers to a DNA molecule isolated from the untranslated 5′ region (5′ UTR) of a genomic copy of a gene and defined generally as a nucleotide segment between the transcription start site (TSS) and the protein coding sequence start site. Alternately, leaders may be synthetically produced or manipulated DNA elements. A leader can be used as a 5′ regulatory element for modulating expression of an operably linked transcribable polynucleotide molecule. Leader molecules may be used with a heterologous promoter or with their native promoter. Promoter molecules of the present invention may thus be operably linked to their native leader or may be operably linked to a heterologous leader. Leaders known in the art may be useful in practicing the present invention. The leader sequences (5′ UTR) may be comprised of regulatory elements or may adopt secondary structures that can have an effect on transcription or translation of a transgene. Leader sequences known in the art can be used in accordance with the invention to make chimeric regulatory elements that affect transcription or translation of a transgene. In addition, leader sequences can be used to make chimeric leader sequences that affect transcription or translation of a transgene.

The introduction of a foreign gene into a new plant host does not always result in a high expression of the incoming gene. Furthermore, if dealing with complex traits, it is sometimes necessary to modulate several genes with spatially or a temporarily different expression pattern. Introns can principally provide such modulation. However multiple uses of the same intron in one plant have shown to exhibit disadvantages. In those cases, it is necessary to have a collection of basic control elements for the construction of appropriate recombinant DNA elements.

In accordance with the invention, a promoter or promoter fragment may be analyzed for the presence of known promoter elements, i.e. DNA sequence characteristics, such as a TATA-box and other known transcription factor binding site motifs. Identification of such known promoter elements may be used by one of skill in the art to design variants of the promoter having a similar expression pattern to the original promoter.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide sequence. Unlike promoters, enhancer elements do not usually include a transcription start site (TSS) or TATA box or equivalent sequence. A promoter may naturally comprise one or more enhancer elements that affect the transcription of an operably linked polynucleotide sequence. An isolated enhancer element may also be fused to a promoter to produce a chimeric promoter cis-element, which confers an aspect of the overall modulation of gene expression. A promoter or promoter fragment may comprise one or more enhancer elements that effect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element may function to bind transcription factors that regulate transcription. Some enhancer elements bind more than one transcription factor, and transcription factors may interact with different affinities with more than one enhancer domain. Enhancer elements can be identified by a number of techniques, including deletion analysis, i.e. deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-element motifs or enhancer elements as a target sequence or target motif with conventional DNA sequence comparison methods, such as BLAST. The fine structure of an enhancer domain can be further studied by mutagenesis (or substitution) of one or more nucleotides or by other conventional methods. Enhancer elements can be obtained by chemical synthesis or by isolation from regulatory elements that include such elements, and they can be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed by the present invention. Enhancer sequences derived from the CaMV can also be utilized (U.S. Pat. Nos. 5,164,316; 5,196,525; 5,322,938; 5,530,196; 5,352,605; 5,359,142; and 5,858,742 for example).

In plants, the inclusion of some introns in gene constructs leads to increased mRNA and protein accumulation relative to constructs lacking the intron.

This effect has been termed “intron mediated enhancement” (IME) of gene expression (Mascarenhas et al., (1990) Plant Mol. Biol. 15:913-920). Introns known to stimulate expression in plants have been identified in maize genes (e.g. tubA1, Adh1, Sh1, Ubi1 (Jeon et al. (2000) Plant Physiol. 123:1005-1014; Callis et al. (1987) Genes Dev. 1:1183-1200; Vasil et al. (1989) Plant Physiol. 91:1575-1579; Christiansen et al. (1992) Plant Mol. Biol. 18:675-689) and in rice genes (e.g. salt, tpi: McElroy et al., Plant Cell 2:163-171 (1990); Xu et al., Plant Physiol. 106:459-467 (1994)). Similarly, introns from dicotyledonous plant genes like those from petunia (e.g. rbcS), potato (e.g. st-ls1), and Arabidopsis thaliana (e.g. ubq3 and pat1) have been found to elevate gene expression rates (Dean et al. (1989) Plant Cell 1:201-208; Leon et al. (1991) Plant Physiol. 95:968-972; Norris et al. (1993) Plant Mol Biol 21:895-906; Rose and Last (1997) Plant J. 11:455-464). It has been shown that deletions or mutations within the splice sites of an intron reduce gene expression, indicating that splicing might be needed for IME (Mascarenhas et al. (1990) Plant Mol Biol. 15:913-920; Clancy and Hannah (2002) Plant Physiol. 130:918-929). However, that splicing per se is not required for a certain IME in dicotyledonous plants has been shown by point mutations within the splice sites of the pat1 gene from A. thaliana (Rose and Beliakoff (2000) Plant Physiol. 122:535-542).

Enhancement of gene expression by introns is not a general phenomenon because some intron insertions into recombinant expression cassettes fail to enhance expression (e.g. introns from dicot genes (rbcS gene from pea, phaseolin gene from bean and the stls-1 gene from Solanum tuberosum) and introns from maize genes (adh1 gene the ninth intron, hsp81 gene the first intron)) (Chee et al. (1986) Gene 41:47-57; Kuhlemeier et al. (1988) Mol Gen Genet 212:405-411; Mascarenhas et al. (1990) Plant Mol. Biol. 15:913-920; Sinibaldi and Mettler (1992) In W E Cohn, K Moldave, eds, Progress in Nucleic Acid Research and Molecular Biology, Vol 42. Academic Press, New York, pp 229-257; Vancanneyt et al. 1990 Mol. Gen. Genet. 220:245-250). Therefore, not each intron can be employed in order to manipulate the gene expression level of non-endogenous genes or endogenous genes in transgenic plants. What characteristics or specific sequence features must be present in an intron sequence in order to enhance the expression rate of a given gene is not known in the prior art and therefore from the prior art it is not possible to predict whether a given plant intron, when used heterologously, will cause enhancement of expression at the DNA level or at the transcript level (IME).

As used herein, the term “chimeric” refers to a single DNA molecule produced by fusing a first DNA molecule to a second DNA molecule, where neither first nor second DNA molecule would normally be found in that configuration, i.e. fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; an example would be the fusion of a promoter to an enhancer element. Thus, the design, construction, and use of chimeric promoters according to the methods disclosed herein for modulating the expression of operably linked transcribable polynucleotide molecules are encompassed by the present invention.

As used herein, the term “variant” refers to a second DNA molecule that is in composition similar, but not identical to, a first DNA molecule and yet the second DNA molecule still maintains the general functionality, i.e. same or similar expression pattern, of the first DNA molecule. A variant may be a shorter or truncated version of the first DNA molecule and/or an altered version of the sequence of the first DNA molecule, such as one with different restriction enzyme sites and/or internal deletions, substitutions, and/or insertions. A “variant” can also encompass a regulatory element having a nucleotide sequence comprising a substitution, deletion, and/or insertion of one or more nucleotides of a reference sequence, wherein the derivative regulatory element has more or less or equivalent transcriptional or translational activity than the corresponding parent regulatory molecule. The regulatory element “variants” will also encompass variants arising from mutations that naturally occur in bacterial and plant cell transformation. In the present invention, a polynucleotide sequence provided as SEQ ID NOs: 1-24 may be used to create variants that are in composition similar, but not identical to, the polynucleotide sequence of the original regulatory element, while still maintaining the general functionality, i.e. same or similar expression pattern, of the original regulatory element. Production of such variants of the present invention is well within the ordinary skill of the art in light of the disclosure and is encompassed within the scope of the present invention. Chimeric regulatory element “variants” comprise the same constituent elements as a reference sequence but the constituent elements comprising the chimeric regulatory element may be operatively linked by various methods known in the art, such as, restriction enzyme digestion and ligation, ligation independent cloning, modular assembly of PCR products during amplification, or direct chemical synthesis of the regulatory element as well as other methods known in the art. The resulting chimeric regulatory element “variant” can be comprised of the same, or variants of the same, constituent elements of the reference sequence but differ in the sequence or sequences that comprise the linking sequence or sequences which allow the constituent parts to be operatively linked. In the present invention, a polynucleotide sequence provided as SEQ ID NOs: 1-24 provide a reference sequence wherein the constituent elements that comprise the reference sequence may be joined by methods known in the art and may comprise substitutions, deletions and/or insertions of one or more nucleotides or mutations that naturally occur in bacterial and plant cell transformation.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell. The term includes an expression cassette isolated from any of the aforementioned molecules.

### Expression Cassettes

The polynucleotide of the present invention can be provided in expression cassettes for expression of a gene of interest in the plant or other organism or host cell of interest. It is recognized that the polynucleotide of the present invention and expression cassettes comprising them can be used for the expression in both human and non-human host cells including, but not limited to, host cells from plants, animals, fungi, and algae. In one embodiment of the invention, the host cells are human host cells or a host cell line that is incapable of differentiating into a human being.

The expression cassette can include 5′ and 3′ regulatory sequences operably linked to the gene of interest to be expressed. “Operably linked” is intended to mean a functional linkage between two or more elements. For example, an operable linkage between one or more genetic regulatory elements and a gene of interest is functional link between the gene of interest and the one or more genetic regulatory elements that allows for expression of the gene of interest. Operably linked elements may be contiguous or non-contiguous. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes. The expression cassette can include, in the 5′-3′ direction of transcription, a transcriptional and translational initiation region (i.e., a promoter), polynucleotide to be expressed, and a transcriptional and translational termination region (i.e., termination region) functional in plants or other organism or host cell. The regulatory regions (i.e., promoters, transcriptional regulatory regions, and translational termination regions) and/or the polynucleotide to be expressed may be native/analogous to the host cell or to each other. The promoter may be provided by the polynucleotide of the invention in some embodiments.

Where appropriate, the genes of interest may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. A selectable marker gene can be positively or negatively selectable. For positive selection, a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of nontransformed plant cells and reducing the possibility of chimeras. Non-limiting exemplary marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, sulfonylurea, glyphosate, glufosinate, L-phosphinothricin, triazine, benzonitrile and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng. 85:610-9 and Fetter et al. (2004) Plant Cell 16:215-28), cyan florescent protein (CYP) (Bolte et al. (2004) J. Cell Science 117:943-54 and Kato et al. (2002) Plant Physiol. 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) PNAS 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) PNAS 86:5400-5404; Fuerst et al. (1989) PNAS 86:2549-2553; Deuschle et al. (1990) Science 248:480-483; Gossen (1993) Ph.D. Thesis, University of Heidelberg; Reines et al. (1993) PNAS 90:1917-1921; Labow et al. (1990) Mol. Cell. Biol. 10:3343-3356; Zambretti et al. (1992) PNAS 89:3952-3956; Balm et al. (1991) PNAS 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) PNAS 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724; Bourouis et al., EMBO J. 2(7): 1099-1104 (1983) White et al., Nucl Acids Res 18: 1062 (1990), Spencer et al., Theon Appl Genet 79: 625-631(1990), U.S. Pat. Nos. 5,034,322; 6,174,724; 6,255,560; 4,795,855; 5,378,824; and 6,107,549. Such disclosures are herein incorporated by reference.

The above list of selectable marker genes is not limiting. Any selectable marker gene can be used in the present invention. Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) PNAS 90:11212-11216; Christou, P. (1993) In Vitro Cell. Dev. Biol.-Plant; 29P:119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. A leader, for example, is operably linked to coding sequence when it is capable of serving as a leader for the polypeptide encoded by the coding sequence.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present invention. See, for example, U.S. Pat. No. 7,250,554, which utilizes P-DNA instead of T-DNA in the transformation vector. Agrobacterium tumefaciens is a naturally occurring bacterium that is capable of inserting its DNA (genetic information) into plants, resulting in a type of injury to the plant known as crown gall. A transgenic plant formed using Agrobacterium transformation methods typically contains a single gene on one chromosome, although multiple copies are possible. Such transgenic plants can be referred to as being hemizygous for the added gene.

Other methods utilized for the delivery foreign DNA or other foreign nucleic acids involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet. 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988); Methods in Plant Molecular Biology (Schuler and Zielinski, eds.) Academic Press, Inc. (1989); M. E. Fromm et al., Nature, 319, 791 (1986); H. Jones et al., Plant Mol. Biol., 13, 501 (1989) and H. Yang et al., Plant Cell Reports, 7, 421 (1988); UMizuno et al., 2004; Petolino et al., 2000; U.S. Pat. No. 5,302,523; and US Application Publication No. 20040197909; Kaepler et al., 1992; Raloff, 1990; Wang, 1995; U.S. Pat. Nos. 5,204,253; 5,015,580; 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and Raineri et al., Bio/Tech. 8:33-38 (1990), each of which is incorporated herein by reference in its entirety). The method of transformation depends upon the plant cell to be transformed, stability of vectors used, expression level of gene products and other parameters. Specific methods for transforming certain plant species (e.g., maize, rice, wheat, barley, soybean) are described in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830, each of which is incorporated by reference in its entirety.

Other suitable methods of introducing nucleotide sequences into plant cells and subsequent insertion into the plant genome include microinjection as Crossway et al. (1986) Biotechniques 4:320-334, electroporation as described by Riggs et al. (1986) PNAS 83:5602-5606, Agrobacterium-mediated transformation as described by Townsend et al., U.S. Pat. No. 5,563,055, Zhao et al., U.S. Pat. No. 5,981,840, Yukou et al., WO 94/000977, and Hideaki et al., WO 95/06722, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) PNAS 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) PNAS 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

In some embodiments, the polynucleotides of the invention may be introduced into plants using a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as resistance to an insect pest, and to introduce these genes into already useful varieties of plants. Once the foreign genes have been introduced into a plant, that plant can then be used in conventional plant breeding schemes (e.g., pedigree breeding, single-seed-descent breeding schemes, reciprocal recurrent selection) to produce progeny which also contain the gene of interest.

In some embodiments, genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

In this manner, the present invention provides transformed seed (also referred to as “transgenic seed”) having a polynucleotide construct of the invention, for example, an expression cassette of the invention, stably incorporated into their genome.

In specific embodiments, the nucleic acid molecules and polynucleotide constructs of the present invention can be provided to a plant using a variety of transient transformation methods. Such transient transformation methods include, but are not limited to, the introduction of the sequence or variants and fragments thereof directly into the plant or the introduction of a transcript into the plant. Such methods include, for example, microinjection, electroporation, or particle bombardment. See, for example, Crossway et al. (1986) Mol Gen. Genet. 202:179-185; Nomura et al. (1986) Plant Sci. 44:53-58; Hepler et al. (1994) PNAS 91: 2176-2180 and Hush et al. (1994) The Journal of Cell Science 107:775-784, Sheen, J. 2002. A transient expression assay using maize mesophyll protoplasts. Sheen, J. 2001. Signal transduction in maize and Arabidopsis mesophyll protoplasts. Plant Physiol. 2001 December; 127:1466-1475, Anderson et al., U.S. Pat. No. 7,645,919 B2, all of which are herein incorporated by reference. Alternatively, the polynucleotide can be transiently transformed into the plant using techniques known in the art.

The constructs of the present invention may be provided, in one embodiment, as double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present invention.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see, for example, Molecular Cloning: A Laboratory Manual, 3^{rd} edition Volumes 1, 2, and 3 (2000) J. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, FL. (1993)). Typical vectors useful for expression of nucleic acids in higher plants are well known in the art and include vectors derived from the tumor-inducing (Ti) plasmid of Agrobacterium tumefaciens (Rogers, et al., Methods in Enzymology 153: 253-277 (1987)). Other recombinant vectors useful for plant transformation, including the pCaMVCN transfer control vector, have also been described in the scientific literature (see, for example, Fromm, et al., Proc. Natl. Acad. Sci. USA 82: 5824-5828 (1985)).

Various regulatory elements may be included in a construct including any of those provided herein. Any such regulatory elements may be provided in combination with other regulatory elements, e.g. any combination of sequences presented as SEQ ID NO: 1-24. Such combinations can be designed or modified to produce desirable regulatory features. In one embodiment, constructs of the present invention comprise at least one regulatory element operably linked to at least one transcribable polynucleotide molecule operably linked to at least one 3′ UTR.

Constructs of the present invention may include any promoter or leader provided herein or known in the art. For example, a promoter of the present invention may be operably linked to a heterologous non-translated 5′ leader such as one derived from a heat shock protein gene (see, for example, U.S. Pat. Nos. 5,659,122 and 5,362,865). Alternatively, a leader of the present invention may be operably linked to a heterologous promoter such as the Cauliflower Mosaic Virus 35S transcript promoter (see, U.S. Pat. No. 5,352,605).

As used herein, the term “intron” refers to a DNA molecule that may be isolated or identified from the genomic copy of a gene and may be defined generally as a region spliced out during mRNA processing prior to translation. Alternately, an intron may be a synthetically produced or manipulated DNA element. An intron may contain enhancer elements that effect the transcription of operably linked genes. An intron may be used as a regulatory element for modulating expression of an operably linked transcribable polynucleotide molecule. A DNA construct may comprise an intron, and the intron may or may not be heterologous with respect to the transcribable polynucleotide molecule sequence. Examples of introns in the art include the rice actin intron (U.S. Pat. No. 5,641,876) and the corn HSP70 intron (U.S. Pat. No. 5,859,347). Further, when modifying intron/exon boundary sequences, it may be preferable to avoid using the nucleotide sequence AT or the nucleotide A just prior to the 5′ end of the splice site (GT) and the nucleotide G or the nucleotide sequence TG, respectively just after 3′ end of the splice site (AG) to eliminate the potential of unwanted start codons from being formed during processing of the messenger RNA into the final transcript. The sequence around the 5′ or 3′ end splice junction sites of the intron can thus be modified in this manner.

As used herein, the term “3′ transcription termination molecule” or “3′ UTR” refers to a DNA molecule that is used during transcription to produce the 3′ untranslated region (3′ UTR) of an mRNA molecule. The 3′ untranslated region of an mRNA molecule may be generated by specific cleavage and 3′ polyadenylation, a.k.a. polyA tail. A 3′ UTR may be operably linked to and located downstream of a transcribable polynucleotide molecule and may include polynucleotides that provide a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region (see, Fraley, et al., Proc. Natl. Acad. Sci. USA, 80: 4803-4807 (1983)); wheat hsp17 3′ region; pea rubisco small subunit 3′ region; cotton E6 3′ region (U.S. Pat. No. 6,096,950); 3′ regions disclosed in WO0011200A2; and the coixin 3′ UTR (U.S. Pat. No. 6,635,806).

### Transcribable Polynucleotide Molecules

As used herein, the term “transcribable polynucleotide molecule” refers to any DNA molecule capable of being transcribed into a RNA molecule, including, but not limited to, those having protein coding sequences and those producing RNA molecules having sequences useful for gene suppression. The type of DNA molecule can include, but is not limited to, a DNA molecule from the same plant, a DNA molecule from another plant, a DNA molecule from a different organism, or a synthetic DNA molecule, such as a DNA molecule containing an antisense message of a gene, or a DNA molecule encoding an artificial, synthetic, or otherwise modified version of a transgene. Exemplary transcribable DNA molecules for incorporation into constructs of the invention can include, e.g., DNA molecules or genes from a species other than the species into which the DNA molecule is incorporated or genes that originate from, or are present in, the same species, but are incorporated into recipient cells by genetic engineering methods rather than classical breeding techniques.

A “transgene” refers to a transcribable polynucleotide molecule heterologous to a host cell at least with respect to its location in the genome and/or a transcribable polynucleotide molecule artificially incorporated into a host cell's genome in the current or any prior generation of the cell.

A promoter of the present invention, such as SEQ ID NOs: 1-24, may be operably linked to a transcribable polynucleotide molecule that is heterologous with respect to the promoter molecule. As used herein, the term “heterologous” refers to the combination of two or more polynucleotide molecules when such a combination is not normally found in nature. For example, the two molecules may be derived from different species and/or the two molecules may be derived from different genes, e.g. different genes from the same species or the same genes from different species. Additionally, the two molecules may be derived from isolated locations in the same gene, wherein such a combination of molecules is not normally found in nature. A promoter is thus heterologous with respect to an operably linked transcribable polynucleotide molecule if such a combination is not normally found in nature, i.e. that transcribable polynucleotide molecule is not naturally occurring operably linked in combination with that promoter molecule.

As used herein, the term “overexpression” refers to an increased expression level of a transcribable polynucleotide molecule or a protein in a plant, plant cell or plant tissue, compared to expression in a wild-type plant, cell or tissue, at any developmental or temporal stage for the gene. Overexpression can take place in plant cells normally lacking expression of a transcribable polynucleotide molecule of interest. Overexpression can also occur in plant cells where endogenous expression of a transcribable polynucleotide molecule or functionally equivalent molecules normally occurs, but such endogenous expression is at a lower level compared to the overexpression. Overexpression thus results in a greater than endogenous production, or “overproduction” of the polypeptide in the plant, cell or tissue.

In certain embodiments, the expression or overexpression of a transcribable polynucleotide molecule as disclosed herein can effect an enhanced trait or altered phenotype directly or indirectly. In some cases it may do so, for example, by expressing one or more genes with spatio-temporal precision to effectively increase yield. In certain exemplary embodiments, the protein produced from the transcribable polynucleotide molecule can lead to increased starch content in a plant.

The transcribable polynucleotide molecule may generally be any DNA molecule for which expression of a RNA transcript is desired. Such expression of an RNA transcript may result in translation of the resulting mRNA molecule and thus protein expression. Alternatively, for example, a transcribable polynucleotide molecule may be designed to ultimately cause decreased expression of a specific gene or protein. In one embodiment, this may be accomplished by using a transcribable polynucleotide molecule that is oriented in the antisense direction. One of ordinary skill in the art is familiar with using such antisense technology. Briefly, as the antisense transcribable polynucleotide molecule is transcribed, the RNA product hybridizes to and sequesters a complementary RNA molecule inside the cell. This duplex RNA molecule cannot be translated into a protein by the cell's translational machinery and is degraded in the cell. Any gene may be negatively regulated in this manner.

Thus, one embodiment of the invention is a regulatory element of the present invention, such as those provided as SEQ ID NOs: 1-24, operably linked to a transcribable polynucleotide molecule so as to modulate transcription of the transcribable polynucleotide molecule at a desired level or in a desired pattern when the construct is integrated in the genome of a plant cell. In one embodiment, the transcribable polynucleotide molecule comprises a protein-coding region of a gene, and the promoter affects the transcription of an RNA molecule that is translated and expressed as a protein product.

In another embodiment, the transcribable polynucleotide molecule comprises an antisense region of a gene, and the promoter affects the transcription of an antisense RNA molecule, double stranded RNA or other similar inhibitory RNA molecule in order to inhibit expression of a specific RNA molecule of interest in a target host cell.

### Genes of Agronomic Interest

Transcribable polynucleotide molecules may be genes of agronomic interest. As used herein, the term “gene of agronomic interest” refers to a transcribable polynucleotide molecule that when expressed in a particular plant tissue, cell, or cell type confers a desirable characteristic, such as associated with plant morphology, physiology, growth, development, yield, grain composition, product, nutritional profile, disease or pest resistance, environmental or chemical tolerance, and/or may act as a pesticidal agent in the diet of a pest that feeds on the plant. In one embodiment of the invention, a regulatory element of the invention is incorporated into a construct such that the regulatory element is operably linked to a transcribable DNA molecule that is a gene of agronomic interest.

Genes of agronomic interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly.

In a transgenic plant of the present invention, the expression of the gene of agronomic interest can confer a beneficial agronomic trait. A beneficial agronomic trait may include, for example, but is not limited to genes encoding important or desired traits for agronomics, herbicide tolerance or resistance, insect control, insect resistance, modified yield, disease resistance, pathogen resistance, modified plant growth and development, modified starch content, grain characteristics, modified oil content, modified fatty acid content, modified protein content, modified fruit ripening, abiotic stress tolerance, enhanced animal and human nutrition, biopolymer productions, environmental stress resistance, pharmaceutical peptides, sterility, improved processing qualities, improved flavor, hybrid seed production utility, improved fiber production, commercial product production, and biofuel production.

Examples of genes of agronomic interest can include, but are not limited to, genes encoding proteins important for agronomics, such as a yield protein, a stress resistance protein, a developmental control protein, a tissue differentiation protein, a meristem protein, an environmentally responsive protein, a senescence protein, a hormone responsive protein, an insect resistant protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. The product of a gene of agronomic interest may act within the plant in order to cause an effect upon the plant physiology or metabolism.

In certain embodiments, promoter sequences disclosed herein as SEQ ID NOs: 1-24 are useful for the expression of transgenes improving photosynthesis and sucrose production, sucrose export, drought resistance, and pest resistance. For example, the promoters of pMePsbr (SEQ ID NO: 3) or pAtCAB1 (SEQ ID NO: 6), and other promoters specific for autotrophic tissues, are particularly useful for driving expression of these genes. Examples of genes of agronomic interest involved in improving photosynthesis and yield may alter non-photochemical quenching (NPQ), reduce photorespiration, or improve Calvin Cycle efficiency, and include, but are not limited to, Violaxanthin de-epoxidase (VDE), Zeaaxanthine epoxidase (ZEP), and Photosystem II Subunit S (PsbS) which alter plant NPQ responses (Kromdijk et al. (2016)); as well as Glycolate dehydrogenase, Malate Synthase, and knockdown of Glycolate/Glycerate transporter genes which introduce a photorespiratory bypass (South et al. (2019); as well as cyanobacterial bi-functional enzyme Fructose-1,6-bisphosphate/Seduheptulose-1,7-Bisphophate or Seduheptulose-1,7-bisphosphate, together with red algal gene Cytochrome C6 (Lopez-Calcagno et al. (2020). Examples of genes of agronomic interest involved in improving sucrose production and yield by altering sugar metabolism include E. coli Pyrophosphatase (PPase) or sequences for RNAi targeting leaf ADP-Glucose Pyrophosphorylase (AGPase) improving sucrose production and export (Jonik et al. (2012). Examples of genes of agronomic interest involved in drought resistance include brassinosteroid receptor family gene BRL3 to improve drought resistance without negatively impacting yield as demonstrated by (Fabregas et al., 2018). Examples of genes of agronomic interest involved in pest resistance include, but are not limited to DNA Polymerase Δ Subunit 1 (MePOLD1), a gene that has been demonstrated to mediate resistance to ACMV by Lim et al. (2022).

In certain embodiments, promoter sequences disclosed herein as SEQ ID NOs: 1-24 are useful for the expression of transgenes improving sucrose and nitrogen allocation within cassava. For example, the promoters of pMeSUS1 (SEQ ID NO: 5) and pMeSWEET1-like (SEQ ID NO: 1), and other promoters specific for phloem/phloem parenchyma, are particularly useful for driving expression of genes of agronomic interest involved in improving expression of sugar transporters and amino acid transporters supporting either phloem loading or phloem unloading of sugars and amino acids. Examples of genes of agronomic interest involved in expression of sugar transporters and amino acid transporters include, but are not limited to Sucrose Will Eventually Be Exported (SWEET)-family proteins, Sucrose Transporter (SUT/SUC)-family proteins, UMAMIT-family proteins, Amino Acid Permease (AAP)-family proteins. Examples of genes of agronomic interest useful in improving sink strength by improving sucrose consumption include, but are not limited to, Sucrose Synthase (SUS)-family proteins. Examples of genes of agronomic interest able to block sugar transporters, altering the sugar distribution within the plant, include, but are not limited to Flowering Locus T (FT)-related genes like Self-Pruning 6A (SP6A) genes, which have been demonstrated to be able to block SWEET transport proteins (Abelenda et al. (2019).

In certain embodiments, promoter sequences disclosed herein as SEQ ID NOs: 1-24 are useful for the expression of transgenes improving starch yield, storage root size, starch quality, nutritional quality, pest resistance, or post-harvest physiological deterioration. For example, the promoter of pMeGPT (SEQ ID NO: 2) and other promoters specific for storage root) are particularly useful for driving expression of genes of agronomic interest involved in improving starch yield, storage root size, starch quality, nutritional quality, pest resistance, or post-harvest physiological deterioration. Examples of genes of agronomic interest involved in altering sugar- and starch metabolism in the storage root include, but are not limited to genes altering cytosolic sugar conversion enzymes like Sucrose Synthases (SUS) genes, Fructokinases (FRK) genes, UDP-Glucose-Pyrophosphorylase (UGP) genes, Phosphoglucoisomerases (PGI) genes, and Phosphoglucomutases (PGM) genes. Examples of genes of agronomic interest involved in the import of starch precursor metabolites include, but are not limited to, Glucose-6-Phosphate Phosphate Translocators (GPTs) or Nucleotide Transporter family proteins, which have been demonstrated to improve starch concentration by Zhang et al. (2008) and Jonik et al. (2012). Examples of genes of agronomic interest involved in amyloplastic starch formation include, but are not limited to, Phosphoglucomutase genes, ADP-Glucose Pyrophosphorylase (AGPase) genes, Soluble Starch Synthase genes, Granule-Bound Starch Synthase genes. Examples of genes of agronomic interest involved in starch granule targeting, starch granule remodeling or starch breakdown include, but are not limited to, Alpha- and Beta-Amylase genes, Debranching Enzyme genes (DBE), Starch Phosphorylation genes, Early Starvation (ESV) genes, Protein Targeting to Starch (PTST) genes, Myosin-Resembling Chloroplast Protein (MRC) genes, or Mar Binding Filament-Like Protein 1 (MFP1). Examples of genes of agronomic interest involved in altering hormonal regulation include, but are not limited to, cytokinine synthesis genes like Isopentenyl Transferase Genes (IPT), CYP735A genes, or Lonely Guy (LOG) genes. Examples of genes of agronomic interest altering auxin synthesis genes include, but are not limited to, YUCCA genes. Genes of agronomic interest involved in the formation of vitamins or in the acquisition of minerals include, but are not limited to, genes involved in ß-carotene synthesis such as Pytoene Synthase (PTS) genes (Welsch et al. (2010), or genes involved in vitamin b6 synthesis such as PDX1 and PDX2 (Li et al. (2015)), or genes involved in mineral accumulation such as Vacuolar Iron Transporter (VIT) genes, Iron Transporter (IRT) genes, and Ferritin (FER) genes (Narayanan et al. (2019)). Promoter sequences as described herein may further be used to drive genes involved in root pathogen resistance or genes decreasing post-harvest deterioration. Examples of genes for decreasing post-harvest deterioration include, but are not limited to, redox-protective genes like Peroxidase (PER) genes, Glutathione Reductase (GSR) genes, or Ascorbate Peroxidase (APX) genes (Vanderschuren et al. (2014)).

In certain embodiments, promoter sequences disclosed herein as SEQ ID NOs: 1-24 are useful for the expression of transgenes improving storage root size by altering vascular cambium activity. For example, the promoter of pManes.14g071100 (SEQ ID NO: 4), and other promoters specific for storage root cambium, are useful for the expression of transgenes improving storage root size by altering vascular cambium activity. Examples of genes of agronomic interest involved in altering hormonal activity or genes altering vascular cambium cell division or cell differentiation include, but are not limited to ANTINTEGUMENTA genes or genes involved in xylem cell formation like genes of the WUSCHEL-RELATED-HOMEOBOX gene family, MONOPTEROUS genes, and BREDIPEDICELLUS/KNAT genes. Auxin-biosynthesis genes like YUCCA, AUXIN RESPONSE FACTOR family genes, cytokinine-biosynthesis genes like LONELY GUY (LOG) genes, and cytokinine-responsive genes like LATERAL BORDER DOMAIN (LBDs) genes are other examples of genes of agronomic interest useful with the promoter sequences disclosed herein.

Alternatively, a gene of agronomic interest can affect the above mentioned plant characteristic or phenotype by encoding an RNA molecule that causes the targeted modulation of gene expression of an endogenous gene, for example via antisense (see e.g. U.S. Pat. No. 5,107,065); inhibitory RNA (“RNAi”, including modulation of gene expression via miRNA-, siRNA-, trans-acting siRNA-, and phased sRNA-mediated mechanisms, e.g. as described in published applications US 2006/0200878 and US 2008/0066206, and in U.S. patent application Ser. No. 11/974,469); or cosuppression-mediated mechanisms. The RNA could also be a catalytic RNA molecule (e.g. a ribozyme or a riboswitch; see e.g. US 2006/0200878) engineered to cleave a desired endogenous mRNA product. Thus, any transcribable polynucleotide molecule that encodes a transcribed RNA molecule that affects an agronomically important phenotype or morphology change of interest may be useful for the practice of the present invention. Methods are known in the art for constructing and introducing constructs into a cell in such a manner that the transcribable polynucleotide molecule is transcribed into a molecule that is capable of causing gene suppression. For example, posttranscriptional gene suppression using a construct with an anti-sense oriented transcribable polynucleotide molecule to regulate gene expression in plant cells is disclosed in U.S. Pat. Nos. 5,107,065 and 5,759,829, and posttranscriptional gene suppression using a construct with a sense-oriented transcribable polynucleotide molecule to regulate gene expression in plants is disclosed in U.S. Pat. Nos. 5,283,184 and 5,231,020. Expression of a transcribable polynucleotide in a plant cell can also be used to suppress plant pests feeding on the plant cell, for example, compositions isolated from coleopteran pests (U.S. Patent Publication No. US20070124836) and compositions isolated from nematode pests (U.S. Patent Publication No. US20070250947). Plant pests include, but are not limited to arthropod pests, nematode pests, and fungal or microbial pests. Exemplary transcribable polynucleotide molecules for incorporation into constructs of the present invention include, for example, DNA molecules or genes from a species other than the target species or genes that originate with or are present in the same species, but are incorporated into recipient cells by genetic engineering methods rather than classical reproduction or breeding techniques. The type of polynucleotide molecule can include, but is not limited to, a polynucleotide molecule that is already present in the plant cell, a polynucleotide molecule from another plant, a polynucleotide molecule from a different organism, or a polynucleotide molecule generated externally, such as a polynucleotide molecule containing an antisense message of a gene, or a polynucleotide molecule encoding an artificial, synthetic, or otherwise modified version of a transgene.

Genes of interest can include, for example, those genes involved in information, such as zinc fingers, those involved in communication, such as kinases, and those involved in housekeeping, such as heat shock proteins; those involved in oil, starch, carbohydrate, or nutrient metabolism; genes encoding enzymes and other proteins from plants and other sources including prokaryotes and other eukaryotes.

Examples of genes of agronomic interest known in the art include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. RE37,543; 6,228,623; and U.S. Pat. Nos. 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

Alternatively, a gene of agronomic interest can affect the above mentioned plant characteristics or phenotypes by encoding an RNA molecule that causes the targeted modulation of gene expression of an endogenous gene, for example by antisense (see, e.g., U.S. Pat. No. 5,107,065); inhibitory RNA (“RNAi,” including modulation of gene expression by miRNA-, siRNA-, trans-acting siRNA-, and phased sRNA-mediated mechanisms, e.g., as described in published applications U.S. 2006/0200878 and U.S. 2008/0066206, and in U.S. patent application Ser. No. 11/974,469); or cosuppression-mediated mechanisms. The RNA could also be a catalytic RNA molecule (e.g., a ribozyme or a riboswitch; see, e.g., U.S. 2006/0200878) engineered to cleave a desired endogenous mRNA product. Methods are known in the art for constructing and introducing constructs into a cell in such a manner that the transcribable DNA molecule is transcribed into a molecule that is capable of causing gene suppression.

### Selectable Markers

Selectable marker transgenes may also be used with the regulatory elements of the invention. As used herein the term “selectable marker transgene” refers to any transcribable DNA molecule whose expression in a transgenic plant, tissue or cell, or lack thereof, can be screened for or scored in some way. As used herein the term “marker” refers to any transcribable polynucleotide molecule whose expression, or lack thereof, can be screened for or scored in some way. Marker genes for use in the practice of the present invention include, but are not limited to, transcribable polynucleotide molecules encoding ß-glucuronidase (GUS described in U.S. Pat. No. 5,599,670), green fluorescent protein and variants thereof (GFP described in U.S. Pat. Nos. 5,491,084 and 6,146,826), proteins that confer antibiotic resistance, or proteins that confer herbicide tolerance.

Included within the term “selectable markers” are also genes which encode a secretable marker whose secretion can be detected as a means of identifying or selecting for transformed cells. Examples include markers that encode a secretable antigen that can be identified by antibody interaction, or even secretable enzymes which can be detected catalytically. Selectable secreted marker proteins fall into a number of classes, including small, diffusible proteins which are detectable, (e.g. by ELISA), small active enzymes which are detectable in extracellular solution (e.g., alpha-amylase, beta-lactamase, phosphinothricin transferase), or proteins which are inserted or trapped in the cell wall (such as proteins which include a leader sequence such as that found in the expression unit of extension or tobacco pathogenesis related proteins also known as tobacco PR-S). Other possible selectable marker genes will be apparent to those of skill in the art and are encompassed by the present invention.

“Selectable markers” refer to sequences that can be used to distinguish between transformed and non-transformed genes. Reporter genes are test sequences whose expression can be quantified. Reporter genes can act as markers for transformed genes. In some embodiments, the transgenes of the present invention comprise at least one reporter gene. As used herein a “reporter” or a “reporter gene” refers to a nucleic acid molecule encoding a detectable marker. The reporter gene can be, for example, luciferase (e.g., firefly luciferase or Renilla luciferase), β-galactosidase, chloramphenicol acetyl transferase (CAT), or a fluorescent protein (e.g., green fluorescent protein (GFP), red fluorescent protein (DsRed), yellow fluorescent protein, blue fluorescent protein, cyan fluorescent protein, or variants thereof, including enhanced variants such as enhanced GFP (eGFP). Reporter genes are detectable by a reporter assay. Reporter assays can measure the level of reporter gene expression or activity by any number of means, including, for example, measuring the level of reporter mRNA, the level of reporter protein, or the amount of reporter protein activity. Reporter assays are known in the art or otherwise disclosed herein.

### Cell Transformation

The invention is also directed to a method of producing transformed cells and plants which comprise a promoter operably linked to a transcribable polynucleotide molecule.

The term “transformation” refers to the introduction of nucleic acid into a recipient host. As used herein, the term “host” refers to bacteria, fungi, or plant, including any cells, tissue, organs, or progeny of the bacteria, fungi, or plant. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign polynucleotide molecule, such as a construct, has been introduced. The introduced polynucleotide molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic organism as a parent in a cross and exhibiting an altered phenotype resulting from the presence of a foreign polynucleotide molecule. The term “transgenic” refers to a bacteria, fungi, or plant containing one or more heterologous polynucleic acid molecules. The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source or species, is modified from its original form and/or genetic locus; is heterologous to a host cell at least with respect to its location in the genome; the promoter is not the native promoter for the operably linked polynucleotide, and/or is artificially incorporated into a host cell's genome in the current or any prior generation of the cell.

There are many methods for introducing polynucleic acid molecules into plant cells. The method generally comprises the steps of selecting a suitable host cell, transforming the host cell with a recombinant vector, and obtaining the transformed host cell. Suitable methods include bacterial infection (e.g. Agrobacterium), binary bacterial artificial chromosome vectors, direct delivery of DNA (e.g. via PEG-mediated transformation, desiccation/inhibition-mediated DNA uptake, electroporation, agitation with silicon carbide fibers, and acceleration of DNA coated particles, etc. (reviewed in Potrykus, et al., Ann. Rev. Plant Physiol. Plant Mol. Biol. 42: 205 (1991)), gene editing (e.g., CRISPR-Cas systems), among others.

Technology for introduction of a DNA molecule into cells is well known to those of skill in the art. Methods and materials for transforming plant cells by introducing a plant DNA construct into a plant genome in the practice of this invention can include any of the well-known and demonstrated methods. Any transformation methods may be utilized to transform a host cell with one or more promoters and/or constructs of the present. Host cells may be any cell or organism such as a plant cell, algae cell, algae, fungal cell, fungi, bacterial cell, or insect cell. Preferred hosts and transformed cells include cells from: plants, Aspergillus, yeasts, insects, bacteria and algae. In specific embodiments, the host cells and transformed cells may include cells from crop plants.

In various embodiments, the methods described herein can involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

“Stable transformation” is intended that the polynucleotide construct introduced into a plant integrates into the genome of the plant and is capable of being inherited by progeny thereof. “Transient transformation” is intended that a polynucleotide construct introduced into a plant does not integrate into the genome of the plant.

A transgenic plant subsequently may be regenerated from a transgenic plant cell of the invention. Using conventional breeding techniques or self-pollination, seed may be produced from this transgenic plant. Such seed, and the resulting progeny plant grown from such seed, will contain the recombinant DNA molecule of the invention, and therefore will be transgenic.

Regenerated transgenic plants can be self-pollinated to provide homozygous transgenic plants (homozygous for the recombinant DNA molecule). Alternatively, pollen obtained from the regenerated transgenic plants may be crossed with non-transgenic plants to provide seed for heterozygous transgenic plants (heterozygous for the recombinant DNA molecule), preferably inbred lines of agronomically important species. Both such homozygous and heterozygous transgenic plants are referred to herein as “progeny plants.” Progeny plants are transgenic plants descended from the original transgenic plant and containing the recombinant DNA molecule of the invention. Seeds produced using a transgenic plant of the invention can be harvested and used to grow generations of transgenic plants, i.e., progeny plants of the invention, comprising the construct of this invention and expressing a gene of agronomic interest. Descriptions of breeding methods that are commonly used for different traits and crops can be found in one of several reference books, see, for example, Allard, Principles of Plant Breeding, John Wiley & Sons, NY, U. of CA, Davis, CA, 50-98 (1960); Simmonds, Principles of crop improvement, Longman, Inc., NY, 369-399 (1979); Sneep and Hendriksen, Plant breeding perspectives, Wageningen (ed), Center for Agricultural Publishing and Documentation (1979); Fehr, Soybeans: Improvement, Production and Uses, 2nd Edition, Monograph, 16:249 (1987); Fehr, Principles of variety development, Theory and Technique, (Vol. 1) and Crop Species Soybean (Vol 2), Iowa State Univ., Macmillan Pub. Co., NY, 360-376 (1987). Conversely, pollen from non-transgenic plants may be used to pollinate the regenerated transgenic plants.

The transformed plants may be analyzed for the presence of the genes of interest and the expression level and/or profile conferred by the regulatory elements of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays. The expression of a transcribable polynucleotide molecule can be measured using TaqMan® (Applied Biosystems, Foster City, CA) reagents and methods as described by the manufacturer and PCR cycle times determined using the TaqMan® Testing Matrix. Alternatively, the Invader® (Third Wave Technologies, Madison, WI) reagents and methods as described by the manufacturer can be used to evaluate transgene expression.

The seeds of the plants of this invention can be harvested from fertile transgenic plants and be used to grow progeny generations of transformed plants of this invention including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. Plant parts of the invention may be viable, nonviable, regenerable, and/or non-regenerable. The invention also includes and provides transformed plant cells comprising a DNA molecule of the invention. The transformed or transgenic plant cells of the invention include regenerable and/or non-regenerable plant cells. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immuno-precipitation, and ELISA.

### Commodity Products

The present invention provides a commodity product comprising DNA molecules according to the invention. As used herein, a “commodity product” refers to any composition or product which is comprised of material derived from a plant, seed, plant cell or plant part comprising a DNA molecule of the invention. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the invention can thus be used to manufacture any commodity product typically acquired from plants or parts thereof. A commodity product of the invention will contain a detectable amount of DNA corresponding to the recombinant DNA molecule of the invention. Detection of one or more of this DNA in a sample may be used for determining the content or the source of the commodity product. Any standard method of detection for DNA molecules may be used, including methods of detection disclosed herein.

### Plants

The nucleic acid molecules and polynucleotide constructs of the present invention can be used for transformation of any plant species, including, but not limited to, monocots and dicots. Examples of plant species of interest include, but are not limited to, Arabidopsis thaliana, peppers (Capsicum spp; e.g., Capsicum annuum, C. baccatum, C. chinense, C. frutescens, C. pubescens, and the like), cowpea (Vigna unguiculata), tomatoes (Lycopersicon esculentum), tobacco (Nicotiana tabacum), eggplant (Solanum melongena), petunia (Petunia spp., e.g., Petunia x hybrida or Petunia hybrida), corn or maize (Zea mays), Brassica ssp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), green millet (Setaria viridis), finger millet (Eleusine coracana)), sunflower (Helianthus annuus), safflower (Carthamus tinctorius), wheat (Triticum aestivum), soybean (Glycine max), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), switchgrass (Panicum virgatum), algae (e.g., Chlamydomonas reinhardtii, Botryococcus braunii, Chlorella spp., Dunaliella tertiolecta, Gracilaria spp.), oats, barley, vegetables, ornamentals, and conifers. The nucleic acid molecules and polynucleotide constructs of the present invention can also be used for transformation of any algae species.

As used herein, the term “plant” refers to any living organism belonging to the kingdom Plantae (i.e., any genus/species in the Plant Kingdom). In some embodiments, the plant is a tree, herb, bush, grass, vine, fern, moss or green algae. The plant may be monocotyledonous (monocot), or dicotyledonous (dicot). Examples of particular plants that may comprise a polynucleotide of the invention include but are not limited to Arabidopsis, Brachypodium, switchgrass, corn, potato, rose, apple tree, sunflower, wheat, rice, bananas, plantains, tomatoes, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, cauliflower, cavalo, collards, cowpea, kale, kohlrabi, mustard greens, rape greens, and other brassica leafy vegetable crops, bulb vegetables (e.g., garlic, leek, onion (dry bulb, green, and Welch), shallot), citrus fruits (e.g., grapefruit, lemon, lime, orange, tangerine, citrus hybrids, pummelo), cucurbit vegetables (e.g., cucumber, citron melon, edible gourds, gherkin, muskmelons (including hybrids and/or cultivars of Cucumis melons), water-melon, cantaloupe, and other cucurbit vegetable crops), fruiting vegetables (including eggplant, ground cherry, pepino, pepper, tomato, tomatillo), grape, leafy vegetables (e.g., romaine), root/tuber and corm vegetables (e.g., potato, yam, cassava, taro), tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, blackberries, cloudberries, loganberries, raspberries, salmonberries, thimbleberries, and wineberries), cereal crops (e.g., corn (maize), rice, wheat, barley, sorghum, millets, oats, ryes, triticales, buckwheats, fonio, quinoa, oil palm), Brassicaceae family plants, and Fabaceae family plants, pome fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), fiber crops (e.g., hemp, cotton), ornamentals, and the like.

Having now generally described the invention, the same will be more readily understood through reference to the following examples which are provided by way of illustration, and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1: Activity and Tissue Specificity of Various Promoters in Transgenic, Field-Grown Cassava Plants

Transgenic cassava plants expressing various combinations of metabolically active genes, altering photosynthetic-, transport-, and storage metabolism, were field-tested at NCHU experimental station in Taichung, Taiwan. Cassava plants (genotype 60444) were transformed as described previously (Bull et al., 2009). Plants were transformed with one of seven different vectors, each vector comprising three to six candidate promoters each driving a different detectable coding sequence. A total of ten candidate promoter elements were tested to determine their expression profiles. The promoters were evaluated based on specificity for autotrophic (i.e. “source” tissues) and/or heterotrophic (i.e. “sink” tissues).

TABLE 1
Summary of ten promoters tested in field-grown cassava plants

| | | | | | Results |
|---|---|---|---|---|---|
| SEQ ID | | | Gene/NCBI | Analyzed | shown |
| NO: | Promoter | Code | identifier | by | in FIG. |
| 6 | CHLOROPHYLL | AtCAB1 | At1g29930 | Histology | 1 & 2 |
| | A/B-BINDING | | | and qPCR | |
| | PROTEIN | | | | |
| 10 | FRUCTOSE- | AtFBA2 | AT4G38970 | qPCR | 1 |
| | BISPHOSPHATE | | | | |
| | ALDOLASE 2 | | | | |
| 12 | GLYCERALDEHYDE | AtGAPA | AT3G26650 | qPCR | 1 |
| | 3-PHOSPHATE | | | | |
| | DEHYDROGENASE | | | | |
| | SUBUNIT A | | | | |
| 14 | GRANULE-BOUND | MeGBSS1 | Manes.02G001000 | Histology | 1 & 2 |
| | STARCH | | | and qPCR | |
| | SYNTHASE1 | | | | |
| 15 | LEAF-SPECIFIC 1 | StLS1 | X04753.1 | Histology | 1 & 2 |
| | | | | and qPCR | |
| 18 | RIBULOSE | AtRBCS1A | AT1G67090 | qPCR | 1 |
| | BISPHOSPHATE | | | | |
| | CARBOXYLASE | | | | |
| | SMALL SUBUNIT 1A | | | | |
| 19 | RIBULOSE | SlRBCS2 | X66069.1 | qPCR | 1 |
| | BISPHOSPHATE | | | | |
| | CARBOXYLASE | | | | |
| | SMALL SUBUNIT 2 | | | | |
| 20 | RIBULOSE | AtRBCS3B | At5g38410 | Histology | 1 & 2 |
| | BISPHOSPHATE | | | and qPCR | |
| | CARBOXYLASE | | | | |
| | SMALL SUBUNIT 3 | | | | |
| 21 | SOLUBLE STARCH | StSSS3 | X95759.1 | Histology | 1 & 2 |
| | SYNTHASE 3 | | | and qPCR | |
| 22 | STARCH | StSTP1 | X73684.1 | Histology | 1 & 2 |
| | PHOSPHORYLASE 1 | | | and qPCR | |

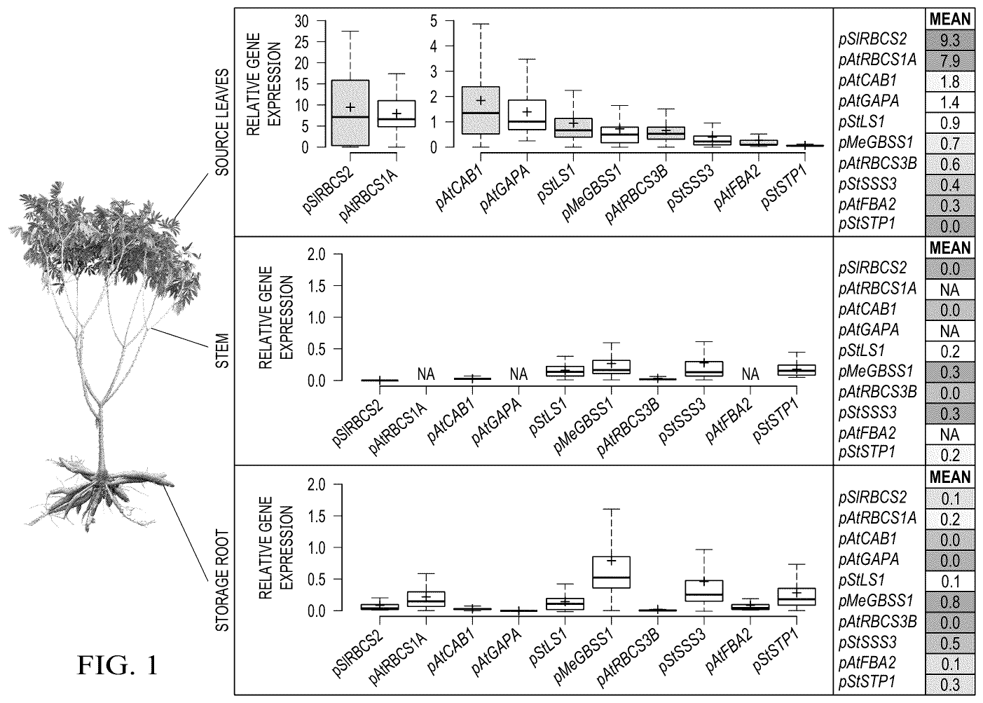

Over 400 field-grown plants, generated using 7 different constructs and representing 84 transgenic events, were analyzed to determine the expression profiles of the 10 candidate promoter sequences. Cassava source leaves, stems, and storage root samples were analyzed using quantitative RT-PCR and the results were summarized for each promoter (FIG. 1). The field trials revealed a very strong source leaf expression, although with large variation, for the transcripts controlled by the promoters of pSlRBCS2 (SEQ ID NO: 19) and pAtRBCS1A (SEQ ID NO: 18). Their transcripts were approximately 4-5 times more abundant than the transcript controlled by the next strongest leaf promoter pAtCAB1 (SEQ ID NO: 6). High abundance in source leaves was also observed for the transcripts controlled by pAtGAPA (SEQ ID NO: 12) and pStLS1 (SEQ ID NO: 15). Moderate transcript abundance in source leaves was detected for pMeGBSS1 (SEQ ID NO: 14) and pAtRBCS3B (SEQ ID NO: 20). Low levels in source leaves were found for the transcripts controlled by pStSSS3 (SEQ ID NO: 21) and pAtFBA2 (SEQ ID NO: 10), while no transcripts were found for pStSTP1 (SEQ ID NO: 22). In the heterotrophic organs, moderate to low levels were determined for the transcripts controlled by pMeGBSS1 (SEQ ID NO: 14), pStSSS3 (SEQ ID NO: 21), and pStSTP1 (SEQ ID NO: 22). Low levels were found for pAtRBCS1A (SEQ ID NO: 18) and residual levels were found in heterotrophic tissues for the transcripts controlled by the leaf-promoters pSlRBCS2 (SEQ ID NO: 19), pStLS1 (SEQ ID NO: 15), and pAtFBA2 (SEQ ID NO: 10).

Based on the transcript abundance observed in the different tissue (FIG. 1), the pSlRBCS2 and pAtRBCS1A promoters (SEQ ID NO: 19 and 18) appear to be very active in source leaves, although pAtRBCS1A (SEQ ID NO: 18) seems to have a low-level activity in sink organs, as well. The AtCAB1 (SEQ ID NO: 6) and AtGAPA (SEQ ID NO: 12) promoters were characterized by high and very specific source leaf expression, while the pAtRBCS3B (SEQ ID NO: 20) and pAtFBA2 promoters (SEQ ID NO: 20 and SEQ ID NO: 10) appeared rather weak. The StLS1 promoter (SEQ ID NO: 15) also showed weak activity in source leaves with additional residual activity in sink organs. The MeGBSS1 (SEQ ID NO: 14), StSSS3 (SEQ ID NO: 21), and StSTP1 (SEQ ID NO: 22) promoters were expected to be specific for heterotrophic organs. However, the abundance of transcripts controlled by the promoters of MeGBSS1 (SEQ ID NO: 14) and StSSS3 (SEQ ID NO: 21) was comparable between the three tissues tested. In these experiments, only pStSTP1 (SEQ ID NO: 22) seems to have a specific activity for heterotrophic organs (FIG. 1). According to the PCR results, all three of these promoter sequences resulted in rather weak activity.

### Example 2: Analyzing the Activity and Tissue Specificity of Promoters in Cassava Plants

Nineteen different promoter-GUS constructs were analyzed for their expression patterns in cassava plants. In brief, all promoter-GUS constructs were created using Golden Gate cloning (Engler et al. 2014). The promoters of AtCAB1 (SEQ ID NO: 6), pSlRBCS2 (SEQ ID NO: 19), AtRBCS3B (SEQ ID NO: 20), and pStLS1 (SEQ ID NO: 15) were taken from the “MoClo Plant Parts Kit” (Engler et al. 2014). All other promoter elements were created by either PCR amplification or DNA synthesis. The promoters of AtCAB1 (SEQ ID NO: 6), AtGAPA (SEQ ID NO: 12), AtFBA2 (SEQ ID NO: 10), AtRBCS3B (SEQ ID NO: 20), MeGBSS1 (SEQ ID NO: 14), StB33 (SEQ ID NO: 23), StFBPasecyt (SEQ ID NO: 8), StLS1 (SEQ ID NO: 15), StSSS3 (SEQ ID NO: 21), and StSTP1 (SEQ ID NO: 22) were maintained in level 0 promoter modules (GGAT-TACT). The promoters of AtSUC2 (SEQ ID NO: 24), CmGolS1 (SEQ ID NO: 11), CoYMV (SEQ ID NO: 7), DjDIO3 (SEQ ID NO: 9), IbSRD1 (SEQ ID NO: 16), MeGPT (SEQ ID NO: 2), MePsbr (SEQ ID NO: 3), AtRBCS1A (SEQ ID NO: 18), MeSUS1 (SEQ ID NO: 5), MeSWEET1-like (SEQ ID NO: 1), StGBSS1 (SEQ ID NO: 13), and StPat (SEQ ID NO: 17) were maintained in level 0 promoter+5′UTR modules (GGAT-AATG). All level 0 promoter modules (GGAT-TACT) were fused with the Tobacco mosaic virus 5′UTR (pICH41402; Engler et al. (2014)), a modified Beta-Glucuronidase coding sequence (“GUSPlus”; Broothaerts et al. (2005)), the E. coli Nopaline Synthase 3′UTR+terminator (pICH41421; Engler et al. (2014)), and the level 1-1f acceptor (pICH47732; Engler et al. (2014)) to create the respective promoter-reporter cassette. All level 0 promoter+5′UTR modules (GGAT-AATG) were fused with a modified Beta-Glucuronidase coding sequence (“GUSPlus”; Broothaerts et al. (2005)), the E. coli Nopaline Synthase 3′UTR+terminator (pICH41421; Engler et al. (2014)), and the level 1-1f acceptor (pICH47732; Engler et al. (2014)) or level 1-3f acceptor (pICH47751; Engler et al. (2014)) to create the respective promoter-reporter cassette. The level 1 plasmids containing the respective promoter-reporter cassettes were transferred into the transformation vector p134GG (Mehdi et al., 2019) to create the final level 2 transformation plasmids. Cassava plants (genotype 60444) were transformed with promoter-reporter constructs as described previously (Bull et al., 2009). Hygromycin-resistant transformants were screened by ß-glucuronidase histological staining. Plants with clear GUS staining were maintained in tissue culture and successively analyzed for their tissue specific expression patterns.

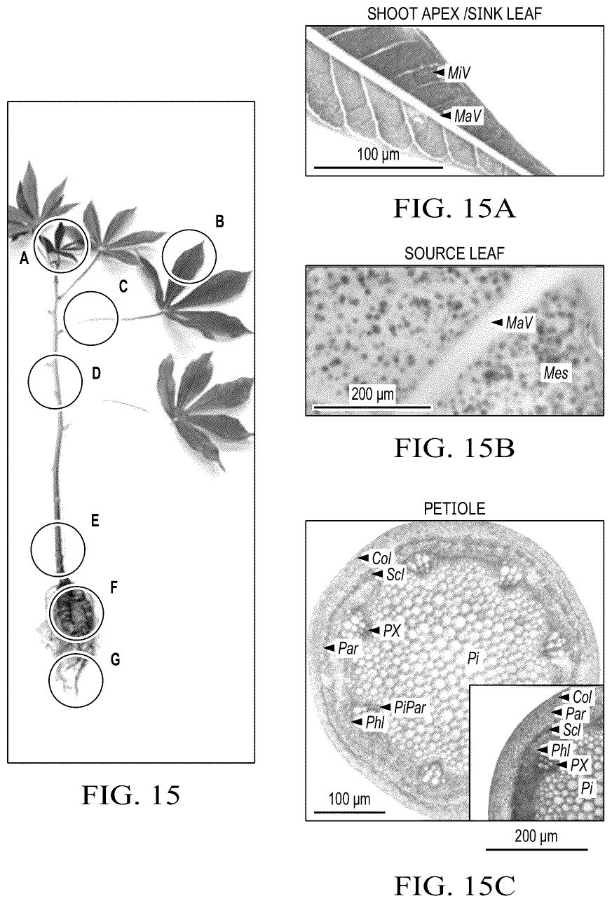

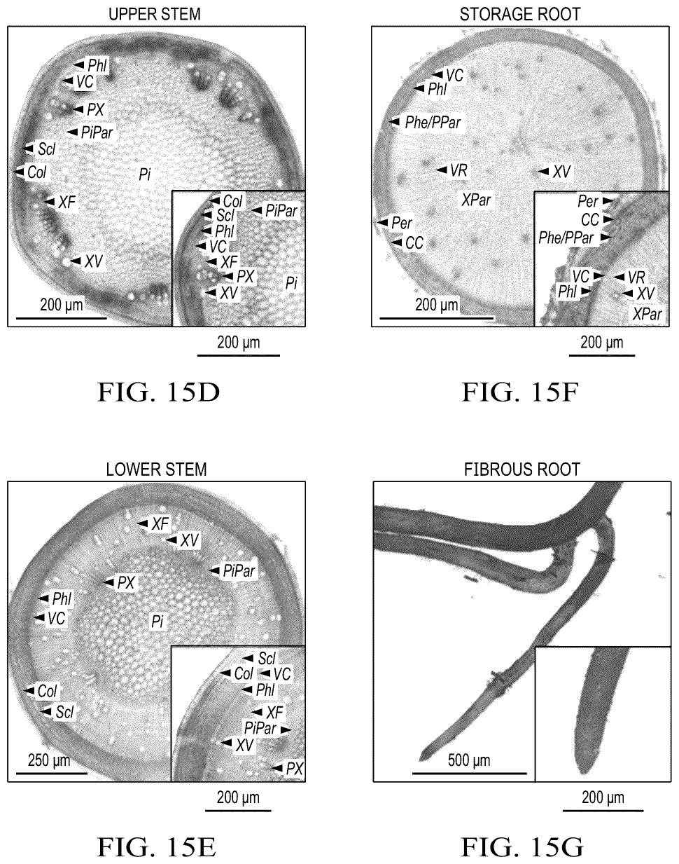

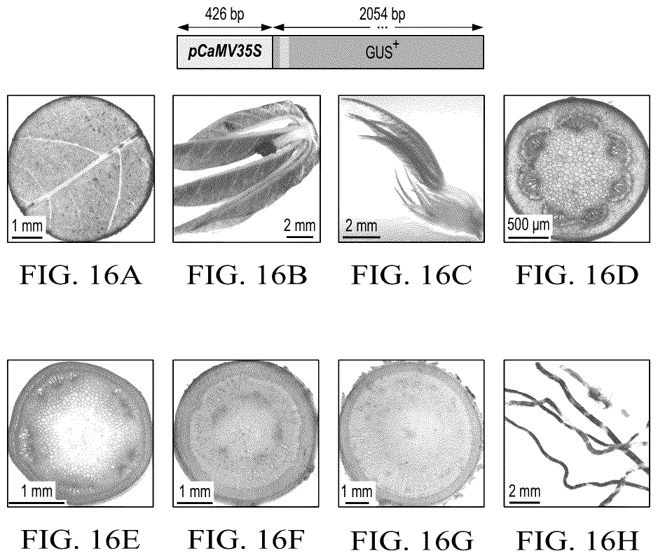

For the analysis of these promoter-GUS plants, up to seven different tissues were sampled and subjected to staining and microscopy. Emerging leaves, developing leaves, fully developed leaves, petioles, upper stem sections, lower stem sections, storage root sections, and fibrous roots (FIG. 15). Emerging and developing leaves are characterized by brownish color and were termed “sink” leaves (defined as leaves that have a net import of carbon), while green, fully expanded leaves were considered “source” leaves (defined as leaves with net export of carbon).

For staining, cassava tissues were sampled into ice-cold 90% acetone solution. Leaf-samples were taken with a leaf puncher and cross-sections were manually prepared with a razor blade. These sections were covered with GUS staining buffer (200 mM NaP pH7, 100 mM K3[Fe(CN6)], 100 mM K4[Fe(CN6)], 500 mM EDTA, 0.5% SILWET® gold) and thoroughly vacuum infiltrated for 10 minutes. The GUS staining buffer was removed and replaced with fresh GUS staining solution containing GUS staining buffer with 0.75 mg/ml 5-bromo-4-chloro-3-indolyl-β-D-glucuronic acid (X-Gluc; pre-dissolved in a small amount of DMSO). The GUS staining solution was thoroughly vacuum infiltrated for 10 minutes. The infiltrated tissues were incubated at a temperature of 37° C. overnight or stopped shortly after incubation in case of very quick staining (e.g. pCoYMV, pDjDIO3). After removal of the GUS staining solution, 70% ethanol was added to the tissue sections and incubated at a temperature of 37° C. until the tissues were cleared. Light microscopic images were taken on a Zeiss Axioskop or a Zeiss STEMI SV11 Stereomicroscope (Zeiss, Wetzlar, Germany).

For quantification of GUS expression, RNA extraction of cassava source leaves and storage roots was performed using the Spectrum Plant Total RNA Kit (Sigma-Aldrich, St. Louis, MO, USA). cDNA was generated from 0.2-1 μg of RNA using RevertAid H Minus Reverse Transcriptase as indicated by the manufacturer (Thermo Fisher Scientific, Waltham, MA, USA). The cDNA was diluted 1:10 and quantification of gene expression was examined using GoTaq® qPCR Master Mix (Promega, Madison, WI, USA). The assay was mixed in a 96-well plate and measured in an AriaMx Real-time PCR System (Agilent, Santa Clara, CA, USA). The normalized GUS expression of the promoter::GUS lines was determined by the 2^{-ΔCt} calculation method with MeGAPDH (Manes.06g116400) as a reference gene. The normalized GUS expression of the respective promoter::GUS lines was calculated in relation to the normalized expression of the pCaMV35S::GUS lines and displayed as relative expression pCaMV35S::GUS lines in percent to provide an approximate classification of expression strength.

The plants comprising reporter constructs displayed GUS staining in different tissues and cell types as described in detail in the following Examples. In order to more accurately define these different cell types, counterstaining with toluidine blue was performed. As shown in FIG. 15, “source” and “sink” leaves can easily be divided into vascular bundles and mesophyll cells (FIG. 15A-B). In petioles, the collenchyma, the sclerenchyma, the phloem, protoxylem/xylem parenchyma, pith parenchyma, and the pith cells can be differentiated from outside to inside (FIG. 15C). Stem tissues are characterized by collenchyma, sclerenchyma, phloem, vascular cambium, and varying degrees of secondary xylem and pith tissue, depending on the position of the stem (FIG. 15D-E). Especially the lower, heterotrophic stem tissues display increasing levels of secondary xylem tissues, consisting of xylem fibers, water-transporting xylem vessels, and starch-storing xylem parenchyma cells (FIG. 15E). Storage roots have periderm tissue, the cork cambium, phelloderm/phloem parenchyma, phloem, vascular cambium, and xylem cells from outside to inside. Alongside xylem vessels, the xylem tissue is mostly dominated by starch-storing xylem parenchyma cells in storage roots (FIG. 15F). Lower stems and storage roots, both heterotrophic starch-storing tissues, are overall similar and both tissues are characterized by many vascular rays, ensuring the connection of assimilate- and water transport systems, despite the increasing distance through the formation of secondary xylem during secondary growth (FIG. 15E-F).

### Example 3: Analysis of GUS Expression Driven by Selected Promoters from the Field-Tested Transgenic Plants

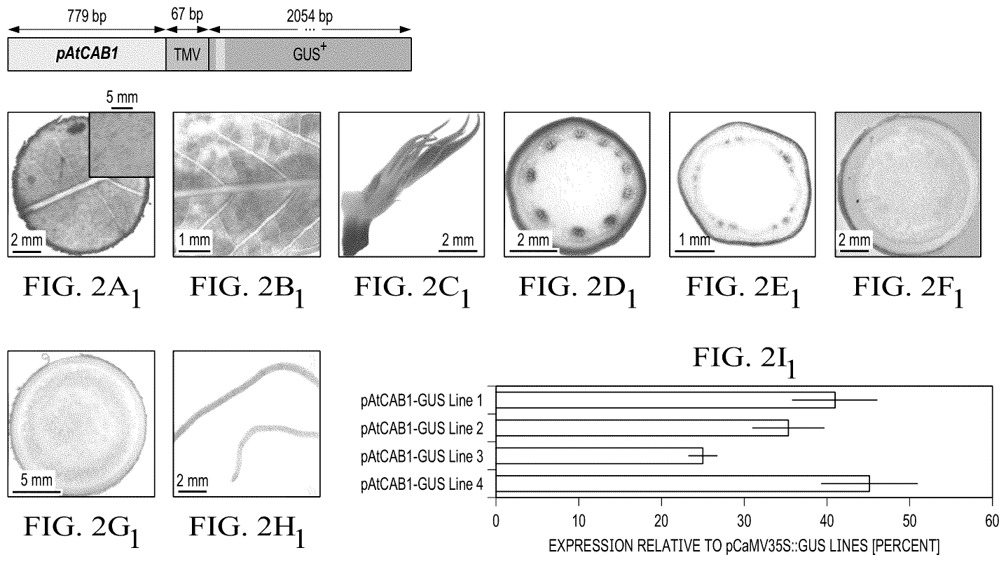

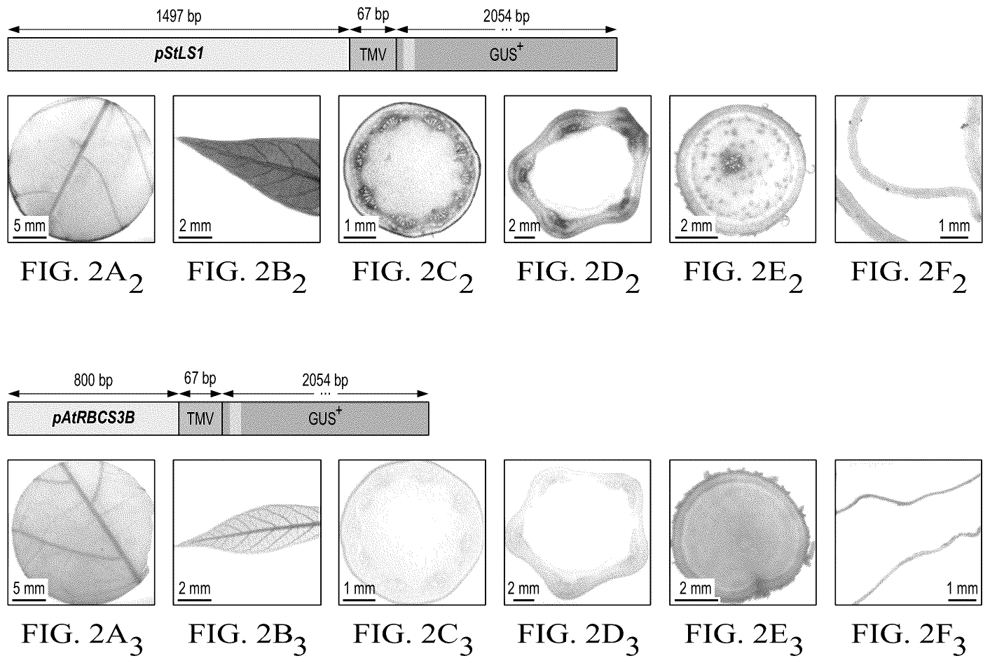

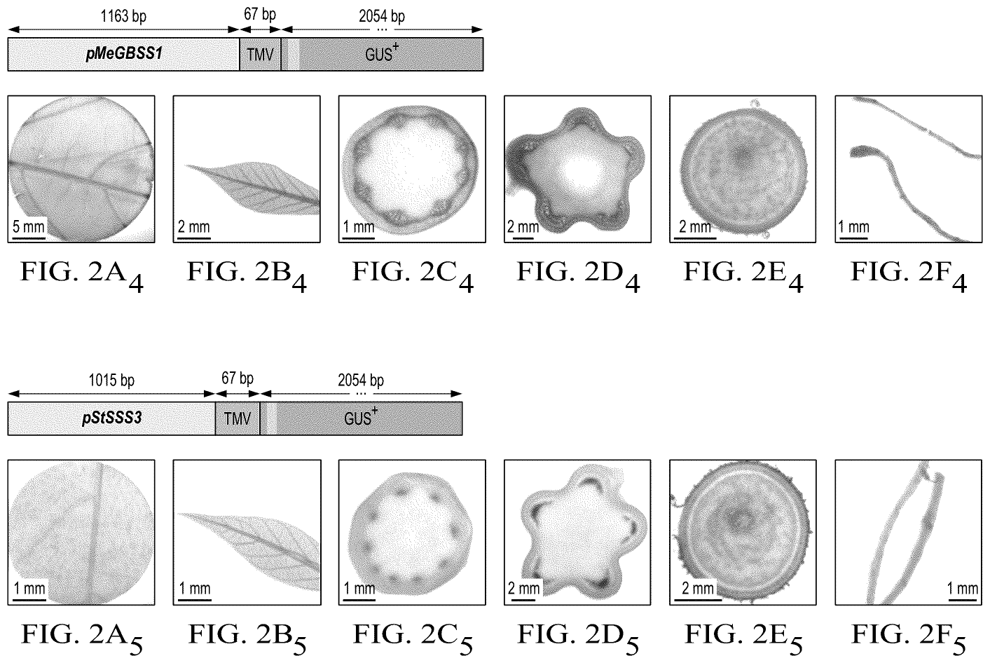

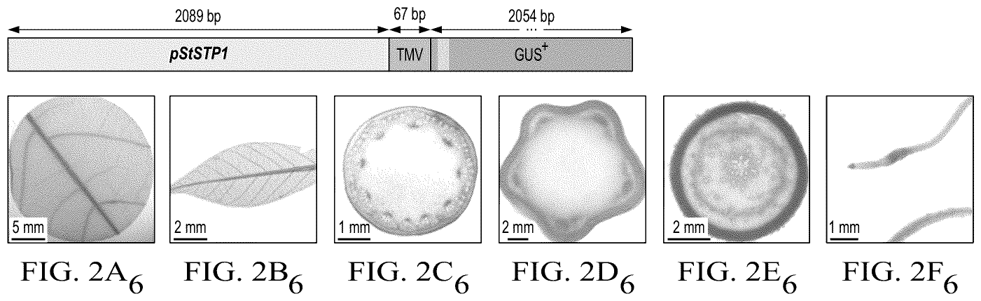

For six of the ten promoters (pAtCAB1, pStLS1 (SEQ ID NO: 15), pAtRBCS3, pMeGBSS1 (SEQ ID NO: 14), pStSSS3 (SEQ ID NO: 21), pStSTP1 (SEQ ID NO: 22)) included in the multigene construct plants and tested for their gene expression in the field (Example 1; FIG. 1), dedicated promoter-GUS plants were also created as described (Example 2; FIG. 2).

In the pAtCAB1::GUS events, staining was observed in the mesophyll of source leaves, sink leaves and newly emerging leaves (FIG. 2A₁-C₁). Petiole and upper stem cross-sections displayed staining in collenchyma, outer parenchyma, and protoxylem areas (FIG. 2D₁-E₁). Lower stem sections, storage roots and fibrous roots were completely devoid of GUS staining (FIG. 2F₁-H₁). Therefore, these results demonstrate that the promoter element of AtCAB1 (SEQ ID NO: 6) can drive expression in autotrophic cassava tissues without activity in heterotrophic plant parts. To determine the approximate expression strength of these promoter elements in the reporter plants, the relative expression level of the different lines was determined and compared to the relative expression levels of pCaMV35 as determined in three pCaMV35S::GUS lines. The promoter of CaMV35S is ubiquitously active in cassava as well (FIG. S2) and its expression strength was used as a tangible reference point. The promoter of AtCAB1 (SEQ ID NO: 6) showed approximately 25% to 45% activity compared to the promoter element of CaMV35S, respectively (FIG. 2I₁). Because pCaMV35S is a well-documented, strong promoter, the promoter of AtCAB1 (SEQ ID NO: 6) can drive specific and reasonably strong expression in the autotrophic tissues of cassava, which is in line with the field expression results displayed in FIG. 1.

The promoter of pStLS1 (SEQ ID NO: 15) displayed the expected staining in the source- and sink leaves (FIG. 2A₂-B₂). However, it also displayed staining in phloem and xylem tissues of petioles, stems and storage roots (FIG. 2C₂-E₂). Only the fibrous roots were devoid of staining (FIG. 2F₂). This staining pattern matches the expression results (FIG. 1), demonstrating that pStLS1 (SEQ ID NO: 15) has activity in both source- and sink tissues in cassava.

Similar to the low transcript levels observed for pAtRBCS3B (SEQ ID NO: 20; FIG. 1), rather faint staining pattern were observed for pAtRBCS3B::GUS. Staining was seen in source- and sink leaves (FIG. 2A₃-B₃), as well as, unexpectedly, in the storage root cambium region (FIG. 2E₃). It seems that pAtRBCS3B (SEQ ID NO: 20) is not amendable strong or specific expression in cassava.

The promoter of pMeGBSS1 (SEQ ID NO: 14) was expected to be sink specific (Koehorst-van Putten et al., 2012). However, activity in both source- and sink tissue was observed. Source leaves (FIG. 2A₄) and sink leaves (FIG. 2B₄) were stained, and strong staining was seen in the phloem area of the petiole (FIG. 2C₄). Besides the stem pith, all cell types of stems and storage roots were stained (FIG. 2D₄-E₄). Fibrous roots were devoid of staining (FIG. 2F₄). Although the staining in stems and storage roots appears to be stronger compared to the other tissues, the inferred promoter activity from the expression results (FIG. 1) suggest a rather equal activity between source- and sink. In any case, the promoter was not storage root specific in these experiments.

In contrast to the expression results from the field-tests (FIG. 1), indicating comparable source- and sink activity for the StSSS3 promoter (SEQ ID NO: 21), the pStSSS3::GUS plants displayed a staining specific for heterotrophic tissues. Staining was observed in the protoxylem of petioles (FIG. 2C₅) and stems (FIG. 2D₅), in the phloem and xylem areas of the storage root (FIG. 2E₅), but not in the fibrous roots (FIG. 2F₅). This may be because the promoters used in the multigene constructs (FIG. 1) can potentially be influenced by neighboring promoters.

The promoter of StSTP1 (SEQ ID NO: 22) displayed a weak but sink-specific behavior in the multigene construct plants (FIG. 1). A matching staining pattern was observed in the promoter-GUS plants. The activity in source- and sink leaves was confined to the vasculature (FIG. 2A₆-B₆). Petioles showed staining in the protoxylem and outside the sclerenchyma (FIG. 2C₆). While fibrous roots displayed no staining besides the root tip (FIG. 2F₆), most staining was observed in the stems (FIG. 2D₆) and storage roots (FIG. 2E₆). Although the activity seems limited, pStSTP1 (SEQ ID NO: 22) can mediate a rather sink specific expression.

### Example 4: Analysis of Promoter Sequences for Expression in Autotrophic Tissues

As described in the preceding Examples, several of the promoter elements tested can mediate a specific expression pattern for autotrophic tissues in cassava plants (e.g. pSlRBCS2 (SEQ ID NO: 19), pAtCAB1 (SEQ ID NO: 6)) and some are able to mediate a rather specific expression pattern for heterotrophic organs (e.g. pStSSS3, pStSTP1). However, none of these promoters appeared to be particularly strong and specific for the sink tissues. Therefore, 13 additional reporter lines comprising promoter-GUS constructs, having promoters with potential transport and heterotrophic storage tissues specificity were created (Table 2).

TABLE 2
Summary of 13 additional promoter sequences analyzed in cassava

| SEQ ID | | | Gene/NCBI | Analyzed | Results |
|---|---|---|---|---|---|
| NO: | Promoter | Code | identifier | by | in FIG. |
| 1 | BIDIRECTIONAL | MeSWEET1 | Manes.18G086400 | Histology | 8 |
| | SUGAR | | | | |
| | TRANSPORTER | | | | |
| | SWEET1 | | | | |
| 2 | GLUCOSE-6- | MeGPT | Manes.16G010700 | Histology + | 12 |
| | PHOSPHATE/ | | | qPCR | |
| | PHOSPHATE | | | | |
| | TRANSLOCATOR | | | | |
| 3 | PHOTOSYSTEM | MePsbR | Manes.15G102500 | Histology + | 3 |
| | II SUBUNIT R | | | qPCR | |
| 4 | pManes.14g071100 | pManes.14g071100 | pManes.14g071100 | Histology | 13 |
| 5 | SUCROSE | MeSUS1 | Manes.03g044400 | Histology + | 8 |
| | SYNTHASE 1 | | | qPCR | |
| 7 | COMMELINA | CoYMV | X52938.1 | Histology | 7 |
| | YELLOW | | | | |
| | MOTTLE | | | | |
| | VIRUS | | | | |
| 8 | CYTOSOLIC | StFBPase_{cyt} | LOC102589275 | Histology | 17 |
| | FRUCTOSE-1,6- | | | | |
| | BISPHOSPHATASE | | | | |
| 9 | DISCORIN 3 | DjDio3 | GU324672.1 | Histology | 18 |
| | SMALL SUBUNIT | | | | |
| 11 | GALACTINOL | GolS1 | AF249912.2 | Histology | 5 |
| | SYNTHASE 1 | | | | |
| 13 | GRANULE-BOUND | StGBSS1 | X58453.1 | Histology + | 11 |
| | STARCH SYNTHASE1 | | | qPCR | |
| 16 | MADS-BOX | IbSRD1 | ACN39597.1 | Histology | 14 |
| | PROTEIN SRD1 | | | | |
| 17 | PATATIN CLASS 1 | StPat | GQ352473 | Histology + | 9 |
| | | | | qPCR | |
| 23 | B33 GENE | StB33 | X14483.1 | Histology + | 10 |
| | | | | qPCR | |
| 24 | SUCROSE-PROTON | AtSUC2 | AT1G22710 | Histology | 4 |
| | SYMPORTER 2 | | | | |

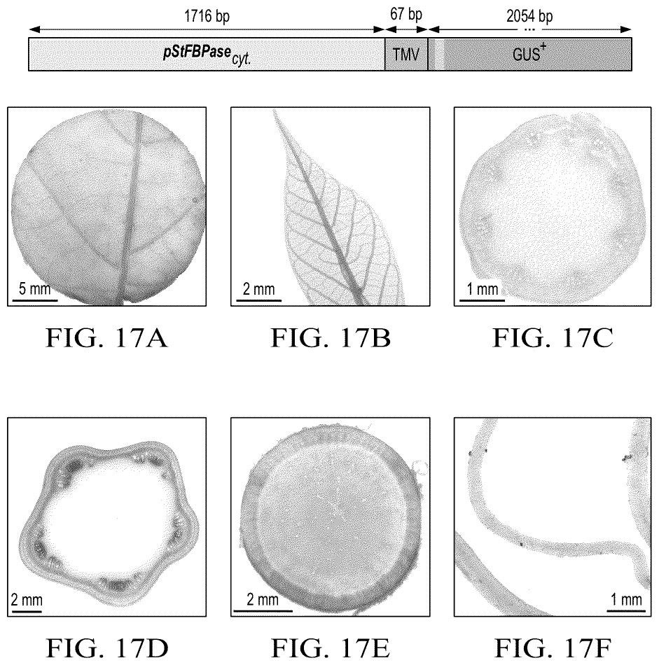

Two additional promoter-GUS constructs were created, one including the promoter of the cytosolic fructose-1,6-bisphosphatase StFBPase_{cyt} (SEQ ID NO: 8) and the other including the promoter of MePsbr (SEQ ID NO: 3). The promoter of StFBPase_{cyt} (SEQ ID NO: 8) and the promoter of MePsbR (SEQ ID NO: 3) were chosen as candidates based on RNA transcript data. In contrast to the expected mesophyll-specific staining pattern, pStFBPase_{cyt} (SEQ ID NO: 8) showed considerable staining in the phloem- and cambium areas of stems (FIG. 17D) and storage roots (FIG. 17E), in addition to staining in the mesophyll of source (FIG. 17A) and sink leaves (FIG. 17B).

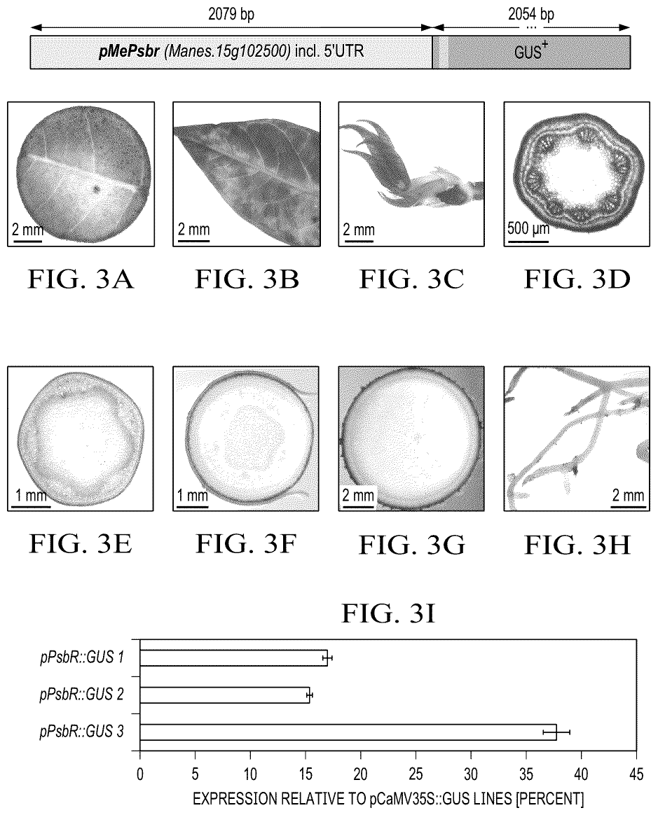

However, a very specific staining pattern was found for pMePsbR (SEQ ID NO: 3; FIG. 3). Here, staining was observed in the mesophyll of source leaves, sink leaves and newly emerging leaves (FIG. 3A-C). Petiole cross-sections displayed labeling in most cell types beside sclerenchyma and pith tissue (FIG. 3D) and upper stem sections displayed staining in the pith parenchyma, the phloem and cambium area, and the collenchyma (FIG. 3E). The heterotrophic lower stem sections, storage roots and fibrous roots were completely devoid of GUS staining (FIG. 3F-H).

Therefore, the results described herein show for the first time that the promoter of MePsbR (SEQ ID NO: 3) can drive specific expression of heterologous transcribable polynucleotides in autotrophic cassava tissues. To determine the approximate expression strength of pMePbsbR, we determined the relative expression level of the different lines and compared them to the relative expression levels of pCaMV35 as determined in three pCaMV35S::GUS lines. The promoter of MePsbR (SEQ ID NO: 3) showed approximately 15% to 35% activity compared to the promoter element of CaMV35S (FIG. 3I). Similar expression levels were obtained for pAtCAB1 (SEQ ID NO: 6) and because pCaMV35S is a well-documented, strong promoter, the promoter of MePsbR (SEQ ID NO: 3) can drive specific and reasonable strong expression in the autotrophic tissues of cassava.

### Example 5: Analysis of Promoter Sequences for Expression in Phloem Tissues

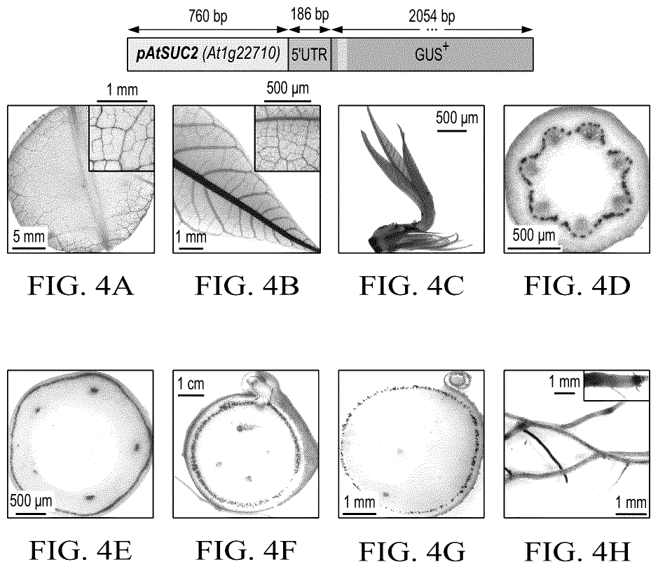

The promoter of AtSUC2 (SEQ ID NO: 24) was selected and expected to be phloem specific in cassava, because this promoter has been used as a phloem-specific tool in numerous studies in different species (Stadler and Sauer, (2019). Indeed, pAtSUC2::GUS lines displayed pronounced staining in the minor and major veins of source leaves (FIG. 4A), sink leaves (FIG. 4B), newly developing leaves (FIG. 4C), as well as a dotted staining in the phloem area of petioles (FIG. 4D), upper stem (FIG. 4E), lower stem (FIG. 4F), and storage roots (FIG. 4G). The dotted GUS staining in the phloem very likely results from the staining of phloem companion cells. The vasculature of fibrous roots and the root tips also displayed GUS staining (FIG. 4H). In addition, some staining was observed in protoxylem and xylem parenchyma areas (FIG. 4D-F). These results demonstrate that pAtSUC2 (SEQ ID NO: 24) is well suited to drive phloem companion cell specific expression also in cassava.

Two additional phloem promoters were chosen for testing, a promoter sequence of Cucumis melo driving expression of the GALACTINOL SYNTHASE1 (pGolS1; SEQ ID NO: 11) and a sequence from Commelina Yellow Mottle Virus (pCoYMV; SEQ ID NO: 7). The former sequence was previously described to have specific activity for the loading phloem, because GUS staining was specifically observed in the smallest veins of the source leaves (Haritatos et al., 2000). The later promoter sequence was described as a promoter with high-level expression, specific to phloem cells, as well as phloem-associated cells (Medberry et al., 1992). In addition, GUS staining was seen in phloem unloading tissues, like the tapetum (Medberry et al., 1992).

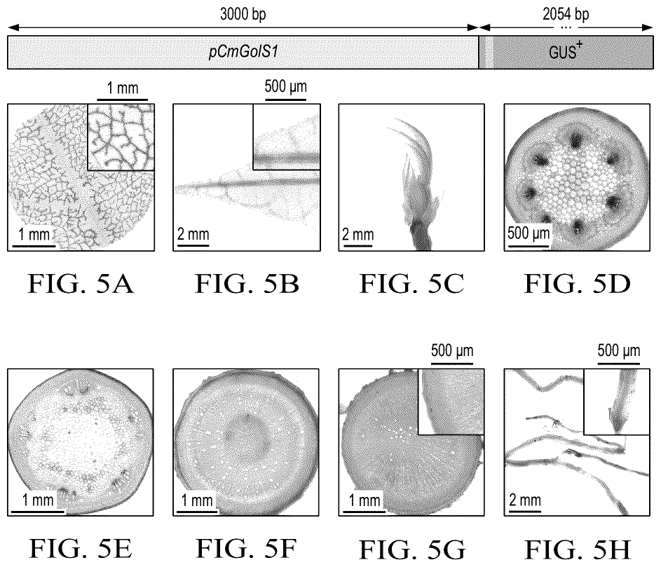

GUS staining of transgenic pCmGolS1::GUS plant lines revealed specific staining of minor veins in the source leaves in cassava (FIG. 5A), matching the results obtained in previous publications (Haritatos et al., 2000). The majority of lines also displayed slightly patchy staining in the veins of sink and newly emerging leaves (FIG. 5B-C), staining in the protoxylem/xylem parenchyma of petioles (FIG. 5D) and green stems (FIG. 5E), as well as slight staining in the pith tissue of auto- and heterotrophic stem tissue (FIG. 5E-F). While storage roots displayed very little staining (FIG. 5G), fibrous roots also displayed a slightly patchy staining (FIG. 5H). Overall, the promoter sequence used appeared mostly active in minor veins of source leaves but also appeared to convey some activity in non-phloem-related tissues in cassava. Despite the activity outside the leaf, the promoter could still be a useful tool for biotechnological approaches centered on phloem loading.

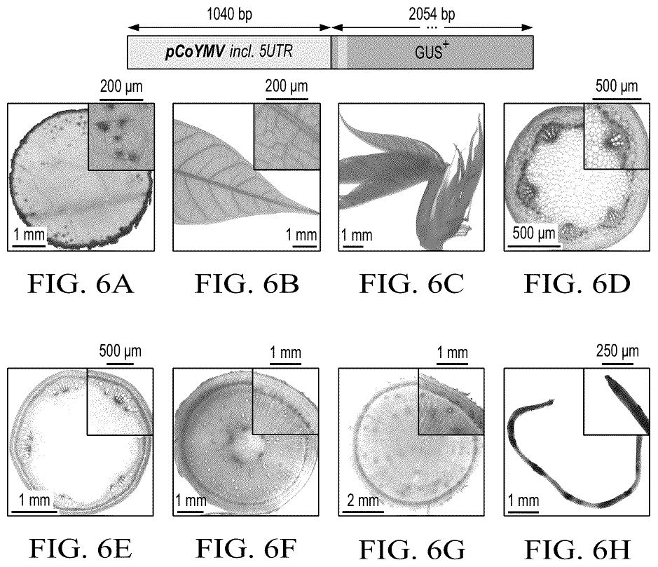

In contrast to the pCmGolS1::GUS plant lines, which showed preferential activity in the loading phloem, the pCoYMV::GUS plant lines appeared to be more specific toward the transport- and unloading phloem. None of the lines studied showed any staining of source leaf vasculature, but rather displayed a staining pattern that appeared wound induced, due to the staining of the cutting site, as well as the punctual staining within the mesophyll (FIG. 6A) or in fibrous roots (FIG. 6H). In the sink leaves, the staining was observed just outside the vasculature, potentially representing the phloem parenchyma (FIG. 6B-C). In addition to some staining in protoxylem and pith parenchyma (FIG. 6D-F), pronounced staining was observed in the phloem tissues of petioles (FIG. 6D), autotrophic stems (FIG. 6E), heterotrophic stems (FIG. 6F), and storage roots (FIG. 6G). Interestingly, tissues with important functions in lateral transport, as indicated by the staining of vascular rays in the lower stems and storage roots, were also stained in these promoter-reporter plants (FIG. 6F-G). Taken together, the analyzed sequence of pCoYMV is rather specific towards transport and unloading phloem tissues, which is in line with previous results, showing promoter activity in vascular and reproductive tissues (Medberry et al., 1992). Although not a quantitative measure, all pCoYMV::GUS lines stained within seconds of adding staining buffer, indicating a very strong activity for transport and unloading phloem tissues.

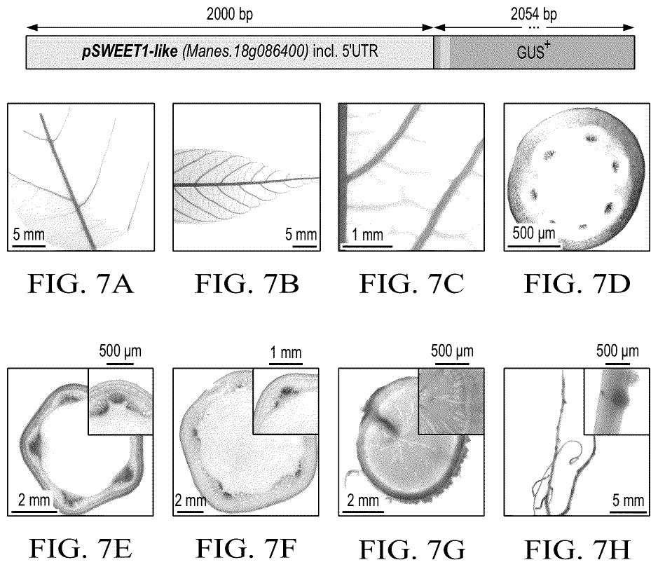

The promoter of pMeSWEET1-like (SEQ ID NO: 1; FIG. 7) also displayed staining in the phloem areas, although less specific compared to pAtSUC2 (SEQ ID NO: 24; FIG. 4). The promoter element of MeSWEET1-like (SEQ ID NO: 1) was selected as a candidate based on RNA transcript data. Promoter-GUS lines comprising this promoter element revealed staining in the vasculature of source- and sink leaves (FIG. 7A-B), as well as staining in phloem and parenchyma tissues of petioles and stems (FIG. 7D-G). The outer storage root region, containing phloem and phloem parenchyma, displayed pronounced GUS staining (FIG. 7G). In addition, the pMeSWEET1-like promoter (SEQ ID NO: 1) showed activity in the fibrous root vasculature and root tips (FIG. 7H). As first disclosed herein, these results indicate that pMeSWEET1-like promoter (SEQ ID NO: 1) has preferential activity in phloem and parenchyma cells in cassava.

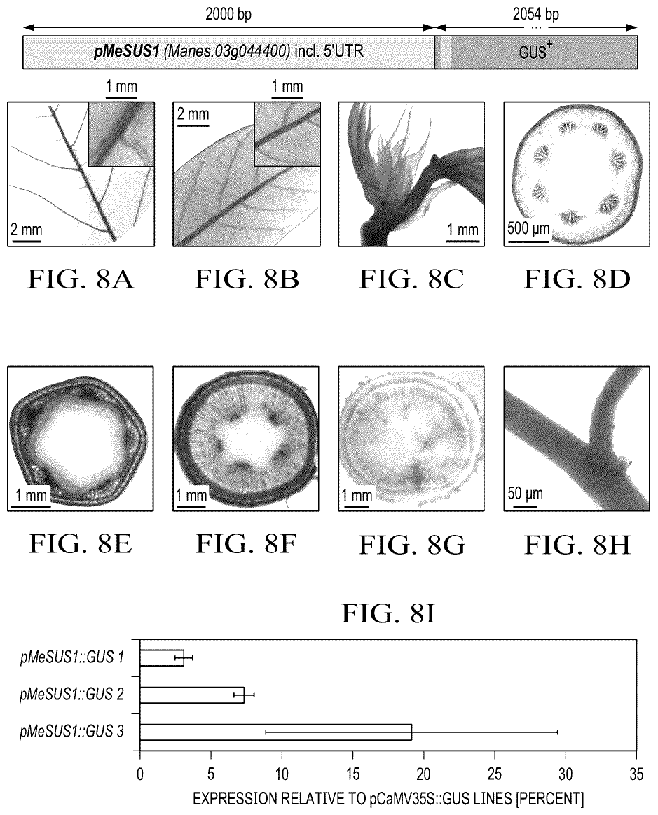

The promoter of MeSUS1 (SEQ ID NO: 5) was chosen as a candidate for testing as a putative phloem promoter based on RNA transcript data. The pSUS1::GUS lines displayed an interesting staining pattern, resembling the pCoYMV promoter (FIG. 6). pSUS1 was active in the major veins of the leaf vasculature (FIG. 8A-B), in phloem and parenchyma cell types (FIG. 8D-G), and in fibrous root vasculature (FIG. 8H). It displayed pronounced staining in vascular rays of stems and storage roots (FIG. 8F-G) and the staining pattern in the storage roots indicated preferential activity in the phloem unloading area, as well as in young xylem cells of the storage roots (FIG. 8G). This staining pattern matches the previously described symplasmic unloading mode of cassava and the previously observed metabolic gradients within the storage root.

To determine the approximate expression strength of pMeSUS1 (SEQ ID NO: 5), tested the relative expression level of the different lines was tested and compared them to the relative expression levels of pCaMV35 as determined in three pCaMV35S::GUS lines. The promoter elements of MeSUS1 (SEQ ID NO: 5) showed approximately 5-20% activity compared to the promoter element of CaMV35S (FIG. 8I). Although its expression is considerably weaker than the expression strength of the more parenchyma-dominated promoters described in Example 6, the MeSUS1 (SEQ ID NO: 5) promoter is active in far less cells across the storage root, thinning out the specific signal. Overall, these results demonstrate for the first time that the promoter of pMeSUS1 (SEQ ID NO: 5) can be used as a promoter for applications focused on phloem transport and unloading.

### Example 6: Analysis of Promoter Sequences for Expression in Heterotrophic Storage Tissues

Storage root specific promoters are of special interest for cassava. However, very few storage root-specific promoters in cassava are known in the art. One notable example being a promoter sequence from the potato PATATIN CLASS I promoter (pStPat; SEQ ID NO: 17), coding for the tuber storage protein patatin, was previously shown to mediate this specific expression pattern in cassava. The promoter element of pStPat (SEQ ID NO: 17) was included in this study for confirmation of its tissue specificity and activity. In addition, the promoter elements of pStB33 (SEQ ID NO: 23), pStGBSS1 (SEQ ID NO: 13), pDjDIO3 (SEQ ID NO: 9), and pMeGPT (SEQ ID NO: 2) were selected for testing. The promoters of StB33 (SEQ ID NO: 23), StGBSS1 (SEQ ID NO: 13), and DjDIO3 (SEQ ID NO: 9) were previously understood to have preferential storage organ activity in other plants. The promoter of MeGPT (SEQ ID NO: 2) was chosen as a candidate based on RNA transcript data.

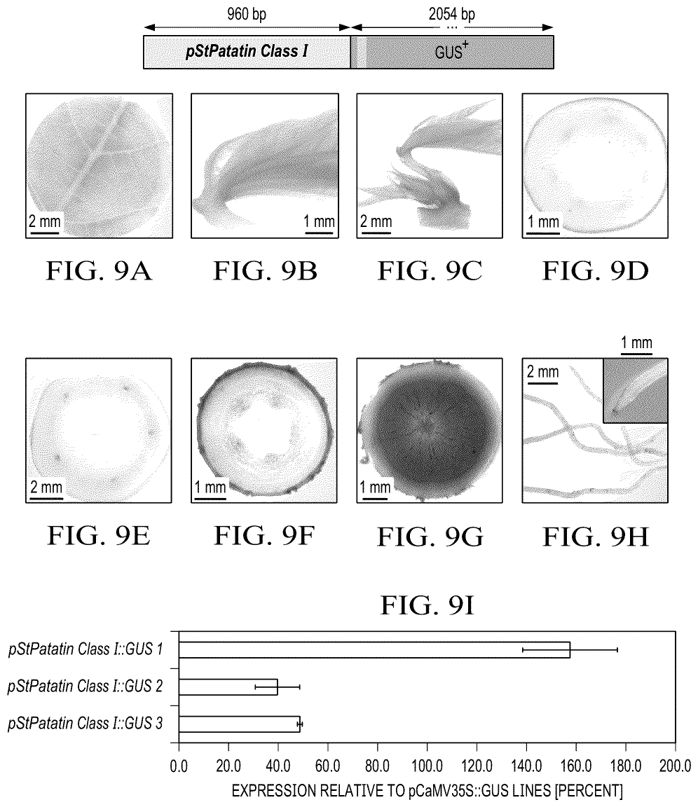

As expected, the promoter of pStPat (SEQ ID NO: 17) displayed strong expression in storage roots, as well as the highest specificity for storage root expression among all promoters tested. The lines displayed no staining in leaves and petioles (FIG. 9A-D), only faint staining in upper and lower stems (FIG. 9E-F), as well as no staining in fibrous roots (FIG. 9H). However, strong staining was observed in the xylem core area of the storage root (FIG. 9G), consisting mostly of xylem parenchyma cells. The relative expression level of pStPat, compared to the relative expression level of pCaMV35, was approximately 40-160%, depending on the respective line (FIG. 9I). These results underscore the storage root specificity of pStPat in cassava and confirm a high promoter activity in storage roots.

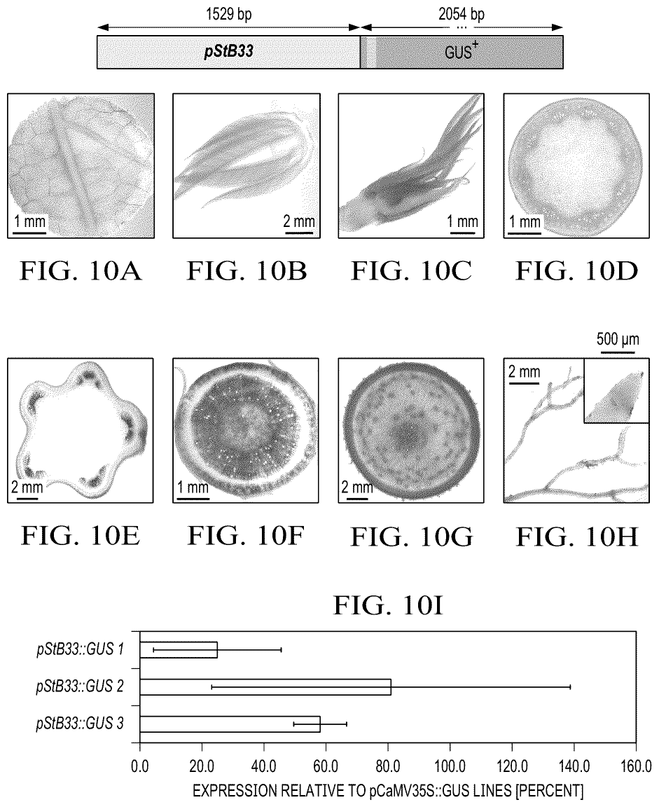

The promoter of StB33 (SEQ ID NO: 23), which is also part of the class I family of patatin genes, appeared very suitable to drive strong expression in heterotrophic storage tissues in cassava as well. The pStB33::GUS lines, displayed staining of minor veins in source leaves and no staining in sink leaves and petioles (FIG. 10A-D). Upper stem tissue showed staining of collenchyma and protoxylem (FIG. 10E), while the heterotrophic lower stem section (FIG. 10F) and storage roots displayed strong staining in xylem and phloem parenchyma (FIG. 10G). In addition, the vasculature and root tips of fibrous roots were stained (FIG. 10F). The relative expression level of pStB33 (SEQ ID NO: 23), compared to the relative expression level of pCaMV35, was approximately 20-80%, depending on the respective line. Therefore, pStB33 (SEQ ID NO: 23) is rather specific for sink tissues and has a high activity in sink organs.

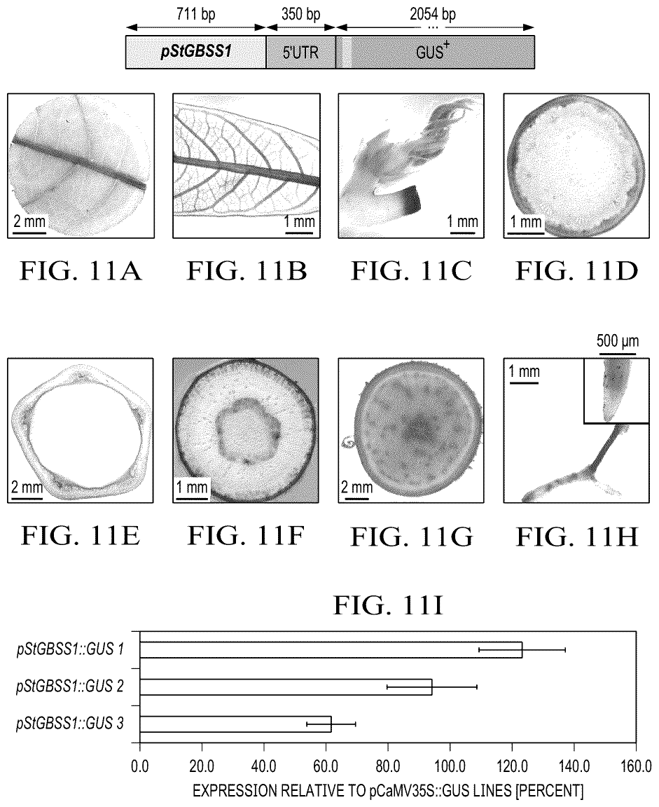

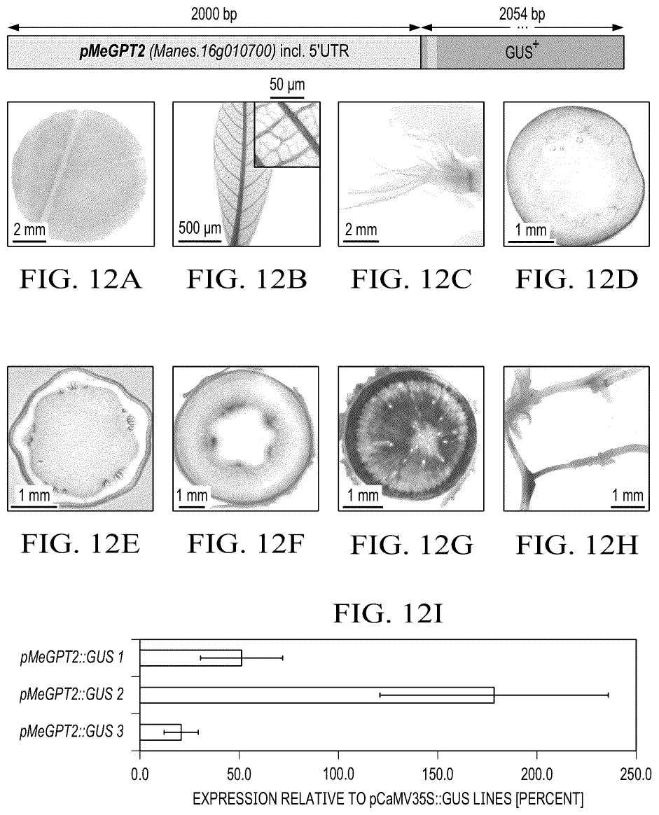

The pStGBSS1::GUS lines displayed a staining pattern with predominant activity in the phloem- and xylem parenchyma cells of storage roots (FIG. 11G). They also displayed staining in the collenchyma of petioles and stems (FIG. 11D-F), the pith parenchyma (FIG. 11E-F), and the vasculature of fibrous roots (FIG. 11H). In contrast to the two patatin promoters pStPat and pStB33 (SEQ ID NOs: 17 and 23; FIG. 9-10), pStGBSS1 (SEQ ID NO: 13) showed activity in both source- and sink leaf vasculature (FIG. 11A-B). The relative GUS expression level caused by pStGBSS1 (SEQ ID NO: 13), compared to the relative GUS expression level caused by pCaMV35, was approximately 60-120%, depending on the respective line (FIG. 11I). Therefore, pStGBSS1 (SEQ ID NO: 13) displays a similar sink activity as pStPat (SEQ ID NO: 17), but seems less specific due to its higher activity in some cell types of leaves, petioles and stems.

The pStGBSS1::GUS lines displayed a staining pattern with predominant activity in the phloem- and xylem parenchyma cells of storage roots (FIG. 11G). They also displayed staining in the collenchyma of petioles and stems (FIG. 11D-F), the pith parenchyma (FIG. 11E-F), and the vasculature of fibrous roots (FIG. 11H). In contrast to the two patatin promoters pStPat and pStB33 (SEQ ID NOs: 17 and 23; FIG. 9-10), pStGBSS1 (SEQ ID NO: 13) showed activity in both source- and sink leaf vasculature (FIG. 11A-B). The relative GUS expression level caused by pStGBSS1 (SEQ ID NO: 13), compared to the relative GUS expression level caused by pCaMV35, was approximately 60-120%, depending on the respective line (FIG. 11I). Therefore, pStGBSS1 (SEQ ID NO: 13) displays a similar sink activity as pStPat (SEQ ID NO: 17), but seems less specific due to its higher activity in some cell types of leaves, petioles, and stems.

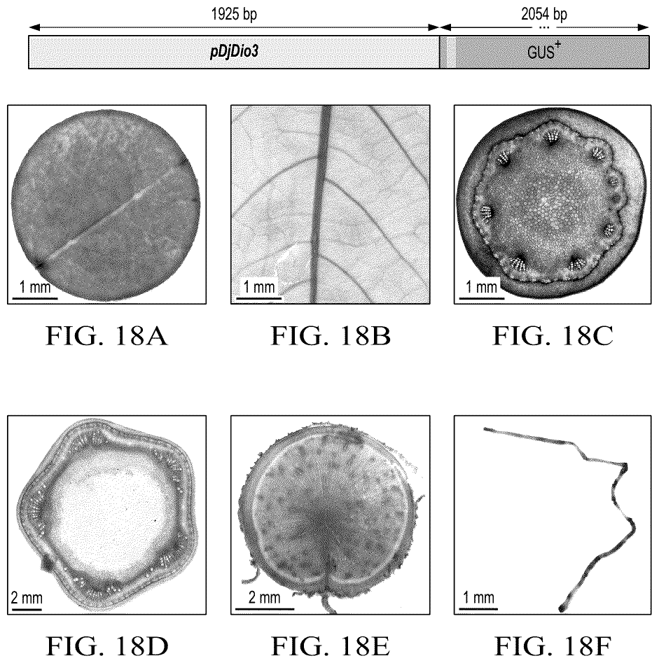

While the promoters of pStPat (SEQ ID NO: 17), pStB33 (SEQ ID NO: 23), pStGBSS1 (SEQ ID NO: 13), and pMeGPT (SEQ ID NO: 2) all show preferential activity in heterotrophic storage tissues, the promoter of the DIOSCORIN 3 SMALL SUBUNIT gene from Dioscorea japonica (DjDIO3) did not. In contrast to what was previously suggested in the art, pDjDI03::GUS lines displayed a rather ubiquitous staining pattern in cassava (FIG. 18).

### Example 7: Analysis of Promoter Sequences for Expression in Cambial Tissues

To achieve transgenic interventions targeting cassava secondary growth, promoters with distinct activity in the vascular cambium would be useful tools. Towards this end, the tissue specificity of the sweet potato MADS-box transcription factor pIbSRD1 (SEQ ID NO: 16) in cassava, a promoter that was previously characterized in thale cress, carrot, potato and sweet potato. In sweet potato, the SRD1 expression was shown to be auxin-responsive and the transcript was localized in the primary cambium, secondary cambium, and primary phloem cells. The main promoter activity in thale cress could be demonstrated in the vasculature including pericycle and endodermis, while the promoter activity was strong in all cells of carrot taproots and potato tubers.

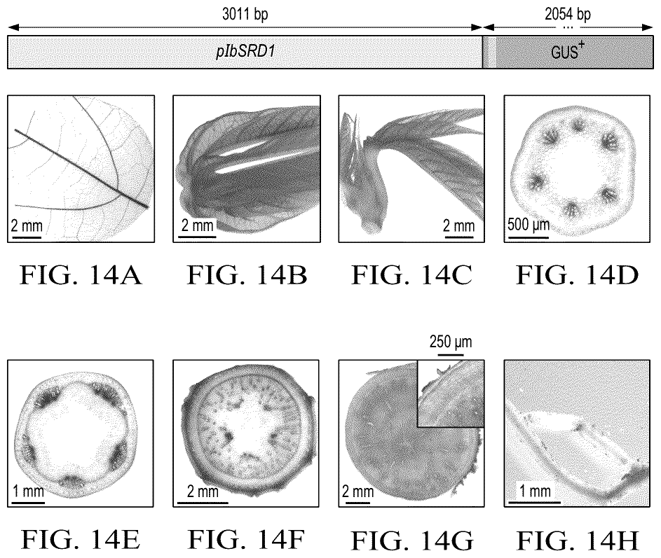

The promoter activity in cassava resembles the results obtained for sweet potato and thale cress. Pronounced staining was observed in the vasculature of source leaves, sink leaves, newly emerging leaves (FIG. 14A-C), and the vasculature of fibrous roots (FIG. 14H), as well as in the protoxylem and xylem vessels of petioles and stems (FIG. 14D-E). In addition, strong staining was observed in the vascular cambium and cork cambium of stems and storage roots (FIG. 14E-G). Together these results demonstrate that pIbSRD1 (SEQ ID NO: 16) has specific activity for cells with meristematic identity in cassava.

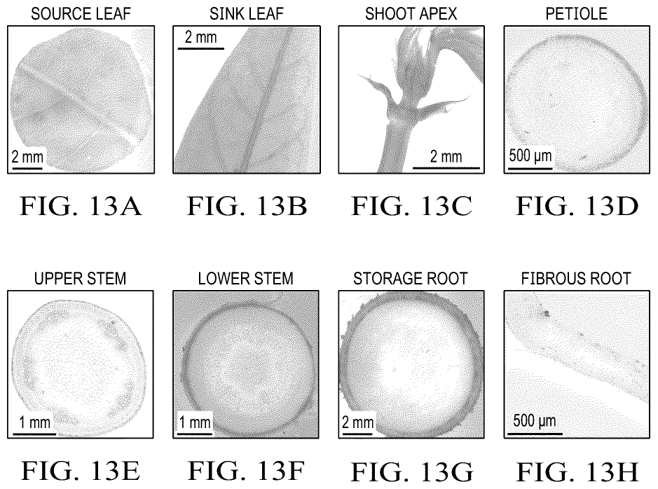

The promoter of Manes.14g071100 (SEQ ID NO: 4) was also tested based on expected ubiquitous activity due to its expected role in mitochondrial function. However, targeted testing using the particular promoter sequence of SEQ ID NO: 4 revealed the surprising but useful expression pattern shown in FIG. 13. Four lines were analyzed for this promoter::reporter combination. One line showed no staining but three lines showed a very similar pattern with different strength.

The pManes.14g071100::GUS lines displayed storage root cambium specificity along with minor activity in the shoot apex (FIG. 13).

The promoter of Manes.14g071100 (SEQ ID NO: 4) therefore has interesting applications for the expression of vascular cambium developmental regulators due to its specificity for storage root vascular cambium. This could potentially result in increased storage root size. This staining pattern supports the use of the promoter of pManes.14g071100 (SEQ ID NO: 4) as a new promoter element for developmental applications in cassava.

### Example 8: Summary of Promoter Element Specificities for Applications in Cassava

Among the tested leaf promoters, StFBPase_{cyt} (SEQ ID NO: 8), AtFBA2 (SEQ ID NO: 10), AtGAPA (SEQ ID NO: 12), StLS1 (SEQ ID NO: 15), and AtRBCS3B (SEQ ID NO: 20) displayed weak and/or non-specific expression. However, the promoters of AtCAB1 (SEQ ID NO: 6) and MePsbR (SEQ ID NO: 3) proved specific and relatively strong, making them well-suited tools for transgene expression in photosynthetic tissues of cassava. In particular, the results described herein, revealed the MePsbR promoter (SEQ ID NO: 3) as a viable means for expression of heterologous polynucleotide molecules in photosynthetic tissues in cassava. Although no dedicated promoter-GUS lines were created for the promoters of SlRBCS2 (SEQ ID NO: 19) and AtRBCS1A (SEQ ID NO: 18), they appeared very active in source leaf tissues in transcript studies. In addition, pSlRBCS2 (SEQ ID NO: 19) also appeared to be specific for this tissue.

The tested promoters of AtSUC2 (SEQ ID NO: 24), CmGolS1 (SEQ ID NO: 11), CoYMV (SEQ ID NO: 7), MeSWEET1-like (SEQ ID NO: 1), and StSTP1 (SEQ ID NO: 22) can be used as expression tools for phloem tissues, with several of these promoters having expression in specific phloem tissues. For example, while pAtSUC2 (SEQ ID NO: 24) has specific expression along the entire phloem, the promoters of pCmGolS1 (SEQ ID NO: 11) or pCoYMV (SEQ ID NO: 7) can target the loading or transport/unloading phloem, respectively. The promoter of MeSWEET1-like (SEQ ID NO: 1) and StSTP1 (SEQ ID NO: 22) can be used to target phloem and especially phloem parenchyma tissues of cassava. The promoter of MeSUS1 (SEQ ID NO: 5) also has considerable phloem activity, as well as storage tissue activity, especially in the cells closer to the vascular cambium. These results suggest that the MeSUS1 promoter sequence (SEQ ID NO: 5) can be an important tool for approaches centered on increased sink demand.

Among the promoters with predominant activity in heterotrophic storage tissue, MeGBSS1 (SEQ ID NO: 14) and StSSS3 (SEQ ID NO: 21) appeared to exhibit low specificity, or in the case of pStSSS3 (SEQ ID NO: 21) weak activity. The promoter of StPat (SEQ ID NO: 17) proved to be very active and very storage root-specific, as previously described. However, pStB33 (SEQ ID NO: 23), pMeGPT (SEQ ID NO: 2), and pStGBSS1 (SEQ ID NO: 13) are also very strong promoters for sink tissue expression, as they are predominantly active in starch-storing stem and storage root tissues. They also seem to have a comparable expression strength compared to StPat (SEQ ID NO: 17). These promoters will be useful to for use in larger transgene stacks that try to avoid repetition of the same promoter sequence in order to avoid silencing or recombination effects.

While pDjDIO3 (SEQ ID NO: 9) is likely very strong (as it showed a strong GUS staining within seconds of staining buffer addition), the promoter is less specific. In contrast, pIbSRD1 (SEQ ID NO: 16) showed a highly specific expression pattern with high activity in dividing cells. This promoter can be an important tool for more developmental focused approaches targeting stem cells.

Taken together, we have confirmed a number of tissue-specific promoter elements, allowing targeted transgene expression in a variety of cassava tissues. Furthermore, various promoter elements useful for expression of heterologous sequences in cassava have been described for the first time (e.g. MeSWEET1 (SEQ ID NO: 1), MeGPT (SEQ ID NO: 2), MePsbR (SEQ ID NO: 3), pManes.14g071100 (SEQ ID NO: 4), and MeSUS1 (SEQ ID NO: 5)).

A summary of the specific promoters per tissue is provided in Table 3. These promoter sequences will support further transgenic applications for spatial and temporal expression in cassava.

TABLE 3
Tissue specificity of promoters disclosed herein

| SEQ ID | | | Tissue | Results |
|---|---|---|---|---|
| NO: | Promoter | Code | Specificity | in FIG. |
| 1 | BIDIRECTIONAL | MeSWEET1 | Phloem and | 7 |
| | SUGAR | | phloem | |
| | TRANSPORTER | | parenchyma | |
| | SWEET1 | | | |
| 2 | GLUCOSE-6- | MeGPT | Heterotrophic | 12 |
| | PHOSPHATE/ | | tissues | |
| | PHOSPHATE | | | |
| | TRANSLOCATOR | | | |
| 3 | PHOTOSYSTEM II | MePsbR | Autotrophic | 3 |
| | SUBUNIT R | | tissues | |
| 4 | pManes.14g071100 | pManes.14g071100 | Storage root | 13 |
| 5 | SUCROSE | MeSUS1 | Phloem and | 8 |
| | SYNTHASE 1 | | parenchyma | |
| | | | cells | |
| 6 | CHLOROPHYLL A/B- | AtCAB1 | Autotrophic | 2 |
| | BINDING PROTEIN | | tissues | |
| 13 | GRANULE-BOUND | StGBSS1 | Heterotrophic | 11 |
| | STARCH SYNTHASE1 | | tissues | |
| 16 | MADS-BOX | IbSRD1 | Cambium and | 14 |
| | PROTEIN SRD1 | | metaxylem | |
| 17 | PATATIN CLASS 1 | StPat | Heterotrophic | 9 |
| | | | tissues | |
| 19 | RIBULOSE | SlRBCS2 | Autotrophic | 1 |
| | BISPHOSPHATE | | tissues | |
| | CARBOXYYLASE | | | |
| | SMALL SUBUNIT 2 | | | |
| 23 | B33 GENE | StB33 | Heterotrophic | 10 |
| | | | tissues | |
| 24 | SUCROSE-PROTON | AtSUC2 | Phloem | 4 |
| | SYMPORTER 2 | | companion cells | |

Having illustrated and described the principles of the present invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

What is claimed is:

1. A DNA molecule comprising a DNA sequence selected from the group consisting of:
a) a sequence with at least 85 percent sequence identity to any of SEQ ID NOs: 1-15, 17-19, and 21-24;
b) a sequence comprising any of SEQ ID NOs: 1-15, 17-19, and 21-24;
c) a fragment of a sequence having at least 85 percent sequence identity to any of SEQ ID NOs: 1-15, 17-19, and 21-24, wherein the fragment has gene-regulatory activity;
d) a fragment of any of SEQ ID NOs: 1-15, 17-19, and 21-24, wherein the fragment has gene-regulatory activity; and
e) combinations thereof,
wherein said sequence is operably linked to a heterologous transcribable polynucleotide molecule.

2. The DNA molecule of claim 1, wherein the DNA sequence is active as a promoter.

3. The DNA molecule of claim 1, wherein the DNA molecule further comprises a heterologous regulatory element.

4. The DNA molecule of claim 1, wherein said sequence has at least 90 percent sequence identity to the DNA sequence of any of SEQ ID NOs: 1-15, 17-19, and 21-24.

5. The DNA molecule of claim 1, wherein said sequence has at least 95 percent sequence identity to the DNA sequence of any of SEQ ID NOs: 1-15, 17-19, and 21-24.

6. The DNA molecule of claim 1, wherein the DNA sequence comprises gene regulatory activity.

7. The DNA molecule of claim 1, wherein the heterologous transcribable polynucleotide molecule comprises a gene of agronomic interest.

8. The DNA molecule of claim 7, wherein the gene of agronomic interest confers increased yield, increased root growth, or increased drought resistance in plants.

9. The DNA molecule of claim 7, wherein the gene of agronomic interest confers increased starch content in plants.

10. A construct comprising at least one copy of a DNA molecule of claim 1, and an operably linked transcribable gene of agronomic interest.

11. The construct of claim 10, wherein the construct comprises in the 5′-3′ direction: (a) the at least one copy of said DNA molecule; (b) the operably linked transcribable gene of agronomic interest; and (c) a gene termination sequence.

12. The construct of claim 10, wherein the transcribable gene of agronomic interest comprises an open reading frame encoding a polypeptide.

13. A transgenic plant cell comprising a heterologous DNA molecule comprising a sequence selected from the group consisting of:
a) a sequence with at least 85 percent sequence identity to any of SEQ ID NOs: 1-15, 17-19, and 21-24;
b) a sequence comprising any of SEQ ID NOs: 1-15, 17-19, and 21-24;
c) a fragment of a sequence having at least 85 percent sequence identity to any of SEQ ID NOs: 1-15, 17-19, and 21-24, wherein the fragment has gene-regulatory activity;
d) a fragment of any of SEQ ID NOs: 1-15, 17-19, and 21-24, wherein the fragment has gene-regulatory activity; and
e) combinations thereof,
wherein said sequence is operably linked to a heterologous transcribable polynucleotide molecule.

14. The transgenic plant cell of claim 13, wherein said transgenic plant cell is a monocotyledonous plant cell.

15. The transgenic plant cell of claim 13, wherein said transgenic plant cell is a dicotyledonous plant cell.

16. A transgenic plant, or part thereof, comprising the DNA molecule of claim 1.

17. A progeny plant of the transgenic plant of claim 16, or a part thereof, wherein the progeny plant or part thereof comprises said DNA molecule.

18. A transgenic seed, wherein the seed comprises the DNA molecule of claim 1.

19. A method of producing a commodity product comprising obtaining a transgenic plant or part thereof according to claim 16 and producing the commodity product therefrom.

20. The method of claim 19, wherein the commodity product is protein concentrate, protein isolate, grain, starch, seeds, meal, flour, biomass, or seed oil.

21. A commodity product comprising the DNA molecule of claim 1.

22. The commodity product of claim 21, wherein the commodity product is protein concentrate, protein isolate, grain, starch, seeds, meal, flour, biomass, or seed oil.

23. A method of expressing a transcribable polynucleotide molecule comprising obtaining a transgenic plant according to claim 16 and cultivating the plant, wherein the transcribable polynucleotide is expressed.

24. A method of expressing a gene of agronomic interest in a plant or plant cell, the method comprising incorporating into a plant cell a construct comprising the DNA molecule of claim 1, operably linked to a transcribable gene of agronomic interest, wherein the DNA molecule is capable of driving the expression of the operably linked gene of agronomic interest in the plant cell.

25. The method of claim 24, further comprising regenerating a transformed plant from said plant cell.

26. The method of claim 24, wherein said plant cell is stably transformed with said construct.

27. A method of producing a transgenic plant cell comprising introducing the DNA molecule of claim 1 into a plant cell.

28. The method of claim 27, wherein introducing said DNA molecule into said plant cell comprises transformation.

29. The method of claim 28, further comprising regenerating a transgenic plant from said plant cell.

30. The method of claim 27, wherein introducing said DNA molecule into said plant cell comprises crossing the transgenic plant of claim 16 with another plant to produce a progeny plant comprising said plant cell.
